SEQUENCE LISTING (252 sequences)

SEQ ID NO: 1 - DNA, 2109 bp, Artificial Sequence
  Synthesized pckA
aggaatgcgattccactcacaatattcccgccatataaaccaagatttaaccttttgagaacattttccacacctaaaatgctatttctgcgataatagcaaccgtttcgtgacaggaatcacggagttttttgtcaaatatgaatttctccagatacgtaaatctatgagccttgtcgcggttaacacccccaaaaagactttactattcaggcaatacatattggctaaggagcagtgaaatgcgcgttaacaatggtttgaccccgcaagaactcgaggcttatggtatcagtgacgtacatgatatcgtttacaacccaagctacgacctgctgtatcaggaagagctcgatccgagcctgacaggttatgagcgcggggtgttaactaatctgggtgccgttgccgtcgataccgggatcttcaccggtcgttcaccaaaagataagtatatcgtccgtgacgataccactcgcgatactttctggtgggcagacaaaggcaaaggtaagaacgacaacaaacctctctctccggaaacctggcagcatctgaaaggcctggtgaccaggcagctttccggcaaacgtctgttcgttgtcgacgctttctgtggtgcgaacccggatactcgtctttccgtccgtttcatcaccgaagtggcctggcaggcgcattttgtcaaaaacatgtttattcgcccgagcgatgaagaactggcaggtttcaaaccagactttatcgttatgaacggcgcgaagtgcactaacccgcagtggaaagaacagggtctcaactccgaaaacttcgtggcgtttaacctgaccgagcgcatgcagctgattggcggcacctggtacggcggcgaaatgaagaaagggatgttctcgatgatgaactacctgctgccgctgaaaggtatcgcttctatgcactgctccgccaacgttggtgagaaaggcgatgttgcggtgttcttcggcctttccggcaccggtaaaaccaccctttccaccgacccgaaacgtcgcctgattggcgatgacgaacacggctgggacgatgacggcgtgtttaacttcgaaggcggctgctacgcaaaaactatcaagctgtcgaaagaagcggaacctgaaatctacaacgctatccgtcgtgatgcgttgctggaaaacgtcaccgtgcgtgaagatggcactatcgactttgatgatggttcaaaaaccgagaacacccgcgtttcttatccgatctatcacatcgataacattgttaagccggtttccaaagcgggccacgcgactaaggttatcttcctgactgctgatgctttcggcgtgttgccgccggtttctcgcctgactgccgatcaaacccagtatcacttcctctctggcttcaccgccaaactggccggtactgagcgtggcatcaccgaaccgacgccaaccttctccgcttgcttcggcgcggcattcctgtcgctgcacccgactcagtacgcagaagtgctggtgaaacgtatgcaggcggcgggcgcgcaggcttatctggttaacactggctggaacggcactggcaaacgtatctcgattaaagatacccgcgccattatcgacgccatcctcaacggttcgctggataatgcagaaaccttcactctgccgatgtttaacctggcgatcccaaccgaactgccgggcgtagacacgaagattctcgatccgcgtaacacctacgcttctccggaacagtggcaggaaaaagccgaaaccctggcgaaactgtttatcgacaacttcgataaatacaccgacacccctgcgggtgccgcgctggtagcggctggtccgaaactgtaatgatttgaagctggagaatatctatccagtatcttatagaaagcaaaacgggaggcaccttcgcctcccgtttatttacccttcttttgtcgtgccctgcgcccgcgttaccggcactggcagccaggcgcgaatggaaagcccgccccgctcgctggtgccaagctccagcatcccgttatggttatccacgatacgctgcacaattgccagccctaatcccgtgccgctaatggtgcgcgca

SEQ ID NO: 2 - DNA, 2101 bp, Artificial Sequence
  Synthesized pckA
gacgctatcgataaattgaaaatgcagcaatagaggaaacacggtttgtttgagtgaaaacagccgtgttttttcatttaccgccataaaaatttgaaacggatcacaaatcatgaaaaaaatacgttcaaattagaactaattatcgaaaatttgatctagttaacattttttaggtataaatagttttaaaatagatctagtttggatttttaattttaaattatcaatgaggtgaagtatgactgacttaaacaaactcgttaaagaacttaatgacttagggcttaccgatgttaaggaaattgtgtataacccgagttatgaacaacttttcgaggaagaaaccaaaccgggtttggagggtttcgataaagggacgttaaccacgcttggcgcggttgccgtcgatacggggatttttaccggtcgttcaccgaaagataaatatatcgtttgcgatgaaactacgaaagacaccgtttggtggaacagcgaagcggcgaaaaacgataacaaaccgatgacgcaagaaacttggaaaagtttgagagaattagtggcgaaacaactttccggtaaacgtttattcgtggtagaaggttactgcggcgccagtgaaaaacaccgtatcggtgtgcgtatggttactgaagtggcatggcaggcgcattttgtgaaaaacatgtttatccgaccgaccgatgaagagttgaaaaatttcaaagcggattttaccgtgttaaacggtgctaaatgtactaatccgaactggaaagaacaaggtttgaacagtgaaaactttgtcgctttcaatattaccgaaggtattcagcttatcggcggtacttggtacggcggtgaaatgaaaaaaggtatgttctcaatgatgaactacttcctgccgttaaaaggtgtggcttccatgcactgttccgccaacgtaggtaaagacggtgacgtggctattttcttcggtttatccggtacgggtaaaacaacgctttcgaccgatcctaaacgccaattaatcggtgatgacgaacacggttgggatgaatccggcgtatttaactttgaaggcggttgttacgcgaaaaccattaacttatctcaagaaaacgaaccggatatttacggcgcaatccgtcgtgacgcattattagaaaacgtcgtggttcgtgcagacggttccgttgactttgacgacggttcaaaaacagaaaatacccgtgtttcatatccgatttaccacatcgacaacatcgttcgtccggtatcgaaagccggtcatgcaaccaaagtgattttcttaaccgcggacgcattcggcgtattgccgccggtttcaaaactgactccggaacaaaccgaatactacttcttatccggctttactgcaaaattagcgggtacggaacgcggcgtaaccgaaccgactccgacattctcggcctgtttcggtgcggcattcttaagcctgcatccgattcaatatgcggacgtgttggtcgaacgcatgaaagcctccggtgcggaagcttatttggtgaacaccggttggaacggcacgggtaaacgtatttcaatcaaagatacccgcggtattatcgatgcgattttggacggttcaatcgaaaaagcggaaatgggcgaattgccaatctttaatttagcgattcctaaagcattaccgggtgttgatcctgctattttggatccgcgcgatacttacgcagacaaagcgcaatggcaagttaaagcggaagatttggcaaaccgtttcgtgaaaaactttgtgaaatatacggcgaatccggaagcggctaaattagttggcgccggtccaaaagcataaaactgtaaaagcatagtatgtgcatgattcggtaaactaccgaataaaatctgaaaaataaaggctgagtatttccactcagccttttgttttggaaattagaagttgtttagaaagataaacggcgcggcttattcgtccgtattatttggggacataaaaaacccgttgataaacaacgggtttgtcatcttaacaggttattaggccgccggcacaaatcctactgcgtcataaaccgcc

SEQ ID NO: 3 - DNA, 1221 bp, Artificial Sequence
  Synthesized mdh1
cggcaattaaatgcataaacgctaaacttgcgtgactacacattcttgagatgtggtcattgtaaacggcaattttgtggattaaggtcgcggcagcggagcaacatatcttagtttatcaatataataaggagtttaggatgaaagtcgcagtcctcggcgctgctggcggtattggccaggcgcttgcactactgttaaaaacccaactgccttcaggttcagaactctctctgtatgatatcgctccagtgactcccggtgtggctgtcgatctgagccatatccctactgctgtgaaaatcaaaggtttttctggtgaagatgcgactccggcgctggaaggcgcagatgtcgttcttatctctgcaggcgtagcgcgtaaaccgggtatggatcgttccgacctgtttaacgttaacgccggcatcgtgaaaaacctggtacagcaagttgcgaaaacctgcccgaaagcgtgcattggtattatcactaacccggttaacaccacagttgcaattgctgctgaagtgctgaaaaaagccggtgtttatgacaaaaacaaactgttcggcgttaccacgctggatatcattcgttccaacacctttgttgcggaactgaaaggcaaacagccaggcgaagttgaagtgccggttattggcggtcactctggtgttaccattctgccgctgctgtcacaggttcctggcgttagttttaccgagcaggaagtggctgatctgaccaaacgcatccagaacgcgggtactgaagtggttgaagcgaaggccggtggcgggtctgcaaccctgtctatgggccaggcagctgcacgttttggtctgtctctggttcgtgcactgcagggcgaacaaggcgttgtcgaatgtgcctacgttgaaggcgacggtcagtacgcccgtttcttctctcaaccgctgctgctgggtaaaaacggcgtggaagagcgtaaatctatcggtaccctgagcgcatttgaacagaacgcgctggaaggtatgctggatacgctgaagaaagatatcgccctgggcgaagagttcgttaataagtaattgattagcggataataaaaaaccggagcacagactccggttttttgttttgagcgcacgacttaattggttgccggatattcctgaatggtgacctgcagcgttaactgcttatcatcacgcatcactactacagggatca

SEQ ID NO: 4 - DNA, 1474 bp, Artificial Sequence
  Synthesized mdh2
agatttatcgatatgagataaagattgctgcatgattctccttctgattctttttccctgtatatattttctccccttctgtataaatcgtacagtcagaagtagtccagaatatagtgctgcagactattacaaaagttcaatacaatatcataaaagttatagtaacatgcctcactcagttacaccatccatagaacaagattcgttaaaaattgccattttaggtgctgccggtggtatcgggcagtcgttatcgctgcttttgaaagctcagttgcaataccagttaaaggagagcaaccggagcgttacccacattcatctggctctttacgatgtcaaccaagaagccatcaacggtgttaccgccgacttgtctcatatagacacccccatttccgtgtcgagccactctcctgcaggtggcattgagaactgtttgcataacgcttctattgttgtcattcctgcaggtgttccaagaaaacctggcatgactcgtgatgacttatttaacgtgaatgctggtatcattagccagctcggtgattctattgcagaatgttgtgatctttccaaggtcttcgttcttgtcatttccaaccctgttaattctttagtcccagtgatggtttctaacattcttaagaaccatcctcagtctagaaattccggcattgaaagaaggatcatgggtgtcaccaagctcgacattgtcagagcgtccacttttctacgtgagataaacattgagtcagggctaactcctcgtgttaactccatgcctgacgtccctgtaattggcgggcattctggcgagactattattccgttgttttcacagtcaaacttcctatcgagattaaatgaggatcaattgaaatatttaatacatagagtccaatacggtggtgatgaagtggtcaaggccaagaacggtaaaggtagtgctaccttatcgatggcccatgccggttataagtgtgttgtccaatttgtttctttgttattgggtaacattgagcagatccatggaacctactatgtgccattaaaagatgcgaacaacttccccattgctcctggggcagatcaattattgcctctggtggacggtgcagactactttgccataccattaactattactacaaagggtgtttcctatgtggattatgacatcgttaataggatgaacgacatggaacgcaaccaaatgttgccaatttgcgtctcccagttaaagaaaaatatcgataagggcttggaattcgttgcatcgagatctgcatcatcttaatgagcatcggaccgaagcataagaatgcagcaaattgataatattcccgttaagccagtcgtaggaaaaggaaaagaatgagtagcaaatgtcacacaacaaacgcgcaccatccctacatacacaaacacacacacacacacacatatatatatattctaccccatataa

SEQ ID NO: 5 - DNA, 2141 bp, Artificial Sequence
  Synthesized mqo
cgcgtgatgccgttgcccggacggcataatattacgacgctgacctggcggactgctgccgtcaggtcaatatgctaaaaatcccttcatttctaataaccctataattaatcaacgaaattataatgtttctaaaattagaatataatttataaacattatttaaatgttgttacttaagtgttaccgttgatgccgcgcaaatctcacctgcaataaagcgactaaaagtaaggcattaacaagatgaaaaaagtgactgccatgctcttctcgatggccgtggggcttaatgccgtttcgatggcggcaaaagcgaaagcgtccgaggagcaggaaactgatgtactgttgattggcggcggcattatgagcgccacgttggggacctatttacgcgagctggagcctgaatggtcgatgaccatggtggagcgcctggagggtgtcgcgcaggagagttcgaacggctggaataacgccggaaccgggcattctgcactgatggaactgaactacaccccgcaaaacgccgatggcagcatcagtattgaaaaagcagtcgccattaacgaagcatttcagatttcccgccagttctgggcgcaccaggttgagcgcggcgtgctgcgtactccgcgttcatttatcaataccgttccgcatatgagctttgtctggggcgaggataacgtcaatttcctgcgcgcccgttacgccgcgttgcaacaaagctcgctgtttcgcggtatgcgttactctgaagatcacgcgcagatcaaagagtgggcaccgttagtgatggaagggcgcgatccgcaacagaaagtggcagccacgcgtacggaaattggtaccgatgtgaactacggcgagatcacccgccagttaattgcttccttgcagaagaaatctaacttctcgctgcaactcagcagcgaagtccgcgccctaaagcgtaatgacgataacacctggaccgttaccgttgccgatctgaaaaatggcactgcacagaacattcgtgccaaatttgtctttatcggcgcgggcggtgcggcgctgaagctgttacaggaatcggggattccggaagcgaaagactacgccggtttcccggtgggcggacagttccttgtttcggaaaacccggacgtggttaatcaccatctggcgaaggtttacggtaaagcatccgttggcgcaccaccgatgtcggttccgcatatcgatacccgcgttctggacggtaaacgcgtagtgctgtttgggccatttgccaccttctcaaccaaattcctcaaaaacggttcattgtgggatctaatgagttccaccaccacctctaacgtgatgccgatgatgcacgtcgggctggataatttcgatctggtgaaatatctggtgagtcaggtgatgttgagtgaagaggatcgttttgaagcgttgaaagagtactatccgcaggcgaaaaaagaggactggcgtttgtggcaagcggggcagcgcgtgcagattatcaagcgtgatgccgagaaaggtggcgtactgcgtctgggtactgaagtcgtcagtgaccagcaaggaaccattgccgcgctcctgggggcatcgccaggggcgtcaaccgccgcgccgattatgttgaatctgctggaaaaagtatttggcgatcgtgtttccagcccgcaatggcaggctacgttgaaagcgatcgttccgtcttatggacgcaagctgaacggtgatgtagcggcaacagaacgcgagttgcagtacaccagcgaagtgctggggttgaactacgacaagccgcaggcagcagatagtacgccgaaaccgcagttgaaaccgcaacccgttcaaaaagaagtggcggatattgcgttgtaatgatacgccacatccggcatggtatgccggatgtggcgtatgctgataagacgcgccagcgtcgcatcaggcaaccggctcgggcgttagatgttggatgcggcgtaaccgccttatccaacctacggggtggagcacttcacagcggtgtaggcctgataagacgcgccagcgtcgcatcaggcaaccggcttgggcgttagatgtcggatgcggcgtaaccgccttatccgacctacggggtggag

SEQ ID NO: 6 - DNA, 1907 bp, Artificial Sequence
  Synthesized fum1
cactgttgtagacgttaaacaagcatccaacaggaggccggaccgaacacacgggggagtgactctagctctttcttctgggatattcatatcgtttctccattacatacagtcatttttttgcatttttaaattcgtgacttttagtactgcagctgttttttttctcacgtcaagaaattccataaagtctaactattaaacggataagagatacaatcatgttgagatttaccaattgtagttgcaagactttcgtaaaatcgtcatataagcttaatataagaagaatgaactcctcgttcagaactgaaaccgatgcatttggtgagatacacgtgcctgctgataagtactggggtgcccaaactcaaagatcctttcaaaacttcaagattggtggcgctcgtgaaagaatgccattgcctttggtgcatgcatttggtgttttgaagaaatctgccgcgattgtgaatgagtctctggggggattggatcccaagatctccaaggctattcaacaggccgctgacgaagtggcttctggtaagctagacgaccactttccattggttgttttccaaacgggttccggtacccagtctaacatgaatgctaatgaagttatttccaatcgtgccattgagatcctaggaggcaagattggatctaaacaagtccatccaaacaatcattgcaaccaatctcaatcatccaatgatactttccccactgtcatgcatatcgctgccagtttgcaaattcaaaacgagttgatacctgagttgaccaatttaaagaacgcccttgaagccaaatccaaggaatttgaccatattgtgaagatcggtagaacacacttgcaagatgccacgcctttgacactaggtcaagagttttccggttacgtgcaacaagttgagaacggtatccaaagagtggcacattctttgaaaacattgagtttcctggcacaaggtggtactgccgttggtacagggttgaacaccaagcccggattcgatgtcaagatagccgagcaaatttccaaagaaactggactaaagtttcaaaccgcccctaataagttcgaggctttggctgctcacgacgccattgtcgaatgtagcggtgctctaaacacccttgcttgttctcttttcaaaatagcgcaagatataagatacttagggtccgggccacgttgcggctaccatgaactaatgctgccagagaatgaaccaggttcctcaatcatgcctggtaaagttaacccaacccaaaacgaggcattgactcaagtgtgtgtgcaagtcatgggtaacaatgcagctatcacgtttgccggctctcaaggtcaattcgagttaaatgtcttcaagccagtcatgatcgccaatctattgaattcgatcaggttaattactgatgccgcatattcatttagagtgcactgtgttgaaggtatcaaagccaatgagcctcgtattcatgagttgttgactaaatctttaatgttagtcaccgctttgaaccctaagatcggttatgatgccgcttccaaggtcgccaagaatgctcataagaagggcattaccttgaaggaaagtgcattggaattgggtgtattgactgaaaaggaatttgatgaatgggttgttcctgaacacatgctaggtcctaaataaacgagctaaatacctaataatatacaagttttttatgtcttattatatgaaggaaaataaggaagtggagagaatttgtgattcagcaatggtccctccgctaaggtcccgcctctggccagttatgtcagaagaggagcataggcatacattccatttttgatggcttaatggggcccataaatttacatcactacattattattcctatacaagtta

SEQ ID NO: 7 - DNA, 2141 bp, Artificial Sequence
  Synthesized fumA
tttgctgccccaggcatagttttgcactgagttaatgagtttttgcatgatcaatccctgttttaatgtggaaattaatcccactattaaagcaagaatcctacgggaagtaacctggagccgcaaaaagtcgtactagtctcagtttttgttaaaaaagtgtgtaggatattgttactcgcttttaacagggcaacggaacacccgcccagagcataaccaaaccaggcagtaagtgagagaacaatgtcaaacaaaccctttcattatcaggctccttttccactcaaaaaagatgatactgagtattacctgctaaccagcgaacacgttagcgtatctgaatttgaagggcaggagattttgaaagtcgcacccgaagcgttaactctgttggcgcgccaggcgtttcatgatgcgtcgttcatgctgcgtccggcgcaccaacaacaggtggccgacattctgcgtgacccggaggccagcgaaaatgataaatatgtggcgctgcaattcctgcgtaactccgacatcgcggcgaaaggcgttctgccaacctgtcaggataccggcaccgcgattattgttggtaaaaaagggcagcgtgtatggaccggtggtggtgatgaagcggcgctggcgcgcggtgtctataacacttatatcgaagataatctgcgctactcgcaaaacgcgccgctggatatgtataaagaagtgaataccggcaccaatctgccagcgcagatcgatctttatgccgttgatggcgacgaatacaaattcctctgtatcgccaaaggtggtggttcggcaaacaagacgtatctctatcaggaaaccaaagcgttactgacgccggggaaactgaaaaattacctggttgagaagatgcgcacgctgggtacggcggcctgtcctccgtatcatattgcgttcgttattggtggaacttctgcagaaacgaaccttaaaacggtgaaactggcttccgcgaaatactatgatgaactgccaacggaagggaatgagcacggtcaggcgttccgcgatgtggaactggaaaaagaattgctgatcgaagcgcaaaatcttggtctgggtgcgcagtttggtggtaaatacttcgctcacgacatccgcgtgattcgcctgccacgtcacggcgcatcctgcccggtcggtatgggcgtctcctgctctgctgaccgtaatatcaaagcgaagatcaaccgtcaggggatctggatcgaaaaactggaacataatccaggcaaatatatcccggaagagctgcgcaaagcgggagaaggcgaagcggtgcgcgttgaccttaaccgtccgatgaaagagatcctcgcacagttgtcgcagtatcccgtttctacacgcttatcgcttaacggcacgattatcgtcggtcgtgatattgctcacgccaaactgaaagagcggatggataacggtgaagggctgccgcagtacatcaaagatcatccgatttactacgcgggtccggccaaaacgccggaaggttatgcctccggttctcttggcccaacgaccgccggacggatggattcttatgtcgatcaactgcaagcgcagggcggaagtatgatcatgctggcgaaaggcaaccgcagccagcaggtgacggatgcctgtaaaaaacacggcggcttctaccttggcagtatcggtggtccggccgctgtattggcgcagggaagtattaagagcctggaatgtgttgaatatccggaactgggaatggaagccatctggaaaattgaagtggaagatttcccggcgtttatccttgtggatgataaaggaaatgacttcttccagcagatacaactcacacaatgcacccgctgtgtgaaataaacagagccgcccttcggggcggtttttttacatggcacgaaagaccaaacatttgttatcaaatggtaaataataagtgagctaaaagttgcttaacgaaagcaaaacagaaagaaaaaattaatcaggtgaggagcaggtcatgaatacagtacgcagcgaaaaagattcgatgggggcgattgatgtcccggcagataagctgtggggcgcacaaactcaacgctcgctggagcatttccgcattt

SEQ ID NO: 8 - DNA, 2141 bp, Artificial Sequence
  Synthesized fumB
cctgccgacttatccgagcgatctggcagcgattcagtttgaccgttccggcaccacccacatcggtcgcttcgtcatcaaccacagctttattctgccggggttgattggtgtgagcgtatcgtgcgtcttcggctggatcttcgccgcgatgtacgggttcttataaatgcactttgcgtgccgcccgtgactacgcggcacgccattttcgaataacaaatacagagttacaggctggaagctatgtcaaacaaaccctttatctaccaggcacctttcccgatggggaaagacaataccgaatactatctactcacttccgattacgttagcgttgccgacttcgacggcgaaaccatcctgaaagtggaaccagaagccctgaccctgctggcgcagcaagcctttcacgacgcttcttttatgctccgcccggcacaccagaaacaggttgcggctattcttcacgatccagaagccagcgaaaacgacaagtacgtggcgctgcaattcttaagaaactccgaaatcgccgccaaaggcgtgctgccgacctgccaggataccggcaccgcgatcatcgtcggtaaaaaaggccagcgcgtgtggaccggcggcggtgatgaagaaacgctgtcgaaaggcgtctataacacctatatcgaagataacctgcgctattcacagaatgcggcgctggacatgtacaaagaggtcaacaccggcactaacctgcctgcgcaaatcgacctgtacgcggtagatggcgatgagtacaaattcctttgcgttgcgaaaggcggcggctctgccaacaaaacgtatctctaccaggaaaccaaagccctgctgactcccggcaaactgaaaaacttcctcgtcgagaaaatgcgtaccctcggtactgcagcctgcccgccgtaccatatcgcgtttgtgattggcggtacgtctgcggaaaccaacctgaaaaccgtcaagttagcaagcgctcactattacgatgaactgccgacggaagggaacgaacatggtcaggcgttccgcgatgtccagctggaacaggaactgctggaagaggcccagaaactcggtcttggcgcgcagtttggcggtaaatacttcgcgcacgacattcgcgttatccgtctgccacgtcacggcgcatcctgcccggtcggcatgggcgtctcctgctccgctgaccgtaacattaaagcgaaaatcaaccgcgaaggtatctggatcgaaaaactggaacacaacccaggccagtacattccacaagaactgcgccaggccggtgaaggcgaagcggtgaaagttgaccttaaccgcccgatgaaagagatcctcgcccagctttcgcaatacccggtatccactcgtttgtcgctcaccggcaccattatcgtgggccgagatattgcacacgccaagctgaaagagctgattgacgccggtaaagaacttccgcagtacatcaaagatcacccgatctactacgcgggtccggcgaaaacccctgccggttatccatcaggttcacttggcccaaccaccgcaggccgtatggactcctacgtggatctgctgcaatcccacggcggcagcatgatcatgctggcgaaaggtaaccgcagtcagcaggttaccgacgcgtgtcataaacacggcggcttctacctcggtagcatcggcggtccggcggcggtactggcgcagcagagcatcaagcatctggagtgcgtcgcttatccggagctgggtatggaagctatctggaaaatcgaagtagaagatttcccggcgtttatcctggtcgatgacaaaggtaacgacttcttccagcaaatcgtcaacaaacagtgcgcgaactgcactaagtaacctcttcggcccagcgcctggcagcatgctgccaggtgatccccctggccacctcttttgcgattgtaatttcacgcttgctggtgaatagtcagtattttccctgatttgcgaactcaccatgacccgcacactcaagccgttaattcttaacaccagcgcactgacgctaacgttaatcctgatttataccggcatttcggcccatgacaaactcacctggctgatggaagtgacaccggtgatta

SEQ ID NO: 9 - DNA, 1824 bp, Artificial Sequence
  Synthesized fumC
ggatgataaaggaaatgacttcttccagcagatacaactcacacaatgcacccgctgtgtgaaataaacagagccgcccttcggggcggtttttttacatggcacgaaagaccaaacatttgttatcaaatggtaaataataagtgagctaaaagttgcttaacgaaagcaaaacagaaagaaaaaattaatcaggtgaggagcaggtcatgaatacagtacgcagcgaaaaagattcgatgggggcgattgatgtcccggcagataagctgtggggcgcacaaactcaacgctcgctggagcatttccgcatttcgacggagaaaatgcccacctcactgattcatgcgctggcgctaaccaagcgtgcagcggcaaaagttaatgaagatttaggcttgttgtctgaagagaaagcgagcgccattcgtcaggcggcggatgaagtactggcaggacagcatgacgacgaattcccgctggctatctggcagaccggctccggcacgcaaagtaacatgaacatgaacgaagtgctggctaaccgggccagtgaattactcggcggtgtgcgcgggatggaacgtaaagttcaccctaacgacgacgtgaacaaaagccaaagttccaacgatgtctttccgacggcgatgcacgttgcggcgctgctggcgctgcgcaagcaactcattcctcagcttaaaaccctgacacagacactgaatgagaaatcccgtgcttttgccgatatcgtcaaaattggtcgtactcacttgcaggatgccacgccgttaacgctggggcaggagatttccggctgggtagcgatgctcgagcataatctcaaacatatcgaatacagcctgcctcacgtagcggaactggctcttggcggtacagcggtgggtactggactaaatacccatccggagtatgcgcgtcgcgtagcagatgaactggcagtcattacctgtgcaccgtttgttaccgcgccgaacaaatttgaagcgctggcgacctgtgatgccctggttcaggcgcacggcgcgttgaaagggttggctgcgtcactgatgaaaatcgccaatgatgtccgctggctggcctctggcccgcgctgcggaattggtgaaatctcaatcccggaaaatgagccgggcagctcaatcatgccggggaaagtgaacccaacacagtgtgaggcattaaccatgctctgctgtcaggtgatggggaacgacgtggcgatcaacatggggggcgcttccggtaactttgaactgaacgtcttccgtccaatggtgatccacaatttcctgcaatcggtgcgcttgctggcagatggcatggaaagttttaacaaacactgcgcagtgggtattgaaccgaatcgtgagcgaatcaatcaattactcaatgaatcgctgatgctggtgactgcgcttaacacccacattggttatgacaaagccgccgagatcgccaaaaaagcgcataaagaagggctgaccttaaaagctgcggcccttgcgctggggtatcttagcgaagccgagtttgacagctgggtacggccagaacagatggtcggcagtatgaaagccgggcgttaatctgcaacatacaggtgcagccgtggaatgatcaaacgaagcggctgcgcctgaggtttataacggtgctgcacattgtcggcatcgtaatttaacaactcaccaacgtccggcacgcccgcgccattatcccgattaatcagtgcaatcagtggtgtagggcaggcgtgttggacttgtttttgatctcctttgtaccagacgcgggcaa

SEQ ID NO: 10 - DNA, 2351 bp, Artificial Sequence
  Synthesized frdA
gagacaaattttacgcaggaatcaaacagcggtgggcagtgactaaaaaaagcacgatctgatggtttagtaattaaattaatcatcttcagtgataatttagccctcttgcgcactaaaaaaatcgatctcgtcaaatttcagacttatccatcagactatactgttgtacctataaaggagcagtggaatagcgttcgcagaccgtaactttcaggtacttaccctgaagtacgtggctgtgggataaaaacaatctggaggaatgtcgtgcaaacctttcaagccgatcttgccattgtaggcgccggtggcgcgggattacgtgctgcaattgctgccgcgcaggcaaatccgaatgcaaaaatcgcactaatctcaaaagtatacccgatgcgtagccataccgttgctgcagaagggggctccgccgctgtcgcgcaggatcatgacagcttcgaatatcactttcacgatacagtagcgggtggcgactggttgtgtgagcaggatgtcgtggattatttcgtccaccactgcccaaccgaaatgacccaactggaactgtggggatgcccatggagccgtcgcccggatggtagcgtcaacgtacgtcgcttcggcggcatgaaaatcgagcgcacctggttcgccgccgataagaccggcttccatatgctgcacacgctgttccagacctctctgcaattcccgcagatccagcgttttgacgaacatttcgtgctggatattctggttgatgatggtcatgttcgcggcctggtagcaatgaacatgatggaaggcacgctggtgcagatccgtgctaacgcggtcgttatggctactggcggtgcgggtcgcgtttatcgttacaacaccaacggcggcatcgttaccggtgacggtatgggtatggcgctaagccacggcgttccgctgcgtgacatggaattcgttcagtatcacccaaccggtctgccaggttccggtatcctgatgaccgaaggttgccgcggtgaaggcggtattctggtcaacaaaaatggctaccgttatctgcaagattacggcatgggcccggaaactccgctgggcgagccgaaaaacaaatatatggaactgggtccacgcgacaaagtctctcaggccttctggcacgaatggcgtaaaggcaacaccatctccacgccgcgtggcgatgtggtttatctcgacttgcgtcacctcggcgagaaaaaactgcatgaacgtctgccgttcatctgcgaactggcgaaagcgtacgttggcgtcgatccggttaaagaaccgattccggtacgtccgaccgcacactacaccatgggcggtatcgaaaccgatcagaactgtgaaacccgcattaaaggtctgttcgccgtgggtgaatgttcctctgttggtctgcacggtgcaaaccgtctgggttctaactccctggcggaactggtggtcttcggccgtctggccggtgaacaagcgacagagcgtgcagcaactgccggtaatggcaacgaagcggcaattgaagcgcaggcagctggcgttgaacaacgtctgaaagatctggttaaccaggatggcggcgaaaactgggcgaagatccgcgacgaaatgggcctggctatggaagaaggctgcggtatctaccgtacgccggaactgatgcagaaaaccatcgacaagctggcagagctgcaggaacgcttcaagcgcgtgcgcatcaccgacacttccagcgtgttcaacaccgacctgctctacaccattgaactgggccacggtctgaacgttgctgaatgtatggcgcactccgcaatggcacgtaaagagtcccgcggcgcgcaccagcgtctggacgaaggttgcaccgagcgtgacgacgtcaacttcctcaaacacaccctcgccttccgcgatgctgatggcacgactcgcctggagtacagcgacgtgaagattactacgctgccgccagctaaacgcgtttacggtggcgaagcggatgcagccgataaggcggaagcagccaataagaaggagaaggcgaatggctgagatgaaaaacctgaaaattgaggtggtgcgctataacccggaagtcgataccgcaccgcatagcgcattctatgaagtgccttatgacgcaactacctcattactggatgcgctgggctacatcaaagacaacctggcaccggacctgagctaccgctggtcctgccgtatggcgatttgtggttcctgcggcatgatggttaacaacgtgccaaaactggcatgtaaaaccttcctgcgtgattacaccgacggtatgaaggttgaagcgt

SEQ ID NO: 11 - DNA, 955 bp, Artificial Sequence
  Synthesized frdB
gagtacagcgacgtgaagattactacgctgccgccagctaaacgcgtttacggtggcgaagcggatgcagccgataaggcggaagcagccaataagaaggagaaggcgaatggctgagatgaaaaacctgaaaattgaggtggtgcgctataacccggaagtcgataccgcaccgcatagcgcattctatgaagtgccttatgacgcaactacctcattactggatgcgctgggctacatcaaagacaacctggcaccggacctgagctaccgctggtcctgccgtatggcgatttgtggttcctgcggcatgatggttaacaacgtgccaaaactggcatgtaaaaccttcctgcgtgattacaccgacggtatgaaggttgaagcgttagctaacttcccgattgaacgcgatctggtggtcgatatgacccacttcatcgaaagtctggaagcgatcaaaccgtacatcatcggcaactcccgcaccgcggatcagggtactaacatccagaccccggcgcagatggcgaagtatcaccagttctccggttgcatcaactgtggtttgtgctacgccgcgtgcccgcagtttggcctgaacccagagttcatcggtccggctgccattacgctggcgcatcgttataacgaagatagccgcgaccacggtaagaaggagcgtatggcgcagttgaacagccagaacggcgtatggagctgtactttcgtgggctactgctccgaagtctgcccgaaacacgtcgatccggctgcggccattcagcagggcaaagtagaaagttcgaaagactttcttatcgcgaccctgaaaccacgctaaggagtgcaacatgacgactaaacgtaaaccgtatgtacggccaatgacgtccacctggtggaaaaaattgccgttttatcgcttttacatgctgcgcgaaggcacggcggt

SEQ ID NO: 12 - DNA, 514 bp, Artificial Sequence
  Synthesized frdC
gtagaaagttcgaaagactttcttatcgcgaccctgaaaccacgctaaggagtgcaacatgacgactaaacgtaaaccgtatgtacggccaatgacgtccacctggtggaaaaaattgccgttttatcgcttttacatgctgcgcgaaggcacggcggttccggctgtgtggttcagcattgaactgattttcgggctgtttgccctgaaaaatggcccggaagcctgggcgggattcgtcgactttttacaaaacccggttatcgtgatcattaacctgatcactctggcggcagctctgctgcacaccaaaacctggtttgaactggcaccgaaagcggccaatatcattgtaaaagacgaaaaaatgggaccagagccaattatcaaaagtctctgggcggtaactgtggttgccaccatcgtaatcctgtttgttgccctgtactggtaaggagcctgagatgattaatccaaatccaaagcgttctgacgaaccggtattctggggcct

SEQ ID NO: 13 - DNA, 468 bp, Artificial Sequence
  Synthesized frdD
ggttgccaccatcgtaatcctgtttgttgccctgtactggtaaggagcctgagatgattaatccaaatccaaagcgttctgacgaaccggtattctggggcctcttcggggccggtggtatgtggagcgccatcattgcgccggtgatgatcctgctggtgggtattctgctgccactggggttgtttccgggtgatgcgctgagctacgagcgcgttctggcgttcgcgcagagcttcattggtcgcgtattcctgttcctgatgatcgttctgccgctgtggtgtggtttacaccgtatgcaccacgcgatgcacgatctgaaaatccacgtacctgcgggcaaatgggttttctacggtctggctgctatcctgacagttgtcacgctgattggtgtcgttacaatctaacgcatcgccaatgtaaatccggcccgcctatggcgggccgttttgtatggaaacc

SEQ ID NO: 14 - DNA, 2297 bp, Artificial Sequence
  Synthesized sdhA
ctctacatcatttatatggtcggttttttcgctaccagtggcgagctgacatatgaagtctggatcggtttcttcgcctctgcgttcaccaaagtgttcaccctgctggcgctgttttctatcttgatccatgcctggatcggcatgtggcaggtgttgaccgactacgttaaaccgctggctttgcgcctgatgctgcaactggtgattgtcgttgcactggtggtttacgtgatttatggattcgttgtggtgtggggtgtgtgatgaaattgccagtcagagaatttgatgcagttgtgattggtgccggtggcgcaggtatgcgcgcggcgctgcaaatttcccagagcggccagacctgtgcgctgctctctaaagtcttcccgacccgttcccataccgtttctgcgcaaggcggcattaccgttgcgctgggtaatacccatgaagataactgggaatggcatatgtacgacaccgtgaaagggtcggactatatcggtgaccaggacgcgattgaatatatgtgtaaaaccgggccggaagcgattctggaactcgaacacatgggcctgccgttctcgcgtctcgatgatggtcgtatctatcaacgtccgtttggcggtcagtcgaaaaacttcggcggcgagcaggcggcacgcactgcggcagcagctgaccgtaccggtcacgcactgttgcacacgctttatcagcagaacctgaaaaaccacaccaccattttctccgagtggtatgcgctggatctggtgaaaaaccaggatggcgcggtggtgggttgtaccgcactgtgcatcgaaaccggtgaagtggtttatttcaaagcccgcgctaccgtgctggcgactggcggagcagggcgtatttatcagtccaccaccaacgcccacattaacaccggcgacggtgtcggcatggctatccgtgccggcgtaccggtgcaggatatggaaatgtggcagttccacccgaccggcattgccggtgcgggcgtactggtcaccgaaggttgccgtggtgaaggcggttatctgctgaacaaacatggcgaacgttttatggagcgttatgcgccgaacgccaaagacctggcgggccgtgacgtggttgcgcgttccatcatgatcgaaatccgtgaaggtcgcggctgtgatggtccgtgggggccacacgcgaaactgaaactcgatcacctgggtaaagaagttctcgaatcccgtctgccgggtatcctggagctttcccgtaccttcgctcacgtcgatccggtgaaagagccgattccggttatcccaacctgtcactacatgatgggcggtattccgaccaaagttaccggtcaggcactgactgtgaatgagaaaggcgaagatgtggttgttccgggactgtttgccgttggtgaaatcgcttgtgtatcggtacacggcgctaaccgtctgggcggcaactcgctgctggacctggtggtctttggtcgcgcggcaggtctgcatctgcaagagtctatcgccgagcagggcgcactgcgcgatgccagcgagtctgatgttgaagcgtctctggatcgcctgaaccgctggaacaataatcgtaacggtgaagatccggtggcgatccgtaaagcgctgcaagaatgtatgcagcataacttctcggtcttccgtgaaggtgatgcgatggcgaaagggcttgagcagttgaaagtgatccgcgagcgtctgaaaaatgcccgtctggatgacacttccagcgagttcaacacccagcgcgttgagtgcctggaactggataacctgatggaaacggcgtatgcaacggctgtttctgccaacttccgtaccgaaagccgtggcgcgcatagccgcttcgacttcccggatcgtgatgatgaaaactggctgtgccactccctgtatctgccagagtcggaatccatgacgcgccgaagcgtcaacatggaaccgaaactgcgcccggcattcccgccgaagattcgtacttactaatgcggagacaggaaaatgagactcgagttttcaatttatcgctataacccggatgttgatgatgctccgcgtatgcaggattacaccctggaagcggatgaaggtcgcgacatgatgctgctggatgcgcttatccagctaaaagagaaagatcccagcctgtcgttccgccgctcctgccgtgaaggtgtgtgcggttccgacggtctgaacatgaacggcaagaatggtctggcctgtattaccccgatttcggcactcaac

SEQ ID NO: 15 - DNA, 931 bp, Artificial Sequence
  Synthesized sdhB
tatctgccagagtcggaatccatgacgcgccgaagcgtcaacatggaaccgaaactgcgcccggcattcccgccgaagattcgtacttactaatgcggagacaggaaaatgagactcgagttttcaatttatcgctataacccggatgttgatgatgctccgcgtatgcaggattacaccctggaagcggatgaaggtcgcgacatgatgctgctggatgcgcttatccagctaaaagagaaagatcccagcctgtcgttccgccgctcctgccgtgaaggtgtgtgcggttccgacggtctgaacatgaacggcaagaatggtctggcctgtattaccccgatttcggcactcaaccagccgggcaagaagattgtgattcgcccgctgccaggtttaccggtgatccgcgatttggtggtagacatgggacaattctatgcgcaatatgagaaaattaagccttacctgttgaataatggacaaaatccgccagctcgcgagcatttacagatgccagagcagcgcgaaaaactcgacgggctgtatgaatgtattctctgcgcatgttgttcaacctcttgtccgtctttctggtggaatcccgataagtttatcggcccggcaggcttgttagcggcatatcgtttcctgattgatagccgtgataccgagactgacagccgcctcgacggtttgagtgatgcattcagcgtattccgctgtcacagcatcatgaactgcgtcagtgtatgtccgaaggggctgaacccgacgcgcgccatcggccatatcaagtcgatgttgttgcaacgtaatgcgtaaaccgtaggcctgataagacgcgcaagcgtcgcatcaggcaaccagtgccggatgcggcgtgaacgccttatccggcctacaagtcattacccgtaggcctgataag

SEQ ID NO: 16 - DNA, 506 bp, Artificial Sequence
  Synthesized sdhC
gttaacctctgtgcccgtagtccccagggaataataagaacagcatgtgggcgttattcatgataagaaatgtgaaaaaacaaagacctgttaatctggacctacagaccatccggttccccatcacggcgatagcgtccattctccatcgcgtttccggtgtgatcacctttgttgcagtgggcatcctgctgtggcttctgggtaccagcctctcttcccctgaaggtttcgagcaagcttccgcgattatgggcagcttcttcgtcaaatttatcatgtggggcatccttaccgctctggcgtatcacgtcgtcgtaggtattcgccacatgatgatggattttggctatctggaagaaacattcgaagcgggtaaacgctccgccaaaatctcctttgttattactgtcgtgctttcacttctcgcaggagtcctcgtatggtaagcaacgcctccgcattaggacgcaatggcgtacatgatttcatcctcgttcgcgcta

SEQ ID NO: 17 - DNA, 452 bp, Artificial Sequence
  Synthesized sdhD
aaaatctcctttgttattactgtcgtgctttcacttctcgcaggagtcctcgtatggtaagcaacgcctccgcattaggacgcaatggcgtacatgatttcatcctcgttcgcgctaccgctatcgtcctgacgctctacatcatttatatggtcggttttttcgctaccagtggcgagctgacatatgaagtctggatcggtttcttcgcctctgcgttcaccaaagtgttcaccctgctggcgctgttttctatcttgatccatgcctggatcggcatgtggcaggtgttgaccgactacgttaaaccgctggctttgcgcctgatgctgcaactggtgattgtcgttgcactggtggtttacgtgatttatggattcgttgtggtgtggggtgtgtgatgaaattgccagtcagagaatttgatgcagttgtgattggtgccggtggcg

SEQ ID NO: 18 - DNA, 1103 bp, Artificial Sequence
  Synthesized hbd
agttaaagctgctaataattaagataaataaaaagaattatttaaagcttattatgccaaaatacttatatagtattttggtgtaaatgcattgatagtttctttaaatttagggaggtctgtttaatgaaaaaggtatgtgttataggtgcaggtactatgggttcaggaattgctcaggcatttgcagctaaaggatttgaagtagtattaagagatattaaagatgaatttgttgatagaggattagattttatcaataaaaatctttctaaattagttaaaaaaggaaagatagaagaagctactaaagttgaaatcttaactagaatttccggaacagttgaccttaatatggcagctgattgcgatttagttatagaagcagctgttgaaagaatggatattaaaaagcagatttttgctgacttagacaatatatgcaagccagaaacaattcttgcatcaaatacatcatcactttcaataacagaagtggcatcagcaactaaaagacctgataaggttataggtatgcatttctttaatccagctcctgttatgaagcttgtagaggtaataagaggaatagctacatcacaagaaacttttgatgcagttaaagagacatctatagcaataggaaaagatcctgtagaagtagcagaagcaccaggatttgttgtaaatagaatattaataccaatgattaatgaagcagttggtatattagcagaaggaatagcttcagtagaagacatagataaagctatgaaacttggagctaatcacccaatgggaccattagaattaggtgattttataggtcttgatatatgtcttgctataatggatgttttatactcagaaactggagattctaagtatagaccacatacattacttaagaagtatgtaagagcaggatggcttggaagaaaatcaggaaaaggtttctacgattattcaaaataagtttacaagaatccccattatcaaatggggattttttatatataatataattttagaggagggattataatatggattttgatatgatagaagaaaagaaggatagtgttatagtaagaaatgtagaa

SEQ ID NO: 19 - DNA, 862 bp, Artificial Sequence
  Synthesized hbd
ggaggaataattcatgaaaaagatttttgtacttggagcaggaactatgggtgctggtatcgttcaagcattcgctcaaaaaggttgtgaggtaattgtaagagacataaaggaagaatttgttgacagaggaatagctggaatcactaaaggattagaaaagcaagttgctaaaggaaaaatgtctgaagaagataaagaagctatactttcaagaatttcaggaacaactgatatgaagttagctgctgactgtgatttagtagttgaagctgcaatcgaaaacatgaaaattaagaaggaaatctttgctgagttagatggaatttgtaagccagaagcgattttagcttcaaacacttcatctttatcaattactgaagttgcttcagctacaaagagacctgataaagttatcggaatgcatttctttaatccagctccagtaatgaagcttgttgaaattattaaaggaatagctacttctcaagaaacttttgatgctgttaaggaattatcagttgctattggaaaagaaccagtagaagttgcagaagctccaggattcgttgtaaacggaatcttaatcccaatgattaacgaagcttcattcatccttcaagaaggaatagcttcagttgaagatattgatacagctatgaaatatggtgctaaccatccaatgggacctttagctttaggagatcttattggattagatgtttgcttagctatcatggatgttttattcactgaaacaggtgataacaagtacagagctagcagcatattaagaaaatatgttagagctggatggcttggaagaaaatcaggaaaaggattctatgattattctaaataa

SEQ ID NO: 20 - DNA, 1022 bp, Artificial Sequence
  Synthesized crt
taatattgaaatataaaataaatcattatataataattatagaagcatatgcttctaattttttgcccgtctttgttataatattaacaataaaaaaatattttaggaggattagtcatggaactaaacaatgtcatccttgaaaaggaaggtaaagttgctgtagttaccattaacagacctaaagcattaaatgcgttaaatagtgatacactaaaagaaatggattatgttataggtgaaattgaaaatgatagcgaagtacttgcagtaattttaactggagcaggagaaaaatcatttgtagcaggagcagatatttctgagatgaaggaaatgaataccattgaaggtagaaaattcgggatacttggaaataaagtgtttagaagattagaacttcttgaaaagcctgtaatagcagctgttaatggttttgctttaggaggcggatgcgaaatagctatgtcttgtgatataagaatagcttcaagcaacgcaagatttggtcaaccagaagtaggtctcggaataacacctggttttggtggtacacaaagactttcaagattagttggaatgggcatggcaaagcagcttatatttactgcacaaaatataaaggcagatgaagcattaagaatcggacttgtaaataaggtagtagaacctagtgaattaatgaatacagcaaaagaaattgcaaacaaaattgtgagcaatgctccagtagctgttaagttaagcaaacaggctattaatagaggaatgcagtgtgatattgatactgctttagcatttgaatcagaagcatttggagaatgcttttcaacagaggatcaaaaggatgcaatgacagctttcatagagaaaagaaaaattgaaggcttcaaaaatagataggaggtaagtttatatggattttaatttaacaagagaacaagaattagtaagacagatggttagagaatttgctgaaaatgaagttaaacctatagcagcagaaattgatgaaacagaaa

SEQ ID NO: 21 - DNA, 3015 bp, Artificial  Sequence
  Synthesized adhE2
attttactttattctaataatacgtaatacacccacttataactagtatttggcaataaaaatagttataatcattaattattgttaaatgtttgacaatctttaattactgttatataataatattatagaaaataaaatgactgcataattttactatagaaatacaagcgttaaatatgtacatatcaacggtttatcacattagaagtaaataatgtaaggaaaccacactctataatttataaggcatcaaagtgtgttatataatacaataagttttatttgcaatagtttgttaaatatcaaactaataataaattttataaaggagtgtatataaatgaaagttacaaatcaaaaagaactaaaacaaaagctaaatgaattgagagaagcgcaaaagaagtttgcaacctatactcaagagcaagttgataaaatttttaaacaatgtgccatagccgcagctaaagaaagaataaacttagctaaattagcagtagaagaaacaggaataggtcttgtagaagataaaattataaaaaatcattttgcagcagaatatatatacaataaatataaaaatgaaaaaacttgtggcataatagaccatgacgattctttaggcataacaaaggttgctgaaccaattggaattgttgcagccatagttcctactactaatccaacttccacagcaattttcaaatcattaatttctttaaaaacaagaaacgcaatattcttttcaccacatccacgtgcaaaaaaatctacaattgctgcagcaaaattaattttagatgcagctgttaaagcaggagcacctaaaaatataataggctggatagatgagccatcaatagaactttctcaagatttgatgagtgaagctgatataatattagcaacaggaggtccttcaatggttaaagcggcctattcatctggaaaacctgcaattggtgttggagcaggaaatacaccagcaataatagatgagagtgcagatatagatatggcagtaagctccataattttatcaaagacttatgacaatggagtaatatgcgcttctgaacaatcaatattagttatgaattcaatatacgaaaaagttaaagaggaatttgtaaaacgaggatcatatatactcaatcaaaatgaaatagctaaaataaaagaaactatgtttaaaaatggagctattaatgctgacatagttggaaaatctgcttatataattgctaaaatggcaggaattgaagttcctcaaactacaaagatacttataggcgaagtacaatctgttgaaaaaagcgagctgttctcacatgaaaaactatcaccagtacttgcaatgtataaagttaaggattttgatgaagctctaaaaaaggcacaaaggctaatagaattaggtggaagtggacacacgtcatctttatatatagattcacaaaacaataaggataaagttaaagaatttggattagcaatgaaaacttcaaggacatttattaacatgccttcttcacagggagcaagcggagatttatacaattttgcgatagcaccatcatttactcttggatgcggcacttggggaggaaactctgtatcgcaaaatgtagagcctaaacatttattaaatattaaaagtgttgctgaaagaagggaaaatatgctttggtttaaagtgccacaaaaaatatattttaaatatggatgtcttagatttgcattaaaagaattaaaagatatgaataagaaaagagcctttatagtaacagataaagatctttttaaacttggatatgttaataaaataacaaaggtactagatgagatagatattaaatacagtatatttacagatattaaatctgatccaactattgattcagtaaaaaaaggtgctaaagaaatgcttaactttgaacctgatactataatctctattggtggtggatcgccaatggatgcagcaaaggttatgcacttgttatatgaatatccagaagcagaaattgaaaatctagctataaactttatggatataagaaagagaatatgcaatttccctaaattaggtacaaaggcgatttcagtagctattcctacaactgctggtaccggttcagaggcaacaccttttgcagttataactaatgatgaaacaggaatgaaataccctttaacttcttatgaattgaccccaaacatggcaataatagatactgaattaatgttaaatatgcctagaaaattaacagcagcaactggaatagatgcattagttcatgctatagaagcatatgtttcggttatggctacggattatactgatgaattagccttaagagcaataaaaatgatatttaaatatttgcctagagcctataaaaatgggactaacgacattgaagcaagagaaaaaatggcacatgcctctaatattgcggggatggcatttgcaaatgctttcttaggtgtatgccattcaatggctcataaacttggggcaatgcatcacgttccacatggaattgcttgtgctgtattaatagaagaagttattaaatataacgctacagactgtccaacaaagcaaacagcattccctcaatataaatctcctaatgctaagagaaaatatgctgaaattgcagagtatttgaatttaaagggtactagcgataccgaaaaggtaacagccttaatagaagctatttcaaagttaaagatagatttgagtattccacaaaatataagtgccgctggaataaataaaaaagatttttataatacgctagataaaatgtcagagcttgcttttgatgaccaatgtacaacagctaatcctaggtatccacttataagtgaacttaaggatatctatataaaatcattttaaaaaataaagaatgtaaaatagtctttgcttcattatattagcttcatgaagcacatagactattttacattttactcttgttttttatctttcaa

SEQ ID NO: 22 - DNA, 1953 bp, Artificial Sequence
  Synthesized Ald6p
ttgttagtcagctcaaacagcgatttaacggttgagtaacacatcaaaacaccgttcgaggtcaagcctggcgtgtttaacaagttcttgatatcatatataaatgtaataagaagtttggtaatattcaattcgaagtgttcagtcttttacttctcttgttttatagaagaaaaaacatcaagaaacatctttaacatacacaaacacatactatcagaatacaatgactaagctacactttgacactgctgaaccagtcaagatcacacttccaaatggtttgacatacgagcaaccaaccggtctattcattaacaacaagtttatgaaagctcaagacggtaagacctatcccgtcgaagatccttccactgaaaacaccgtttgtgaggtctcttctgccaccactgaagatgttgaatatgctatcgaatgtgccgaccgtgctttccacgacactgaatgggctacccaagacccaagagaaagaggccgtctactaagtaagttggctgacgaattggaaagccaaattgacttggtttcttccattgaagctttggacaatggtaaaactttggccttagcccgtggggatgttaccattgcaatcaactgtctaagagatgctgctgcctatgccgacaaagtcaacggtagaacaatcaacaccggtgacggctacatgaacttcaccaccttagagccaatcggtgtctgtggtcaaattattccatggaactttccaataatgatgttggcttggaagatcgccccagcattggccatgggtaacgtctgtatcttgaaacccgctgctgtcacacctttaaatgccctatactttgcttctttatgtaagaaggttggtattccagctggtgtcgtcaacatcgttccaggtcctggtagaactgttggtgctgctttgaccaacgacccaagaatcagaaagctggcttttaccggttctacagaagtcggtaagagtgttgctgtcgactcttctgaatctaacttgaagaaaatcactttggaactaggtggtaagtccgcccatttggtctttgacgatgctaacattaagaagactttaccaaatctagtaaacggtattttcaagaacgctggtcaaatttgttcctctggttctagaatttacgttcaagaaggtatttacgacgaactattggctgctttcaaggcttacttggaaaccgaaatcaaagttggtaatccatttgacaaggctaacttccaaggtgctatcactaaccgtcaacaattcgacacaattatgaactacatcgatatcggtaagaaagaaggcgccaagatcttaactggtggcgaaaaagttggtgacaagggttacttcatcagaccaaccgttttctacgatgttaatgaagacatgagaattgttaaggaagaaatttttggaccagttgtcactgtcgcaaagttcaagactttagaagaaggtgtcgaaatggctaacagctctgaattcggtctaggttctggtatcgaaacagaatctttgagcacaggtttgaaggtggccaagatgttgaaggccggtaccgtctggatcaacacatacaacgattttgactccagagttccattcggtggtgttaagcaatctggttacggtagagaaatgggtgaagaagtctaccatgcatacactgaagtaaaagctgtcagaattaagttgtaatgtaccaacctgcatttctttccgtcatatacacaaaatactttcatataaacttacttggtcttacgtcataaataaatatgtatacatataaattaaaaaatttggttttatatttttacaaaaagaatcgtttacttcatttctcccttttaagcgatacaatccatgaaaaaagagaaaaagagagaacaggcttgtgccttctttaaaacatcccacac

SEQ ID NO: 23 - DNA, 1237 bp, Artificial Sequence
  Synthesized mhpF
gctgcgcaccggagatatcattcttaccggggcattaggtccgatggtggcggtgaatgcgggcgatcgttttgaagcccatattgaaggcataggttcagttgctgcgacattttcaagcgcagccccaaaaggaagtctgtcatgagtaagcgtaaagtcgccattatcggttctggcaacattggtaccgatctgatgattaaaattttgcgtcacggtcagcatctggagatggcggtgatggttggcattgatcctcagtccgacggtctggcgcgcgccagacgtatgggcgtcgccaccacccatgaaggggtgatcggactgatgaacatgcctgaatttgctgatatcgacattgtatttgatgcgaccagcgccggtgctcatgtgaaaaacgatgccgctttacgcgaagcgaaaccggatattcgcttaattgacctgacgcctgctgccatcggcccttactgcgtgccggtggttaacctcgaggcgaacgtcgatcaactgaacgtcaacatggtcacctgcggcggccaggccaccattccaatggtggcggcagtttcacgcgtggcgcgtgttcattacgccgaaattatcgcttctatcgccagtaaatctgccggacctggcacgcgtgccaatatcgatgaatttacggaaaccacttcccgagccattgaagtggtgggcggcgcggcaaaagggaaggcgattattgtgcttaacccagcagagccaccgttgatgatgcgtgacacggtgtatgtattgagcgacgaagcttcacaagatgatatcgaagcctcaatcaatgaaatggctgaggcggtgcaggcttacgtaccgggttatcgcctgaaacagcgcgtgcagtttgaagttatcccgcaggataaaccggtcaatttaccgggcgtggggcaattctccggactgaaaacagcggtctggctggaagtcgaaggcgcagcgcattatctgcctgcctatgcgggcaacctcgacattatgacttccagtgcgctggcgacagcggaaaaaatggcccagtcactggcgcgcaaggcaggagaagcggcatgaacggtaaaaaactttatatctcggacgtcacattgcgtgacggtatgcacgccattcgtcatcagtattcgctggaaaacgttcgccagattgccaaagcactggacgatgcccgcgtggattcgattgaagtggcccacgg

SEQ ID NO: 24 - DNA, 1000 bp, Artificial Sequence
  Synthesized ldi
cccaggctcgaccggccggccgggtccggttcgctggcctgcacgacggccaggtcgatctccagcgagctcaggtcccgccatgcgcccagatagtccgtgggcaggagctcgacgcgctcgtcctggaagccgggccggaaggcgggcgacatgaaataggcccgctggcgggcccgcggatgcaggccgacataggcgccgtcgttgatgccgggaaagaacacgccgacgaagcgcacgtcggcggccttttccggacgggcgcgcagggcgtcgcggaggaccagcgattcgccggtcatgccggcgaggaacacggtcatgccgggttcgagcgtatcgacgatggcctcggccgagcgtcggggcggcgagccgatcatggccggtccttcaggcgccgaggcggccgatgatggagatggcgcagacgttctggtgcggatgtccgcccaggttctgcgtcagcccgaggtccgccttgggcagctggcgttcgcccgcgcggcccaggatctgctggtagagttcatagaccatgcgcagccccgaggcgccgatggggtggccgaagcacttcaggccgccgtccacctggcaggggatcgcgccgccgcgatcgtagcggccgtccaggatgtcgtagggcgcgcggcccgggtcgctcaggccgaggctttccatcagcaccagctcggtgatcgagaagcagtcgtgcacctcgatcaggtcgagcgcggcgcgcggatcgtcgatgcccgcctcgcggtaggcgcgcgccgcggcgacttcggccgtgcggatcgaggcgccgtcccaggtgttgaactgggcctcttctccgttggagaccgccacctgcagcgccttgacggtgaccatgtcgcgccggccgagccttttcgcggtttccaccgtggtgacgatggcgcaggccgcgccgtcgctcaccccgcagcagtcgtagagccccagcggggcggccaccgtgggcgcc

SEQ ID NO: 25 - DNA, 1732 bp, Artificial Sequence
  Synthesized erg12
gtgtattttatcggttgtaattgtactgacaattttcgggcctcgtttggctgtcgcactgaaatcttcgacagggtatcgaagaacgatacaacgatatgcctataaaggttagttagccatgatattacttcctggttagctggtgcgcttcttagtcctaacttgcaaatttatatctacgtatagaaaactgtcaatatgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcataagctaatttgcgataggcattatttattagttgtttttaatcttaactgtgtatgaagttttatgtaataaagatagaaagagaaacaaaaaaaaatttttcgtagtatcaattcagctttcgaagacagaatgaaatttaagcagaccatagtatccttgatacattgactcataatcagttaaatagccattcaatcc

SEQ ID NO: 26 - DNA, 1762 bp, Artificial Sequence
  Synthesized erg8
aaattgtatacaggaatatgtagatagtaataattattgcgatgcgtttttctatacgagatctaaaaggaaaaaagttcggttttaaggcggagttaacgtcatgtcgaatggaaagaaagattatataaagaatggaaaaacaaaaaaggtgaaaaaacatcttggaaggcctcataataaatcaatcggctgcctcgagaaatgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaataacttaaggtagataatagtggtccatgtgacatctttataaatgtgaagtttgaagtgacagcgcttaacatctaaccattcatcttccgatagtacttgaaattgttcctttcggcggcatgataaaattcttttaatgggtacaagctatacatactaggatgaggatggtactgagaacgataaataaactttctagata

SEQ ID NO: 27 - DNA, 70 bp, Artificial Sequence
  Synthesized coaA
gatagcttcctggcagagattttttcttattattcctccccatctggtgttaccctcctgcccattaacc

SEQ ID NO: 28 - DNA, 70 bp, Artificial Sequence
  Synthesized mgsA
cattcaacagaactgtgacgcgccatggcaaatatcgctttgccgatagagctatgaccgccagaaacat

SEQ ID NO: 29 - DNA, 2109 bp, Artificial Sequence; identical to SEQ ID NO: 1
  Synthesized pckA

SEQ ID NO: 30 - DNA, 2101 bp, Artificial Sequence; identical to SEQ ID NO: 2
  Synthesized pckA

SEQ ID NO: 31 - DNA, 1221 bp, Artificial Sequence; identical to SEQ ID NO: 3
  Synthesized mdh1

SEQ ID NO: 32 - DNA, 1474 bp, Artificial Sequence; identical to SEQ ID NO: 4
  Synthesized mdh2

SEQ ID NO: 33 - DNA, 2141 bp, Artificial Sequence; identical to SEQ ID NO: 5
  Synthesized mqo

SEQ ID NO: 34 - DNA, 1907 bp, Artificial Sequence; identical to SEQ ID NO: 6
  Synthesized fum1

SEQ ID NO: 35 - DNA, 2141 bp, Artificial Sequence; identical to SEQ ID NO: 7
  Synthesized fumA

SEQ ID NO: 36 - DNA, 2141 bp, Artificial Sequence; identical to SEQ ID NO: 8
  Synthesized fumB

SEQ ID NO: 37 - DNA, 1824 bp, Artificial  Sequence; identical to SEQ ID NO: 9
  Synthesized fumC

SEQ ID NO: 38 - DNA, 2351 bp, Artificial Sequence; identical to SEQ ID NO: 10
  Synthesized frdA

SEQ ID NO: 39 - DNA, 955 bp, Artificial Sequence; identical to SEQ ID NO: 11
  Synthesized frdB

SEQ ID NO: 40 - DNA, 514 bp, Artificial Sequence; identical to SEQ ID NO: 12
  Synthesized frdC

SEQ ID NO: 41 - DNA, 468 bp, Artificial Sequence; identical to SEQ ID NO: 13
  Synthesized frdD

SEQ ID NO: 42 - DNA, 2297 bp, Artificial Sequence; identical to SEQ ID NO: 14
  Synthesized sdhA

SEQ ID NO: 43 - DNA, 931 bp, Artificial Sequence; identical to SEQ ID NO: 15
  Synthesized sdhB

SEQ ID NO: 44 - DNA, 452 bp, Artificial Sequence; identical to SEQ ID NO: 17
  Synthesized sdhC

SEQ ID NO: 45 - DNA, 452 bp, Artificial Sequence; identical to SEQ ID NO: 17
  Synthesized sdhD

SEQ ID NO: 46 - DNA, 70 bp, Artificial Sequence
  Synthesized idhA
ctgcgtgattccgtacctatgacgttatcggaagatgagatcgcccgtctcaaaggtattaatgaagatc

SEQ ID NO: 47 - DNA, 70 bp, Artificial Sequence
  Synthesized pct
tctcgttagaagaagttgccgagatctatttacctttgtcacgtttgctgaacttctatataagctcgaa

SEQ ID NO: 48 - DNA, 70 bp, Artificial Sequence
  Synthesized acs1
tctgcgccgtcaggcagttctggaacagtttcttggtaccaacgggcaacgcattccttacattatcagt

SEQ ID NO: 49 - DNA, 70 bp, Artificial Sequence
  Synthesized lcdA
attgctggcagtgtcgcggtggggaaaagtacaaccgcccgtgtattgcaggcgctattaagccgttggc

SEQ ID NO: 50 - DNA, 70 bp, Artificial Sequence; identical to SEQ ID NO: 49
  Synthesized lcdA

SEQ ID NO: 51 - DNA, 70 bp, Artificial Sequence
  Synthesized lcdB
cggaacatcgtcgtgttgaactgatcactacagatggcttccttcaccctaatcaggttctgaaagaacg

SEQ ID NO: 52 - DNA, 70 bp, Artificial Sequence
  Synthesized lcdC
tggtctgatgaagaagaaaggcttcccggaatcgtatgatatgcatcgcctggtgaagtttgtttccgat

SEQ ID NO: 53 - DNA, 1206 bp, Artificial Sequence
  Synthesized paaJ
atgcgtgaagcctttatttgtgacggaattcgtacgccaattggtcgctacggcggggcattatcaagtgttcgggctgatgatctggctgctatccctttgcgggaactgctggtgcgaaacccgcgtctcgatgcggagtgtatcgatgatgtgatcctcggctgtgctaatcaggcgggagaagataaccgtaacgtagcccggatggcgactttactggcggggctgccgcagagtgtttccggcacaaccattaaccgcttgtgtggttccgggctggacgcactggggtttgccgcacgggcgattaaagcgggcgatggcgatttgctgatcgccggtggcgtggagtcaatgtcacgggcaccgtttgttatgggcaaggcagccagtgcattttctcgtcaggctgagatgttcgataccactattggctggcgatttgtgaacccgctcatggctcagcaatttggaactgacagcatgccggaaacggcagagaatgtagctgaactgttaaaaatctcacgagaagatcaagatagttttgcgctacgcagtcagcaacgtacggcaaaagcgcaatcctcaggcattctggctgaggagattgttccggttgtgttgaaaaacaagaaaggtgttgtaacagaaatacaacatgatgagcatctgcgcccggaaacgacgctggaacagttacgtgggttaaaagcaccatttcgtgccaatggggtgattaccgcaggcaatgcttccggggtgaatgacggagccgctgcgttgattattgccagtgaacagatggcagcagcgcaaggactgacaccgcgggcgcgtatcgtagccatggcaaccgccggggtggaaccgcgcctgatggggcttggtccggtgcctgcaactcgccgggtgctggaacgcgcagggctgagtattcacgatatggacgtgattgaactgaacgaagcgttcgcggcccaggcgttgggtgtactacgcgaattggggctgcctgatgatgccccacatgttaaccccaacggaggcgctatcgccttaggccatccgttgggaatgagtggtgcccgcctggcactggctgccagccatgagctgcatcggcgtaacggtcgttacgcattgtgcaccatgtgcatcggtgtcggtcagggcatcgccatgattctggagcgtgtttga

SEQ ID NO: 54 - DNA, 1221 bp, Artificial Sequence
  Synthesized phaD
atgaatgaaccgacccacgccgatgccttgatcatcgacgccgtgcgcacgcccattggccgctatgccggggccctgagcagcgtgcgcgccgacgacctggcggccatcccgctcaaagccttgatccagcgtcaccccgaactggactggaaagccattgatgacgttatcttcggctgtgccaaccaggctggcgaagacaaccgcaacgtggcccacatggcgagcctgctggccgggctgccactcgaagtaccagggaccacgatcaaccgcctgtgcggttccggtctggatgccatcggtaatgcggcacgtgccctgcgctgcggtgaagcggggctcatgctggccggtggtgtggagtccatgtcgcgtgcaccgtttgtgatgggtaagtcggagcaggcattcgggcgtgcggccgagctgttcgacaccaccatcggctggcgtttcgtcaacccgctgatgaaggccgcctacggcatcgattcgatgccggaaacggctgaaaacgtggccgaacagttcggcatctcgcgcgccgaccaggatgcctttgccctgcgcagccagcacaaagccgcagcagctcaggcccgcggccgcctggcgcgggaaatcgtgccggtcgaaatcccgcaacgcaaaggcccagccaaagtggtcgagcatgacgagcacccgcgcggcgacacgaccctggagcagctggctcggctcgggacgccgtttcgtgaaggcggcagcgtaacggcgggtaatgcctccggcgtgaatgacggcgcttgcgccctgctgctggccagcagcgccgcggcccgccgccatgggttgaaggcccgcggccgcatcgtcggcatggcggtggccggggttgagcccaggctgatgggcattggtccggtgcctgcgacccgcaaggtgctggcgctcaccggcctggcactggctgacctggatgtcatcgaactcaatgaggcctttgccgcccaagggctggccgtgttgcgcgagctgggcctggccgacgacgacccgcgagtcaaccgcaacggcggcgccatcgccctgggccatcccctgggcatgagcggtgcccggttggtgaccactgccttgcacgagcttgaagaaacggccggccgctacgccctgtgcaccatgtgcatcggcgtaggccaaggcattgccatgatcatcgagcgcctctga

SEQ ID NO: 55 - DNA, 1212 bp, Artificial Sequence
  Synthesized pcaF
atgacattaaaaaacgcttatatcatcgatgccatccgtactccattcggtcgttatgccggtggccttgcacctgtccgtgcagatgaccttggtgctgtgccgattaaagccctcatgcaacgtaacccaagtgtagattgggaacaggtcgatgatgtgatctatggctgtgccaaccaagccggtgaagataaccgtaatgtcggtcgtatgtcagcacttcttgcaggtttaccatatcaggtaccggcaaccactattaaccgtttatgcggttcttcactcgatgccattgccattgcagcccgtgctattaaagcaggtgaagcgaacttggtgattgcaggtggtgtagaaagcatgagccgtgcgccttatgtaatgggtaagtcagacagtgcttttggccgtagccagaagattgaagacaccaccatgggctggcgttttattaacccaaaacttaaagaattgtatggtgtagacaccatgccccagactgccgaaaacgtggctgaacagtttaacgtcaatcgtgcagatcaggaccagtttgccttggtgagccaacaacgcaccgcaagcgcgcaagccaaaggctttttttctaaagaaatcgtggcagttgaaatccctcagcgtaagggtgatgctgttgtgattgatactgatgaacatccacgtgcatcaaccacccttgaaggtttaagcaaacttaaatctgtggttaaagcagatggcacagtaacagcaggcaatgcttcaggtattaatgatggtgcagcagctctactgattgcttctgatgaagcagttcaggcatacaacctaaaaccccgcgccaagattattgcttcaacagcggtgggtgtagaaccacggattatgggctttgctccagcaccagccattaaaaaattacttaaacaagctaacctgactttagatcagatggatgtaattgagctcaatgaagcttttgctgctcaggctttggcagtgacccgtgatttaggtttgccagatgattctcacaaggtaaacccaaatggtggtgccattgctttgggtcatccacttggtgcttcaggtgcacgcatcgtgactacagccttgaaccagcttgaacaaacaggtggtcgctacgctttgtgttcaatgtgtattggggtgggccaaggcatcgcattgattattgagagagtctaa

SEQ ID NO: 56 - DNA, 1164 bp, Artificial Sequence
  Synthesized fadA
atgaaagacgtagtcattgtcgactgtatccggaccccgatgggccggtccaagggcggcgccttccgcaacgtgcgtgcagaagacttgtccgcgcacctgatgaaatccatcctgctgcgcaaccccaacctcgacccgaacgagatcgaggatatctactggggctgcgtgcagcagaccctggagcagggcttcaacatcgcccgcaacgcagccttgctggccggcattcccaagcaggtgggggcggtcaccgtcaaccgcctgtgcggctccagcatgcaggcgctgcacgatgcctcccgcgccattcaggtaggtgatggggatatcttcatcatcggcggtgtcgagcacatgggccacgtgccgatgagccacggggtggacttccaccccggcatggccaagtcggtggcgaaagcctccggcatgatggggctgaccgccgagatgctcggcaagctgcacggcatcagtcgtcagcagcaggacgagtttgccgcccgctcccatcgtcgcgctcacgccgccaccgtggaaggacgtttcgccaaggagatcgtcgggctggaaggccatgacgccagcggcgcccgcttcttctacgactacgacgaggtgatccgccccgagaccacggtggaaaccctgagccagctgcgcccggtgttcgacccggtcaacggcaccgtcaccgccggcacctcgtcggccctgtccgatggcgccgccgccatgctggtgatgagtgcggaccgcgccaaggcgctcggcctcaccccgcgcgccaagatacgtgccatggccgtcgccggctgcgatgccgccatcatgggttacggcccggtaccggccacccagaaggcgctcaagcgggccggcctgaccatcggcgacatcgacctgttcgagctgaacgaggcgtttgccgcccagtccctgccttgcgtgaaggatctgggtctgcaagacgtggtggatgagaaggtgaacctgaacggcggcgccatcgccctgggtcacccgctcggctgctccggcgcccgcatctccaccaccctgctcaacctgatggaagagaaggacgccaccctgggggttgccaccatgtgcatcggcctgggtcagggcatcgccaccgtgttcgaacgagtgtaa

SEQ ID NO: 57 - DNA, 1005 bp, Artificial Sequence
  Synthesized atoB
atggatattgtgattgttgcggccaagcgtacccccatgggggccttccagggagccttggccaacctgactgcccccgagcttggcgcttgcgccattgctgccgccatagcacaagccgggctcaagggggagcagatcgatgaagcctacatgggcaatgtgctcagtgccggggtggggcaggcacccgcccgtcaggctgtgttgaaggcaggtttgccggagagtgtgccatgcaccactgtcaacaaggtgtgtggttccggcatgaaggcggtgatgctggcggcagacagcttgcgtctgggtgacaccgacatagtgatcgccggtggcatggagagcatgagccgggcgccttacctgctcgacaaggcgcgcagcggttttcgcatggggcatcagagcgtgctggatcatatgttcctcgatggcttgcaggatgcttacgaaggccagttgatggggcattatgcccagttgagtgcggatcgcgccggtctggcccgctccgacatggacgcttttgccatcgcttccctgacgcgtgcgctggctgcccagcagagcggtgctttcaaggccgagctggcccaggttactgtcggtgacaccctgctgctcgccgaggatgagcagcctgccaaggccaggcccgacaagatccctcatctgaaaccggcattcagcaagcagggcaccataacggctgccaatgccagctccatctcggacggagcggcggcgctcatcctgatgcgagccgagacggcggcgcagctgggcctgcctgtgctggccatggcgggttgcaacctgcctcatgacaaggtgaacgtgaacggcggggcctgcgcactggggcatccactgggggcgagtggtgcccgtattctggttacgctcattcatgcactgcatgcgcgcagtctgaaacggggtgtggcaagcctgtgtatcggtggaggggaggcgactgccgtcgccatcgagttgagctaa

SEQ ID NO: 58 - DNA, 1206 bp, Artificial Sequence
  Synthesized pcaF
atgagccgcgaggtattcatctgcgatgccgtgcgcacgccgatcggccgtttcggcggcagtctttccgcggtgcgcgccgacgacctcgcggcggtgccgctgaaggccctggtcgagcgcaacccgggggtcgactggtcggcgctggacgaggtgttcctcggctgcgccaaccaggccggcgaggacaaccgtaacgtggcgcgcatggcgctgctgctggccggtttgccggagagcgtgcccggcgtcaccctcaaccgcctctgcgcctcggggatggacgccatcggcacggcgttccgcgccatcgcctgcggcgagatggagctggccatcgccggcggcgtcgagtcgatgtcgcgcgcgccgtacgtgatgggcaaggccgatagcgccttcgggcgcggccagaagatcgaggacaccaccatcggctggcgcttcgtcaacccgctgatgaaggagcagtacggcatcgacccgatgccgcagaccgccgacaacgtcgccgacgactatcgcgtgtcgcgtgccgaccaggatgccttcgccctgcgcagccagcagcgcgccggcagggcgcaggcggccggtttcttcgccgaggaaatcgtcccggtgacgattcgcgggcgcaagggcgacaccctggtcgagtacgacgagcatccgcgtcccgacaccaccctggaggcgctggcccggctcaagccggtcaacgggccggagaagaccgtcaccgccggcaacgcgtccggggtcaacgacggcgccgccgcgctggtcctggcctccgccgaggcagtggagaagcacggcctgactccgcgcgcgcgggtgctgggcatggccagcgccggcgtcgccccacggatcatgggcatcggcccggtgccggcggtgcgcaagctgctgcggcgcctggacctggcgatcgacgccttcgacgtgatcgaactcaacgaagccttcgccagccagggcctggcctgcctgcgcgaactgggcgtggccgacgacagtgagaaggtcaacccgaacggcggtgccatcgccctcggccacccgctggggatgagcggtgcgcggctggtcctcaccgcgctccatcaacttgagaagagcggcggccggcgcggcctggcgaccatgtgcgtaggcgtcggccaaggcctggcgctggccatcgagcgggtctga

SEQ ID NO: 59 - DNA, 2373 bp, Artificial Sequence
  Synthesized bktB
atgacgcgtgaagtggtagtggtaagcggtgtccgtaccgcgatcgggacctttggcggcagcctgaaggatgtggcaccggcggagctgggcgcactggtggtgcgcgaggcgctggcgcgcgcgcaggtgtcgggcgacgatgtcggccacgtggtattcggcaacgtgatccagaccgagccgcgcgacatgtatctgggccgcgtcgcggccgtcaacggcggggtgacgatcaacgcccccgcgctgaccgtgaaccgcctgtgcggctcgggcctgcaggccattgtcagcgccgcgcagaccatcctgctgggcgataccgacgtcgccatcggcggcggcgcggaaagcatgagccgcgcaccgtacctggcgccggcagcgcgctggggcgcacgcatgggcgacgccggcctggtcgacatgatgctgggtgcgctgcacgatcccttccatcgcatccacatgggcgtgaccgccgagaatgtcgccaaggaatacgacatctcgcgcgcgcagcaggacgaggccgcgctggaatcgcaccgccgcgcttcggcagcgatcaaggccggctacttcaaggaccagatcgtcccggtggtgagcaagggccgcaagggcgacgtgaccttcgacaccgacgagcacgtgcgccatgacgccaccatcgacgacatgaccaagctcaggccggtcttcgtcaaggaaaacggcacggtcacggccggcaatgcctcgggcctgaacgacgccgccgccgcggtggtgatgatggagcgcgccgaagccgagcgccgcggcctgaagccgctggcccgcctggtgtcgtacggccatgccggcgtggacccgaaggccatgggcatcggcccggtgccggcgacgaagatcgcgctggagcgcgccggcctgcaggtgtcggacctggacgtgatcgaagccaacgaagcctttgccgcacaggcgtgcgccgtgaccaaggcgctcggtctggacccggccaaggttaacccgaacggctcgggcatctcgctgggccacccgatcggcgccaccggtgccctgatcacggtgaaggcgctgcatgagctgaaccgcgtgcagggccgctacgcgctggtgacgatgtgcatcggcggcgggcagggcattgccgccatcttcgagcgtatctgaatgaccgaggccgttatcgtttcaaccgcgcgcacgccgatcggcaaggcgtatcgcggcgccctcaacgccaccgagggtgccacactgctcggccacgccatcgagcacgcggtgaagcgcgccggtatcgacccgaaggaggtcgaggacgtggtgatgggcgcggcgatgcagcagggcgccaccggcggcaacatcgcccgcaaggcgctgctgcgcgccggcctgccggtgactaccgccggcaccaccatcgatcggcagtgcgcctccggcctgcaggcgatcgcgctcgccgctcgctcggtgctgttcgacggcgtcgagatcgcggtcggcggtggcggcgagtcgatctcgctcgtccagaacgacaagatgaacaccttccacgccgtcgatccggcgctcgaggcgatcaagggcgacgtctacatggcgatgctcgacaccgccgaaaccgtggcgaagcgctacggcatctcgcgcgagcgccaggacgagtattcgctggaaagccagcgccgcaccgcggctgcgcagcagggcggcaagttcaacgacgagatcgcgccgatctcgaccaagatgggcgtcgtcgacaaggccaccggcgcggtgtcgttcaaggatatcacgctgtcgcaggacgaaggcccgcggccggaaaccaccgctgaaggtctcgccggtcttaaggccgtgcgtggtgaaggcttcaccatcactgccggcaatgccagccagctgtcggacggcgcctcggccacggtgatcatgagcgacaagacggcggccgcgaagggcctcaagccgctcggcatcttccgcggcatggtctcctacggctgcgagccggacgagatgggcatcggcccggtgttcgcggtgccgcgcctgttgaagcgccatggtctcagcgtcgacgacatcggtctgtgggagctgaacgaagccttcgccgtgcaggtgctgtactgccgcgacaagctcggcatcgatccggagaagctcaatgtcaacggcggcgcgatctcggtcggccacccctacggcatgtcgggtgcacgcctcgccggccacgcgctgatcgaaggccgtcgccgcaaggcgaagtacgcggtggtcacgatgtgcgtcggcggcggcatgggctccgccggcctgttcgagatcgtgcactga

SEQ ID NO: 60 - DNA, 1209 bp, Artificial Sequence
  Synthesized phaA
atgcgcagagctgcaatcgtcactcccctccgcacgcccgtcggcaccttcggcggcagcctgcgcccggtgcccgtggaggagctggccgccaccgccgtgcgcgccgtggtggaacgcagcggcatcgatcccgcgcgtatcgatgacgtggtctttgcccagtcctacgccaacagcgaagtgccctgcgtcggccgctgggccgcgctgcaggccggcctgccggtcgaagtgccgggcatgcagctggaccgccgctgcggcggcggcctgcaggccatcgtcacggcctcgatgatggtgcaaagcggcgccgccgacgtggtgatcgcgggcggcgtcgagagcatgagcaatatcgagtactacaccaccgacatgcgctggggcgcgcgctcgggcaatgtgcgcttcttcgaccgcctcgaccgcggccgtgaacgctcccagccggtcgagcgcttcggcaagatctccgggatgatcgagacggccgagaacctggcgcgcgactacggcatcagccgcgaagcggccgatgtcttcgccgcccgcagccacgcacgcgccgcggcagcctgggaggccggccgcttcgatgccgaggtcgtccccgtgcaggtgccccagcgcaagggcgatccggtgcggttcgcgcgcgacgaaggtttccgcccggaaaccacgcgtgaaagcctgggcaagctgcgcacgctgatgccgaacggtaccgtcaccgccggcaacgccagccagcagaacgacgcctcggccgcgtgcctgatcgtggccgaagacaagctggccgaattgggcctcacccccatggcctcgctggtgggctgggcggcggctggctgcgagccctcgcacatgggcatcggcccggtgcccgcggtgaagaagctgctggcgcgcctgaacctgacgctggaccggatggacctggtcgagctgaacgaagccttcgcctgccaggtgctggccgtgctcaagggctgggaatggcatgaccaggacgcgatcgagcagaagctcaacgtgaacggctcgggcatctcgcttggccatccgatcggcgccaccggcgtgcgcatcctggccacgctgctgcacgaactgcagcgccgcggcggccgctatggcctggaaaccatgtgcatcggcggcggccagggtattgccgcggtcttcgaacgctactga

SEQ ID NO: 61 - DNA, 717 bp, Artificial Sequence
  Synthesized Clo1313_1716
atgacattaaagggcaggatacactcatttgaatcttttgggacactggacggaccgggtataagatttgtggttttcatgcagggctgtcccttgcgttgtatatattgccacaacagggatacctgggatgttaatgcggggagtgagtacactccccggcaagtaattgatgaaatgatgaaatacatagactatataaaggtctccggaggcggaataactgttaccggcggggagcctgttctccaggccgattttgtggccgaggtgttcagacttgcaaaagagcagggagtgcatacggcgctggataccaatggatttgctgacatagagaaggttgaaaggcttataaaatacaccgatcttgtattgctggatataaagcatgcccgggaggataaacataagataattaccggtgtgtccaacgaaaaaatcaagcgttttgcgctgtatctttcggaccagggagtgcctatctggataagatatgtccttgtccccggatataccgacgatgaagatgaccttaaaatggcggctgatttcataaaaaagcttaaaacggtggaaaaaatcgaagttcttccttatcacaacatgggagcatacaaatgggaaaaacttggtcagaaatacatgcttgaaggagtaaaggggccgagtgcgcaagaggtggaaaaagcaaagaggattctgtcaggcaaataa

SEQ ID NO: 62 - DNA, 2846 bp, Artificial Sequence
  Synthesized fadB
ggatgatcgtcgagaaaaacattgaacagctcgccggagtgaataagtaacgcatccagcttgaagcgcgccagcgcatcgcgagtccgttcttgtaaggtagctatatgatttttatagagcgaggccagtgattccattttttacccttctgtttttttgaccttaagtctccgcatcttagcacatcgttcatccagagcgtgatttctgccgagcgtgatcagatcggcatttctttaatcttttgtttgcatatttttaacacaaaatacacacttcgactcatctggtacgaccagatcaccttgcggattcaggagactgacatgctttacaaaggcgacaccctgtaccttgactggctggaagatggcattgccgaactggtatttgatgccccaggttcagttaataaactcgacactgcgaccgtcgccagcctcggcgaggccatcggcgtgctggaacagcaatcagatctaaaagggctgctgctgcgttcgaacaaagcagcctttatcgtcggtgctgatatcaccgaatttttgtccctgttcctcgttcctgaagaacagttaagtcagtggctgcactttgccaatagcgtgtttaatcgcctggaagatctgccggtgccgaccattgctgccgtcaatggctatgcgctgggcggtggctgcgaatgcgtgctggcgaccgattatcgtctggcgacgccggatctgcgcatcggtctgccggaaaccaaactgggcatcatgcctggctttggcggttctgtacgtatgccacgtatgctgggcgctgacagtgcgctggaaatcattgccgccggtaaagatgtcggcgcggatcaggcgctgaaaatcggtctggtggatggcgtagtcaaagcagaaaaactggttgaaggcgcaaaggcggttttacgccaggccattaacggcgacctcgactggaaagcaaaacgtcagccgaagctggaaccactaaaactgagcaagattgaagccaccatgagcttcaccatcgctaaagggatggtcgcacaaacagcggggaaacattatccggcccccatcaccgcagtaaaaaccattgaagctgcggcccgttttggtcgtgaagaagccttaaacctggaaaacaaaagttttgtcccgctggcgcataccaacgaagcccgcgcactggtcggcattttccttaacgatcaatatgtaaaaggcaaagcgaagaaactcaccaaagacgttgaaaccccgaaacaggccgcggtgctgggtgcaggcattatgggcggcggcatcgcttaccagtctgcgtggaaaggcgtgccggttgtcatgaaagatatcaacgacaagtcgttaaccctcggcatgaccgaagccgcgaaactgctgaacaagcagcttgagcgcggcaagatcgatggtctgaaactggctggcgtgatctccacaatccacccaacgctcgactacgccggatttgaccgcgtggatattgtggtagaagcggttgttgaaaacccgaaagtgaaaaaagccgtactggcagaaaccgaacaaaaagtacgccaggataccgtgctggcgtctaacacttcaaccattcctatcagcgaactggccaacgcgctggaacgcccggaaaacttctgcgggatgcacttctttaacccggtccaccgaatgccgttggtagaaattattcgcggcgagaaaagctccgacgaaaccatcgcgaaagttgtcgcctgggcgagcaagatgggcaagacgccgattgtggttaacgactgccccggcttctttgttaaccgcgtgctgttcccgtatttcgccggtttcagccagctgctgcgcgacggcgcggatttccgcaagatcgacaaagtgatggaaaaacagtttggctggccgatgggcccggcatatctgctggacgttgtgggcattgataccgcgcatcacgctcaggctgtcatggcagcaggcttcccgcagcggatgcagaaagattaccgcgatgccatcgacgcgctgtttgatgccaaccgctttggtcagaagaacggcctcggtttctggcgttataaagaagacagcaaaggtaagccgaagaaagaagaagacgccgccgttgaagacctgctggcagaagtgagccagccgaagcgcgatttcagcgaagaagagattatcgcccgcatgatgatcccgatggtcaacgaagtggtgcgctgtctggaggaaggcattatcgccactccggcggaagcggatatggcgctggtctacggcctgggcttccctccgttccacggcggcgcgttccgctggctggacaccctcggtagcgcaaaatacctcgatatggcacagcaatatcagcacctcggcccgctgtatgaagtgccggaaggtctgcgtaataaagcgcgtcataacgaaccgtactatcctccggttgagccagcccgtccggttggcgacctgaaaacggcttaaggagtcacaatggaacaggttgtcattgtcgatgcaattcgcaccccgatgggccgttcgaagggcggtgcttttcgtaacgtgcgtgcagaagatctctccgctcatttaatgcgtagcctgctggcgcgtaacccggcgctggaagcggcggccctcgacgatatttactggggttgtgtgcagcagacgctggagcagggttttaatatcgcccgtaacgcggcgctgctggcagaagtaccacactctgtcccggcggttaccgttaatcgcttgtgtggttcatccatgcaggcactgcatgacgcagcacgaatgatcatg

SEQ ID NO: 63 - DNA, 2789 bp, Artificial Sequence
  Synthesized yfcX
cggtttgacgatgagcgatctgacattgatcgatatgcacgaagcctttgcagctcagacgctggcgaatattcagttgctgggtagtgaacgttttgctcgtgaagcactggggcgtgcacatgccactggcgaagtggacgatagcaaatttaacgtgcttggcggttcgattgcttacgggcatcccttcgcggcgaccggcgcgcggatgattacccagacattgcatgaacttcgccgtcgcggcggtggatttggtttagttaccgcctgtgctgccggtgggcttggcgcggcaatggttctggaggcggaataatggaaatgacatcagcgtttacccttaatgttcgtctggacaacattgccgttatcaccatcgacgtaccgggtgagaaaatgaataccctgaaggcggagtttgcctcgcaggtgcgcgccattattaagcaactccgtgaaaacaaagagttgcgaggcgtggtgtttgtctccgctaaaccggacaacttcattgctggcgcagacatcaacatgatcggcaactgcaaaacggcgcaagaagcggaagctctggcgcggcagggccaacagttgatggcggagattcatgctttgcccattcaggttatcgcggctattcatggcgcttgcctgggtggtgggctggagttggcgctggcgtgccacggtcgcgtttgtactgacgatcctaaaacggtgctcggtttgcctgaagtacaacttggattgttacccggttcaggcggcacccagcgtttaccgcgtctgataggcgtcagcacagcattagagatgatcctcaccggaaaacaacttcgggcgaaacaggcattaaagctggggctggtggatgacgttgttccgcactccattctgctggaagccgctgttgagctggcaaagaaggagcgcccatcttcccgccctctacctgtacgcgagcgtattctggcggggccgttaggtcgtgcgctgctgttcaaaatggtcggcaagaaaacagaacacaaaactcaaggcaattatccggcgacagaacgcatcctggaggttgttgaaacgggattagcgcagggcaccagcagcggttatgacgccgaagctcgggcgtttggcgaactggcgatgacgccacaatcgcaggcgctgcgtagtatcttttttgccagtacggacgtgaagaaagatcccggcagtgatgcgccgcctgcgccattaaacagcgtggggattttaggtggtggcttgatgggcggcggtattgcttatgtcactgcttgtaaagcggggattccggtcagaattaaagatatcaacccgcagggcataaatcatgcgctgaagtacagttgggatcagctggagggcaaagttcgccgtcgtcatctcaaagccagcgaacgtgacaaacagctggcattaatctccggaacgacggactatcgcggctttgcccatcgcgatctgattattgaagcggtgtttgaaaatctcgaattgaaacaacagatggtggcggaagttgagcaaaattgcgccgctcataccatctttgcttcgaatacgtcatctttaccgattggtgatatcgccgctcacgccacgcgacctgagcaagttatcggcctgcatttcttcagtccggtggaaaaaatgccgctggtggagattattcctcatgcggggacatcggcgcaaaccatcgctaccacagtaaaactggcgaaaaaacagggtaaaacgccaattgtcgtgcgtgacaaagccggtttttacgtcaatcgcatcttagcgccttacattaatgaagctatccgcatgttgacccaaggtgaacgggtagagcacattgatgccgcgctagtgaaatttggttttccggtaggcccaatccaacttttggatgaggtaggaatcgacaccgggactaaaattattcctgtactggaagccgcttatggagaacgttttagcgcgcctgcaaatgttgtttcttcaattttgaacgacgatcgcaaaggcagaaaaaatggccggggtttctatctttatggtcagaaagggcgtaaaagcaaaaaacaggtcgatcccgccatttacccgctgattggcacacaagggcaggggcgaatctccgcaccgcaggttgctgaacggtgtgtgatgttgatgctgaatgaagcagtacgttgtgttgatgagcaggttatccgtagcgtgcgtgacggggatattggcgcggtatttggcattggttttccgccatttctcggtggaccgttccgctatatcgattctctcggcgcgggcgaagtggttgcaataatgcaacgacttgccacgcagtatggttcccgttttaccccttgcgagcgtttggtcgagatgggcgcgcgtggggaaagtttttggaaaacaactgcaactgacctgcaataagaaggtcaaagctatatgaatccgcgctgaatggcggagtgttggtcaaaatgtaaacgcatattgactatacttacgccattgaggtaaaaaacagcgtttcattcggtgaatggataaggcacaatgccggccaccgttttctttctctggtttcagatgaaagaaaacgggcgaatctggttaacaaaagcggtgcaatatgcaagtttttatcatgcgtcacggcgacgcagccctcgatgccgccagtgattccgttcgtcctctgaccactaatggttgtgacgaatctcgcctgatggcgaactggctgaaaggtc

SEQ ID NO: 64 - DNA, 952 bp, Artificial Sequence
  Synthesized phbB
atggtgatcgagcgggtctaagtcaagccagttggaaagctgagggtgtactccctctcccgcaagcgggagagggagcgagttagcggtagttcttggagtgagcaagagatgtcccttcccgtagccacgctcgtgaccggcggcagttccggtatcggccgcgccatctgtgaaatgctgctggccgatggcgtgacccaggtggtcaatgtcgactatgccgaaccggcctggtcgcacccgaacatgactttcttccaggccgacctgaccgatgccgaggcgacccgcgccgtggccgcgcaggtgacctcgcgcttcgccgtcacgcgcctggtgaacaacgccggcgccacgcgccccggcaccgccgacaccgccaccgttgccgacctggactacgtgaccggcctgcacctgcaagccacgctgctgctgacgcaggcgtgcctgcccgcgatgcgcgccgccggttttggccgcatcgtcaacatggcctcgcgcgccgcgctgggcaaggccgagcgcgtggtgtactccgctaccaaagccggcctgatcggcatgacccgcacgctggcgatggagctgggcggcgacggcgtcaccgtcaacgccgtggctccgggcccgatcgccaccgagctgttccgcaagagcaaccccgaaggtgctgaacagaccaggcgcatcctggccagcatcaccgttaagcgcatgggcacgccggaagacgttgcgcgtgccgcgctgttcttcctgtcgcccgacagcggcttcgtcaccggtcaggtgctgtacgtgtgcggcggcaccacgctgggcgttgcgccggtgtaagcaccgcgcctcggcatccagcatttaagcattcaacaagaagagacgttaaccaagcgtcacgcatggcggcccctggccgggccgcccaccggatgcgcgcatgtgc

SEQ ID NO: 65 - DNA, 979 bp, Artificial Sequence
  Synthesized phaB
ccgccggccggtggcacgtcaccaggagacgccatgcccctgccccacgacccgccagtctgcggcgaagccccgcgcaccgtccggccacaccccatcatgcgaggccgccatgccgacgctgactgaacgcaccgccctcgtcaccggcggcatgggcggcctgggcgaggccatcgccatccgccttcatgcgcagggccaccgggtggctgtcacgcactcgcgggagaacccccacgtcgccgactggctggccgcgcagcaggcgcagggccggaccttcacggcctttcccgtggacgtgggcgactacgacgcctgccagcgatgcgcccggcaggtgctcgatcaggtcggcccggtcgacatcctgatcaacaacgccggcatcacgcaggacatgaccttcaagcgcatgacgcacgaggcctggaagcgcgtgctgaccaccgatctcgactcgctcttcaacatgaccaagccgctgtacgacggcatgctggagcgcggctggggccgcatcgtcaacatctcgtcggtcaacggcgccaagggtgcgttcggccaagccaactatgcggcggccaaggcgggcatccacggcttcaccaagtcgctggcgctggagtgcgcggccaagggcatcactgtgaatacggtatcgccaggctaccttgccacccgcatgacgcgcgacgtgccggccgacatcatggaacagcgcatcctgccgcagatccccgtgggccggctcgggcgcccggacgaagtggcggcgctggtcgccttcctgtgcacggacgacgcggccttcatcaccggagccaacctggccatcaacggcggccagcatatgcaatagcgggacccgcaaaagaaaaggcccgcgccatgcagcgcgggccttcgtgttccgacgcctcatccgtgaggcgtggcgaccgggactcaggcgaagtgattcgcgacgaaatcc

SEQ ID NO: 66 - DNA, 423 bp, Artificial Sequence
  Synthesized Orf1
tcagtccttcggcggttccagatagcgcccgaagcgctcgcgccattcgtcgtcgatcaaggtcgcgcgcggggcgccgccgaggtcggcccacacgaccgtctgcttcgcgcggaagcgcacctgctcgcccatcgacgcggtcgtgacgatgtccatcgagctgccgccgatgcgcgcgacgtagagcgtgaaggtgagctcatcgccgtgcatgctcggtgcgaaaaagtcgacttcgaggtggcgcatcggcacgccgcggcggatctccgcgtgcagcttgtagaagtccacgccgatgccgcggtcgaaccagtcctcgaccacctcattgcacagcaccaggcactgcgggtagaagacgatgccggccgggtcgcagtggtggaaacggatggatttcttgcattcgaagatcat

SEQ ID NO: 67 - DNA, 432 bp, Artificial Sequence
  Synthesized COG0824
tcattgggccgcaacctccaccagccgggtgcgataggcttccaggcgttcgcgcatgggaccgggcatgggaaccgccttcaccttttcctgatcggcgacgacacagacgaaactggtctcgaaggccaccacgccgtcaccccgcgcgccgatggtgcggaaatgaatggaagagccccccaccctgtccaccaggaccgagatatccacccggtcgccgggccgaagcggcgatttgatctccatgccgatcttgacgaagggcgtgccgaagccgtgttccttgttgatggtgtaccagtcatagccgatgacatcggccatgaagacctccagcgcctccatggcgtattccaggaagcggggcgtatagacgatgcgcgccgcgtcggaatcgccgaaatggacccggcggcggtgaatgaacac

SEQ ID NO: 68 - DNA, 411 bp, Artificial Sequence
  Synthesized Jann_0674
atgacccacctctggcccctgcgcgtctactatgaagacgtcgatctggcggggatcgtctactacgccaactacctgaaatacctggagcgggggcgctctgaaatggtgcgtgaggccggcatttcccagctcgacatgaaagctgcggggctggtctttgccgtgcggcgggtggaggcggaatacctcaaacccgccaaatacgatgatgagctggtcgtggagacgcagctggaccgcctgaaaggggccagtttcgacatgccccagcgggtcctgcgcggcgatgacgtgctgctggacgcgcggatcaaggttgtgatcctcaacgcggacggccgggcggcgcgacttccggcggatattcgcgcaaaagtcacagccgtcgcggcaagtgatggcccgtaa

SEQ ID NO: 69 - DNA, 432 bp, Artificial Sequence
  Synthesized SSE37_24444
atgtcgcaggaggaagccgtggggcagccgttcgagcatgagatccgggtgacctggggggactgcgatcccgcgcggatcgcctatacggcgcgcatcccctggttcgcgctggatgcgatcaacgcctggtgggaggagaagctgggcggcggctggttccagatggagctggaccgcggtgtcggcacgccgttcgtcaacatgaccatcgatttccgcagtccggtcacgccgcgccaccggctgctctgcgccgtgcgcccggtgcggctgggcgagacctcggtcagtttcgaagtgctgggacggcaggacggtgtgctgtgtttcgaggggcggttcacctgcgtgttcatcgccgtgccgcgttttcgcaaggcgccgccgccggaggatatccgggcggtggtggaggcgcatctgaactag

SEQ ID NO: 70 - DNA, 414 bp, Artificial Sequence
  Synthesized entH
atgatctggaaacgccatttaacgctcgacgaactgaacgccaccagcgataacacaatggtggcgcatctgggaattgtgtatacccgtctgggcgatgatgtgctggaagccgaaatgccggttgatacccgtactcatcagccgttcggtttactacatggcggcgcgtcggcggcgctggcggaaacgctgggatcgatggccggatttatgatgacccgcgacggacagtgtgtggtaggcacagaacttaatgcaacacaccatcgcccggtgtctgagggaaaggtacgcggcgtctgccagccgctgcatcttggtcggcaaaatcagagctgggaaatcgtcgttttcgatgaacaggggcggcgttgctgcacttgtcggctgggtacggcagttttgggatga

SEQ ID NO: 71 - DNA, 651 bp, Artificial Sequence
  Synthesized atoA
atggatgcgaaacaacgtattgcgcgccgtgtggcgcaagagcttcgtgatggtgacatcgttaacttagggatcggtttacccacaatggtcgccaattatttaccggagggtattcatatcactctgcaatcggaaaacggcttcctcggtttaggcccggtcacgacagcgcatccagatctggtgaacgctggcgggcaaccgtgcggtgttttacccggtgcagccatgtttgatagcgccatgtcatttgcgctaatccgtggcggtcatattgatgcctgcgtgctcggcggtttgcaagtagacgaagaagcaaacctcgcgaactgggtagtgcctgggaaaatggtgcccggtatgggtggcgcgatggatctggtgaccgggtcgcgcaaagtgatcatcgccatggaacattgcgccaaagatggttcagcaaaaattttgcgccgctgcaccatgccactcactgcgcaacatgcggtgcatatgctggttactgaactggctgtctttcgttttattgacggcaaaatgtggctcaccgaaattgccgacgggtgtgatttagccaccgtgcgtgccaaaacagaagctcggtttgaagtcgccgccgatctgaatacgcaacggggtgatttatga

SEQ ID NO: 72 - DNA, 663 bp, Artificial Sequence
  Synthesized atoD
atgaaaacaaaattgatgacattacaagacgccaccggcttctttcgtgacggcatgaccatcatggtgggcggatttatggggattggcactccatcccgcctggttgaagcattactggaatctggtgttcgcgacctgacattgatagccaatgataccgcgtttgttgataccggcatcggtccgctcatcgtcaatggtcgagtccgcaaagtgattgcttcacatatcggcaccaacccggaaacaggtcggcgcatgatatctggtgagatggacgtcgttctggtgccgcaaggtacgctaatcgagcaaattcgctgtggtggagctggacttggtggttttctcaccccaacgggtgtcggcaccgtcgtagaggaaggcaaacagacactgacactcgacggtaaaacctggctgctcgaacgcccactgcgcgccgacctggcgctaattcgcgctcatcgttgcgacacacttggcaacctgacctatcaacttagcgcccgcaactttaaccccctgatagcccttgcggctgatatcacgctggtagagccagatgaactggtcgaaaccggcgagctgcaacctgaccatattgtcacccctggtgccgttatcgaccacatcatcgtttcacaggagagcaaataa

SEQ ID NO: 73 - DNA, 1509 bp, Artificial Sequence
  Synthesized actA
atgtctgatcgcattgcttcagaaaagctgcgctccaagctcatgtccgccgacgaggcggcacagtttgttaaccacggtgacaaggttggtttctccggcttcaccggcgctggctacccaaaggcactgcctacggcaatcgctaaccgggctaaagaagcacacggtgcaggcaacgactacgcaatcgacctgttcactggcgcatcgaccgcccctgactgcgatggcgtacttgcagaagctgacgctatccgctggcgcatgccatacgcatctgatccaatcatgcgtaacaagatcaactccggctccatgggatactccgatatccacctgtcccactccggccagcaggttgaagagggcttcttcggccagctcaacgtagctgtcattgaaatcacccgcatcactgaagagggctacatcatcccttcttcctccgtgggtaacaacgttgagtggctcaacgctgcagagaaggtcatcctcgaggttaactcttggcagtctgaagacctcgaaggtatgcacgacatctggtctgttcctgccctgccaaaccgcattgccgtgccaatcaacaagccaggcgaccgcatcggtaagacctacatcgagttcgacaccgacaaggttgttgctgttgttgagaccaacaccgcagaccgcaacgcaccattcaagcctgtcgacgacatctctaagaagatcgctggcaacttcctcgacttcctggaaagcgaagttgctgcaggtcgcctgtcctacgacggctacatcatgcagtccggcgtgggcaacgtgccaaacgcggtgatggcaggcctgctggaatccaagtttgagaacatccaggcctacaccgaagttatccaggacggcatggtggacctcatcgacgccggcaagatgaccgttgcatccgcaacttccttctccctgtctcctgagtacgcagagaagatgaacaacgaggctaagcgttaccgcgagtccattatcctgcgcccacagcagatctctaaccacccagaggtcatccgccgcgttggcctgatcgccaccaacggtctcatcgaggctgacatttacggcaacgtcaactccaccaacgtttctggctcccgcgtcatgaacggcatcggcggctccggcgacttcacccgtaacggctacatctccagcttcatcaccccttcagaggcaaagggcggcgcaatctctgcgatcgttcctttcgcatcccacatcgaccacaccgagcacgatgtcatggttgttatctctgagtacggttacgcagaccttcgtggtctggctccacgtgagcgcgttgccaagatgatcggcctggctcaccctgattaccgcccactgctcgaggagtactacgctcgcgcaacctccggtgacaacaagtacatgcagacccctcatgatcttgcaaccgcgtttgatttccacatcaacctggctaagaacggctccatgaaggcataa

SEQ ID NO: 74 - DNA, 378 bp, Artificial Sequence
  Synthesized Cg0592
atgaatggtatcggcggctcgggcgatttcacgcgtaacgcctttgcttccacatttatctctccctcggcagccaaagttgatgcgatttccgcgattgtgcctttcgcgtcccatatcgatcacacggaacatgatgcgatggttgtcattactgaatatggctacgcagacctgcgcgggctatcgccaaaacaacgagtccccaaaatgattgccatcgcccacccggactatcgaccactgctggaagcatactttgaccgggcgctgaacagtgctgattcctatcagcacaccctgcatgatctgcgcaccgccttcgatttccataatcgcttgaactcacaaggaaccatgaaaatcgaaaaagcatag

SEQ ID NO: 75 - DNA, 657 bp, Artificial Sequence
  Synthesized ctfA
atgaactctaaaataattagatttgaaaatttaaggtcattctttaaagatgggatgacaattatgattggaggttttttaaactgtggcactccaaccaaattaattgattttttagttaatttaaatataaagaatttaacgattataagtaatgatacatgttatcctaatacaggtattggtaagttaatatcaaataatcaagtaaaaaagcttattgcttcatatataggcagcaacccagatactggcaaaaaactttttaataatgaacttgaagtagagctctctccccaaggaactctagtggaaagaatacgtgcaggcggatctggcttaggtggtgtactaactaaaacaggtttaggaactttgattgaaaaaggaaagaaaaaaatatctataaatggaacggaatatttgttagagctacctcttacagccgatgtagcattaattaaaggtagtattgtagatgaggccggaaacaccttctataaaggtactactaaaaactttaatccctatatggcaatggcagctaaaaccgtaatagttgaagctgaaaatttagttagctgtgaaaaactagaaaaggaaaaagcaatgacccccggagttcttataaattatatagtaaaggagcctgcataa

SEQ ID NO: 76 - DNA, 666 bp, Artificial Sequence
  Synthesized ctfB
atgattaatgataaaaacctagcgaaagaaataatagccaaaagagttgcaagagaattaaaaaatggtcaacttgtaaacttaggtgtaggtcttcctaccatggttgcagattatataccaaaaaatttcaaaattactttccaatcagaaaacggaatagttggaatgggcgctagtcctaaaataaatgaggcagataaagatgtagtaaatgcaggaggagactatacaacagtacttcctgacggcacatttttcgatagctcagtttcgttttcactaatccgtggtggtcacgtagatgttactgttttaggggctctccaggtagatgaaaagggtaatatagccaattggattgttcctggaaaaatgctctctggtatgggtggagctatggatttagtaaatggagctaagaaagtaataattgcaatgagacatacaaataaaggtcaacctaaaattttaaaaaaatgtacacttcccctcacggcaaagtctcaagcaaatctaattgtaacagaacttggagtaattgaggttattaatgatggtttacttctcactgaaattaataaaaacacaaccattgatgaaataaggtctttaactgctgcagatttactcatatccaatgaacttagacccatggctgtttag

SEQ ID NO: 77 - DNA, 1554 bp, Artificial Sequence
  Synthesized Cbei_4543
gtgagaaaagtaaaagttttaacaagtcgcgaagcagtacaaatagtgaaggatggagatgtgttagtaactggcggatttgttggtagttgtgcacctgaaactcttagttgtgctttagaaaaacgtttcattgaaacaaatcatccgcaaaatataactttatttcatgcagcaggacaaggcgatagtaaggggaaaggttcagatcattatgcccacgaaggcttacttaagagagtggttgcaggtcattataatttagcaccgaaaattggaaagttaattaatgaaaataaaatagaagcttataatctaccacaagggacaatttctcaattatttagagatattgcgggaaaaagaattgggacaataactcacgttggattgaatacatttgtggatccaagaattagtggtggaaaattaaatgaaaaaacaaaagaagatctagtaaagctaataaatatagaaggtgaagaaaaattattatacaaatcaattccagttaatgtctgcttcttaagaggatcttttgcagatgaatacggtaatgtatcattagaaaaagaaatagctacacttgaggatacgtcaatagcccaagcttgtaagaataatggcggaaaagtaatagttcaagtagaaaaagtagttgaagcaggatctttagacccacgtcttataaaaattccaggtatatatgtagatgcggttgtaatctcaactcccgaagagcatgaacaatccttcgaatgcccatttaatccagcagtaacaggtgaaatgagaattccattaaacagtgtagaaaaagctccattaaatgagagaaagataattgcgagaagagcagctatggaattaaagaaagatacggtagtaaatttaggtatagggataccagaagttatttctttagttgcgaatgaagaaggaattggtgaatatatgacattaactgtagaagccggtccaataggaggtataccacaaggatgcacagcttttggagcgagtataaatccagaagctattatagatcagccatatcaatttgatttttatgatggtggaggcgtcgatatagcatttttaggactagctcaggttgatgaacatggaaatttgaatgtaagtaagtttgggcctagaattgctggatgtggtggattcataaatataactcaaaatgctaagaaagtgttattttgtggaacattcactgcaggaggcttaaaagtagtaacaggagatggcaaattagaaattaaacaagaaggaaaagctaaaaaattcattaaggatgtagagcaaattacatttagtggagattatgcaagaaggatggatcaacaagttatgtatataactgagagagcagtatttgagttaaggaaagatggattataccttacagaaatagcgcctgggatagatctaaaaaaggatgtattggatttaatggatttcaaacctaaaatggatggagtacctagactaatgaatggaagaatattttatgataagttgatgggattaagggagtaa

SEQ ID NO: 78 - DNA, 654 bp, Artificial Sequence
  Synthesized PcaJ
atgagttatcacaaactgacccgtgaccagatcgcccagcgcgttgcccaagacattccggaaggctcctatgtcaatcttggcattggcctgccgaccaagattgccagctatctgcctgccgacaaagacgtatttctacattcagaaaacggactgctggcctttggcccaccaccagcggccggcgaagaagatccggaactgatcaacgcaggcaaagaatacgtaaccatgctcgaaggcggttgcttctttcaccatggcgactccttcgccatgatgcgcggtggacatctggatatctgcgtattaggcgcattccagatcgccgccaatggagacctggccaactggcacaccggtgcaccggatgccataccgtcggtcggtggagccatggatcttgcggttggggcaaaaaaagtttttgtaaccaccgatcatgtcaccaaaaaaggtgagccgaagattgtagctgaactgacgtatccagccacgggtcagaaatgtgtcgaccggatctacaccgacctgtgcatcatcgatgtggtgccagaaggactgaaagtgatcgagaaagtcgaaggcttaagctttgaagaactacaacgcctgaccggtgcaacactgatcgatgcgacacaaggctaa

SEQ ID NO: 79 - DNA, 1269 bp, Artificial Sequence
  Synthesized oleTJE
atggcaacacttaagagggataagggcttagataatactttgaaagtattaaagcaaggttatctttacacaacaaatcagagaaatcgtctaaacacatcagttttccaaactaaagcactcggtggtaaaccattcgtagttgtgactggtaaggaaggcgctgaaatgttctacaacaatgatgttgttcaacgtgaaggcatgttaccaaaacgtatcgttaatacgctttttggtaaaggtgcaatccatacggtagatggtaaaaaacacgtagacagaaaagcattgttcatgagcttgatgactgaaggtaacttgaattatgtacgagaattaacgcgtacattatggcatgcgaacacacaacgtatggaaagtatggatgaggtaaatatttaccgtgaatctatcgtactacttacaaaagtaggaacacgttgggcaggcgttcaagcaccacctgaagatatcgaaagaatcgcaacagacatggacatcatgatcgattcatttagagcacttggtggtgcctttaaaggttacaaggcatcaaaagaagcacgtcgtcgtgttgaagattggttagaagaacaaattattgagactcgtaaagggaatattcatccaccagaaggtacagcactttacgaatttgcacattgggaagactacttaggtaacccaatggactcaagaacttgtgcgattgacttaatgaacacattccgcccattaatcgcaatcaacagattcgtttcattcggtttacacgcgatgaacgaaaacccaatcacacgtgaaaaaattaaatcagaacctgactatgcatataaattcgctcaagaagttcgtcgttactatccattcgttccattccttccaggtaaagcgaaagtagacatcgacttccaaggcgttacaattcctgcaggtgtaggtcttgcattagatgtttatggtacaacgcatgatgaatcactttgggacgatccaaatgaattccgcccagaaagattcgaaacttgggacggatcaccatttgaccttattccacaaggtggtggagattactggacaaatcaccgttgtgcaggtgaatggatcacagtaatcatcatggaagaaacaatgaaatactttgcagaaaaaataacttatgatgttccagaacaagatttagaagtggacttaaacagtatcccaggatacgttaagagtggctttgtaatcaaaaatgttcgcgaagttgtagacagaacataa

SEQ ID NO: 80 - DNA, 1555 bp, Artificial Sequence
  Synthesized padA1
atgtctgcgcaacctgctcacctgtgtttccgctccttcgtcgaagccctcaaggtcgacaacgaccttgttgaaatcaataccccaattgaccccaatctcgaagctgctgctattacccgccgagtatgtgagaccaacgacaaggctcctttattcaacaacctcatcggcatgaaaaatggcctcttccgtatacttggggctcctggctctctcaggaagtcgtctgctgatcgctacggccgccttgctcgtcacctagccctcccacctacggcctcaatgcgtgagattctcgataagatgctctccgccagcgatatgcctcccatccctccgaccattgttcccaccgggccatgcaaggagaacagcttagatgactctgaattcgaccttaccgaactccccgttcctcttattcacaaatcggatggtggtaaatacatccaaacctatggcatgcacattgtgcagtctccggatggaacctggaccaactggtctattgcccgtgcgatggtccatgacaagaaccatctgaccggcctggttattccccctcagcacatctggcagattcaccagatgtggaagaaggaaggccgcagtgacgttccctgggctttggcctttggtgtcccacccgctgccattatggcctctagcatgcctattcccgatggtgtcaccgaagctgggtacgtgggagctatgacgggatcctccctggagcttgttaaatgtgatacgaacgatctatatgtccccgctacctcagaaatcgttctcgagggcacactctctatcagcgagacaggcccagagggacctttcggtgagatgcatggttacatcttccccggggatactcacctcggcgccaaatacaaggttaaccggatcacctaccgcaacaacgccatcatgcccatgtcttcttgtggccgcttgacggatgaaacggtaagtttagtccctgtcctgccatttatagccaaggactaacacggtctagcacaccatgatcggctctctggctgcggcggagatccgtaagctctgccagcagaatgacctccctatcactgatgccttcgctcctttcgagtctcaagttacctgggttgctctgcgggtcgatactgagaagctacgtgccatgaagacaacgtctgagggattccgcaagagagtgggagacgtcgtcttcaaccacaaggccggatacaccattcatcgtctggtgttggtcggtgacgacattgatgtctatgaaggaaaggatgtgctctgggcgttctccacccgttgccgtcctggtatggacgagactttgtttgaggatgttcgtgggttccccttgattccgtatatgggacacgggaatgggcccgcccaccgcggcggaaaggttgtgtccgacgctcttatgccgactgagtacaccactggtcgcaactgggaggctgctgacttcaaccaatcttatcccgaggatctgaagcagaaggtgttggacaactggacgaagatgggtttcagcaactaa

SEQ ID NO: 81 - DNA, 1680 bp, Artificial Sequence
  Syntheszied sdrA
atgggcaccccgataaatcgtgaagagattgaccgcgtgctgcgaatgaaacgcaatcagcgcgaggctcgagcgtgttatccttgccgccagcgcaaggtgaaatgcgacagcactcagccgtgtcgaacatgtcgccgacgaggccatccccaaatatgtgtgtatgaccaagattcgtctgggtctaaaaaggctcgtagcaccggccaaagacgttcctctgctgcttctcgtggaacaaatcagacaccaaccgccgagcaggcattcgatgccgaaccacaatctctgccctcagcgcgcagtttaccagaagtccagccaaaaacaagacagtactatagtactcgaatcccgtcttccgatggccccgataatgatcttatctactcgggcgacaactcggtattgtcttatttgcgcaaccggacgcaagataccaatggctccatgacccgtgaggtgggctctgttctaggcctgcaaaatacctacggcagttatccatttatggactttcggacaccccaggaccggtggaaggagcttctacgtattattccgcagcgagcggaactgttgaagtaagcacatcttattgttgtttttgataacctctaacggatagcaggttcttccatttctacagaatatcagcttaccctttcaatccgatcatacttgacattgagagatttgagcaagatgtgtgttcatacctcaatgatcttgcagcaggagagctgcagaacacttcaaagatttgcgaacgttgggccactgatcggtctgtcgggctgatcagcctgctacttgcggccttggcttccggtgcgcattattctgacctggattacatgcaaagaacagagctatgccaggattttggtacgtaaccagtatctttacctatgcatgtttgactaaacaggagaagcaaaacgatcctttcaagctcttcgactagccaattttcttttccgtccgacgatggatataatacaagcacttctaatcataggaaacactctgcaaaacaatggccagtctgatgcagcatgggttttgttagggacaacagtccgtctcgcgcagacattaggtcttcacacagaaaagagtgtagcacgcctaccggatcatgtcaaatacaaagcacgaaagctatggtacataaaccatgctacaggtaacgacacaagctgacgcggctacaggtacactgtcgtttggcaagattgcctgctctgtttatgttacgaccggcctcgcgtagtctctatgaccgggtgggctccagattattcaatcctctcgagcagcgaactatctttcacagaagctatgtattttctatgccaaactgccttaaatatgatcacaacagacggaccggagatatcggaaaatgcgcgacagcttgacattttggccacgattgatagcctcaaccaacgcactcagccatatctgcgtgaccgccaggaatgcaaaaccctccaacacaatctggagcacctggcgttacgaatgcacatgtctctagttatttccgtcctgacacgtccagcactgaagcgcactgtaatgcaagacgcgtcctatgacatcttgcgcacccgcgccaaattgagcctgatcgacgcctctagggcctttttggattttcaggctctg

SEQ ID NO: 82 - DNA, 1050 bp, Artificial Sequence
  Synthesizedmvd
ttgaatgatcttaatgtttatggtgaaaaaataagaaatatgcttcttgaacttggcatttataataaatcagatgattattcacctgatattaaatacaataaaacgttccacgcaaatggatacccaataacaggtctttataaattccttggatactatgatagggataataacatagccaactttccatcgatatcgttcacaacgaacttttcatcatgtgatgttacatgcagggtattaagatcaggcaatgacaggatcatattcaacgggaaaaacaatgaaaagtattacaaaagggctgaaaaggccctgtcatttctcaggaaaaaatatagaatagatgcagcatttgagtttaacatcaggataaatagaagatacagggatgccaaaggccttggagaatcggcagccgtggcatcggcaaccgccagggccgttgccgcagcagtctttggcatggatgctgcaaaagacaggggttttgtatcatacctggccaggcatgtctctggctccggtaccagatctgcggcaggaaacctttcaatgtggctttcatatcctggaatagacgatttatcttcaattggcttcgaaataagaaaagacgatttattccatttctatgccataccaatgagatcaagaatagagacattaaatgcacatgattatgcatcctcatcaattttttataatgcatgggtcaaatcaaaattttttgatataatagacatcattgaaaacaaattcaatacaaggatgatgcttgaatactccatgaaggatatgtacaggctgcaggcgcttttaatatcctctggatatatcatatatgaaaagcattatttagacattataagaaaattaagatcatcattaaataactacaaaaacgtttatttcacatctgatacaggaacaagcattgttgttatgtcaacatcaatgaatgagctttcaaggttcgttaacgatcttgatcttgatggtataagcggcaattttccagagaagatcattatagaggaactatga

SEQ ID NO: 83 - DNA, 1191 bp, Artificial Sequence
  Synthesized mvd
atgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataa

SEQ ID NO: 84 - DNA, 1182 bp, Artificial Sequence
  Synthesized mvd
atggacaaaaaggtttatcaatgcaccgttagtgcgcctgttaatattgcagtaattaaatactggggaaagagagatgtggcattgaacttgcctaccaatagctcgatcagtgtgaccctttctcaagatgacttacgtactgttactacagctagttgtagcgagaagtttgagaatgatacactgtggttaaatggaaacgctgaggaaatctttgccaataaacgacttcgtgtctgtgtagaggaactgcgtaaagctagattagatctcgaagaggaaaatgatgatcttgacaagattggtgcattgaagcttcatgtcgtttcagaaaacaacttccctactgctgctggtttggcatcttcagctgctggttatgctgctttttgtgaagcaatcgctagattgtacgatttaccatggacacccactcaattatctcgcattgctagacaggggtctggaagtgcttgtcgtagcttgtttgggggctatgtagcctgggagatgggcgagcttcatagcggtgctgatagtgtagcagttcaagttgaacctgttgaaaattggcccgaaatacgtgttgctgttttagtagcgtccgctgccaaaaaaggggtttcctcaacagctggcatgcaagctacagttgcatcttctaccttgttccaacatcgtattcaaaacatcgttccacaacgtatccaagaaatgaagaccgccattcgtgagcgtgattttgagacttttgcgaagcttaccatgactgattccaatcaattccatgcgtgctgccttgatacttttccccctatcttttacttgaacgatacttcacgtgcggttatccgagttgttgagaatataaatgctactgctggaaagaccattgctgcctatacatttgatgctggcccaaatgctgttatttacttcttggaagaaaactccgagattgtattaaatacactttatgctgttactaaaaatgctgaaggatggagcaagcagtatggctcttcccccgttactgttgattctgctgcagccaatattgtatcatctggtataagccgagttatcttaactcgagtgggtaatgggcctcgagttttgacgattgacgaatctttgatcgatgcatctggcaaccctaaatttataggaagtcattaa

SEQ ID NO: 85 - DNA, 972 bp, Artificial Sequence
  Synthesized mvd
atgaaagcgacagcgacggcccacccgatccaggggctggtgaagtaccacgggatacgcgaccccgaactccggacgccgtatcacgattcgatcagcctctgcactgcgccgagtaactccacgacgacggtcgccttcgaacccgagcgtcccgaggacgagtacgtcatcgacggcgaacacatcgacgggcgcggggccgagcgcatccggaccgtcgtcgataacgttcgcgaacgggccgatctcgacgagcgcgtccgcgtcgcaagtgagaacaacttcccgtcgaacgtcggctttggctcctcggcgtcgggattcgcggcgctggcgactgctctcgttgaggccgctggcctggacctctcacgcccggagatctcgacgattgcccgccgcggctcgacctcggcggcgcgggcggtcacgggtggcttttcggatctgcgggcgggcagtaacgacgccgactgccgttcgaagcgactcgacgtccccttggaggatgacgttcgcatcgtcggcgcagtgattcctgcatacaaagagaccgaggcggcccacgaggaggccgccgagagccacatgttcgagggccgactcgcccacgtccacgagcaactcgcggacatgcgcgacgcgctcggtcgcggggacttcgagcggtccttcgagatcgccgaacacgacacactctcgctggcggcgacgacgatgaccggaccgagcggctgggtctactggcaacccgagagcctcgaagtcttcgagacggttcgggaccttcgcgacgacggggttcccgtctacttctccggggataccggcgcaagcatctacgtcaacaccacggccgagtacgtcgaccgcgtcgaatcggcgatcgaaaccctcgggatcgagacgctcacctggcgcgtcggtggccccgcgcgcgtccgtgatcccgagaaggcactgttctga

SEQ ID NO: 86 - DNA, 984 bp, Artificial Sequence
  Synthesized mvd
atgaaagcgaccgccatggcccacccgattcaggggctggtcaagtatcacgggatgcgagacgagatcgagcgcctgccgtatcacgacagtatcagtctctgtacggccccgagccacactcgcacgaccgtggagttctcgatggactacgaggaggacacgttcgtcgtcgacggcgaggaactcgacggccgggcctacgagcgcgtcgaagccgtcgtcgagaaggctcgttcgaagtccgacgcggcccacaccgtctatccggttcgcctcgagagcgagaacagtttcccgtcgaacgtcgggctgggctcctcttcctcgggcttcgccgccgccgcgatggcgctggccgaggccgccgaactcgacgcctcccgccaggagatttcgacgatcgctcgcgtcggctcggcgtcggccgcccgcgcggtcaccggcgcgttttcgcaactgcacacgggtctgaacgacgaggattgtcgctcgcggcgcatcccgagtgaccttcacgaggacctgaagatcgtcgtcggcctcgtcccctaccacaaggagaccgaggacgcccaccgcgaggccgccgacagccacatgttccaggcccgcaacgcccacatccacggccagatcgccgagatgcgcgacgccctgcggaacaacgagttcgaccgcgccttcgaactcgccgagcaggactccctctcgctggccgcgacgacgatgaccggcccctccgggtgggtctactggcagcccgctaccctgaagatcttcaatacggtgcgggaactccgcgaggaggaggacatccccgtctacttctcgacggacaccggcgccagcgtctacgtcaacaccaccgaggaacacgtcgacgaggtcgaggaggcggtctcggattgcggcgtctccaccaccgtctgggacgtcggcggccctgcgaagctgctagacgaggaaaagcacctgttctag

SEQ ID NO: 87 - DNA, 951 bp, Artificial Sequence
  Synthesized mvd
atgcctacaacagccacagcacatactaatattgcatttattaaatattggggtaaaaaagatgcgcgcttaaatttaccgacaaccagttctttatccctaacactctcacaattttatacaacaacaacagtcacacaaaacaccgacaaagatcaacttgttttaaacggtgagctagccgaccctactagaatacatcattttttaaatacaatacgtgatatccttggtgattttcctgctgtgacagtcacttcagaaaaccatgtgccaaccagtgcaggtctagcctcttcggcttcatctttcgctgcgctaacaggtgcagtaacaagagaaatgggatttgatttgtctaatcaatccttatctcggttagcacgccgtggatctggttccgcctcacgatcgttttacagtcactttgctatctggcatgctggtatggatgatgcctcatcttttgctgaaagtttaaatgcccctgacatgccgattgcccttgtcgttgccgaagtgtccacttcagcaaagaaagtgagctcaagtgatggcatgcaacgtgcaatcacttcaccaaactacgatgattggctcaaccgcagcgcgacacaatttatggatatgcagtctgccattcaacaatcagacatcgaaaaaattggtacgcttgctgaagaaaacgctttagctatgcatgcgcttaatctcactgcacgccataaaccattcacctatttcacgcaagaaacccaacaaatacttgccctagtatcagatttacgacgacaagggatcctagccttcgcaacaatggatgctggtccaaacgtcaaaattataacgactttaaatgatgcaccaaaaattgttacagcactacattctgctttaccatatatccatctcgaaactgctacaagcggatcaggtattacctatgactaa

SEQ ID NO: 88 - DNA, 1005 bp, Artificial Sequence
  Synthesized dvd
atgcgcgcgacacccccgcatcgacgtatgaaagcaaccgcgcgcgcacaccccatccagggcctcgtgaaataccacgggatgcgcgacgagtcgcttcgcatgccgtaccacgactccatcagcgtctgcaccgcgcccagcaacaccacgacgaccgtcgagttcgatcccgaccgcgacgccgaccagtacgtcgtcgacggcgacacggtcaccggtcacggcgcggaccgcatccgcagtgtggtcgatgcggtccgcgaccgcgccgggttcgaccaccgcgtgcgcctggagagccagaacagcttccccacgaacatcggcctggggtcgtcgtcgtcggggttcgcggcggccgcgctggcgtgcgtccgcgccgccggcctggatctggacctcccgacggtgtcgacggtcgcgcgccgcggatcggcgtcggcggcccgcgccgtcacgggcgggttctcggatctgcacgcgggattgaacgacgccgactgccgcagcgaacgcctcgacgcccccgcggagttcgcgtccgatctgcgcatcgtcgtgggcgaagtgcccgcgtacaaggagacggagtctgcccacgccgaggccgccgacagccacatgttcgacgcgcggctggcacacgtccagggccaactcgcggagatgcgtgacgccgtccgcgcgggcgacttccagcgcgtcttcgagaccgccgaacacgactcgctgtcgctcgcggcgacgacgatgacggggccgtccgggtgggtgtactggaagcccgagacgctctcgatattcgagaccgtgcgggagctccgggcggacggcgtgccgacgtacttctcgacggataccggcgcgacagtgtacgtgaacaccactgcgagtcacgccgacgaggtcgaggctgcggtcgccgactgcggcgtcgacaccgccgtctgggaggtcggcgggcctgcccacgaactcgacgagcgcgacgcgatcttctga

SEQ ID NO: 89 - DNA, 1300 bp, Artificial Sequence
  Synthesized dfd
atggcggctgcggactcttcggtctatagggccaccactactgcccctgtcaatattgctgtcatcaagtaagttgactgccccccccccctaaataaaccaaccgcctccttttcttctatcattaaatttgtactaacgctgggacttctctagatactggggaaaacgggacgcaactctgaacctgcccaccaattcttccctctctgtgaccctttcccagcgttcgctccgcaccctcaccaccgcctcctgttctgctatctaccccaccgcagatgagcttatcctcaatggcaagcctcaagatatccaatcctccaagcgtacgctcgcctgtctctccagcctgcgctctcttcgccaggcgctggaatctacagactcatcgttgccgaaattatctacacttcccttgcggattgtttccgagaacaatttccccacggccgctggtcttgctagctcagctgctgggtttgcagccctcgttcgtgctgtagcgaacctctaccaacttccgcaatcacctcgggagctcagccgtatcgctcgtcagggatctggctctgcttgccggtctctgatgggcggctacgtggcttggcgcgctggagagttggaggacggcagcgatagtcttgctgaggaggttgcacctgcctcacactggcctgagatgcgtgccattgtcctggtggtcagcgccgagaagaaggatgtccccagtaccgagggcatgcagacgacggtcgctacctcgagtctcttcgctaccagagcgacatctgttgttcccgagcggatggctgccattgagacagcaatcctgaacaaggactttcctgccttcgccgaactcaccatgcgcgactctaacggcttccacgccacctgccttgactcctggcccccaattttctatatgaacgacgtttcccgggctgctgtcagaattgtccacgatatcaaccgtgctattggccgaactgtgtgtgcgtacacctttgatgctggaccgaatgctgttatctattatctggaaaaggattcggagctggtcgcaggaactgtcaaggcaatcttgaccaccaacactgacggctggaatggtcctttctacgatattctgaaggacgtcactgccccgggtgtttctttggataagattgactctagagccgttgaagttctcaaggagggagtcagccgcgtgattctgaccggtgttggtgagggtcctgtcagtgtagaagaccacctggtcagcgcaactggagatgttctttcgcactaa

SEQ ID NO: 90 - DNA, 954 bp, Artificial Sequence
  Synthesized mvaD
atggatagagagcctgtaacagtacgttcctacgcaaatattgctattatcaaatattggggaaagaaaaaagaaaaagagatagtgcctgctactagcagtatttctctaactttggaaaatatgtatacagagacgaccttgtcgcctttaccagccaatgtaacagctgacgaattttacatcaatgctcagctacaaaatgaggtcgagcatgccaagatgagtaagattattgaccgttatcgtccagctggtgagggctttgtccgtatcgatactcaaaataatatgcctacggcagcgggcctgtcctcaagttctagtggtttgtccgccctggtcaaggcttgtaatgcttatttccagcttggtttgtctcggagtcagttggcacaggaggctaagtttgcctcaggttcttcttctcggagtttttatggaccactaggtgcctgggacaaggatagtgggggaatttaccctgtagagacaaacttgaaactagctatgatcatgttggtgctagaggacaagaaaaaaccaatctctagccgtgacgggatgaaactttgtgtggagacttcgacgacttttgacgactgggttcgtcagtctgagaaggactatcaggatatgctgatttatctcaaggaaaatgactttgccaagattggagaattaacggagaaaaatgctcttgctatgcacgctacgacaaaaacagcatcaccagccttttcttatctgaccgattcatcttatgaagcgatggactttgttcgtcaacttcgcgagcaaggagaggcctgctactttactatggatgccggtcctaatgtcaaagttctttgtcaagagaaagacttggagcatttatcaaaaatcttcggtcaacgttaccgcttgattgtgtcaaaaacaaaggatttgagtcaagatgattgctgttaa

SEQ ID NO: 91 - DNA, 1005 bp, Artificial Sequence
  Synthesized mvaD
atgacaacttatgcacgtgcgcacactaacattgcattgatcaaatattggggcaaagcaaataagcaactgatgctgccggcaaccagcagtatttcgcttaccttgaatgacttttacacggacacggcggtaacttttgaccctgcactcgatcaggatcaattcacgttaaatcaccaaatgcagtcgcctactgctgtcagccgctttttggatcatgttcggcacctggcccaaattgatacacgcgctcgggtcaactcgttgaatcatgtaccgactgctgccggtttggccagttcggcttctgcgtttgcggcactggcactggctacaagtcgcgcggctggcctaaatttaacccctaccgctttgtcacggttggcacgtcgcggctcagggtcggccacccgttcaatctttggcggagcggtaatttggcaccgtggcagcgatgatcaatcctcgtttgccgaacccttaaccattcagccaactctgccgctgcggatgttggtcgtcacggtttccgatcagaaaaaggcagtcagctcccgcaccggcatggccaacacggttgcgaccagcccttattaccaggcatgggtacaatcgaatgaagcgttaatttcacctatgatcacggcattggccgaaaatgatctgacgacgattggtgcactcaccgaattatcgagtatgcgcatgcacgctgccattatggctgaggagccgccgttcacctactttttgccggaaactttacgcgcctggcaattggtgcaagaacaacgggcactcggcattccggcgtttgccacgatggatgccggacccaacgtcaagatcctcacaaccgcaccgtacgtggatgttctcatgaccgccttgcagcctgtttttggcgaccggattttgagcacccgcctcggcccggacgcgcaagtgattacaaaggagcaatttaatgacacagagtcagcaatcacatcgcaaggatga

SEQ ID NO: 92 - DNA, 939 bp, Artificial Sequence
  Synthesized mvaD
atgaaagtaaagtgtaaagccaatgcaagcttggctttaattaaatattggggaaagaaggatgtttttttaaacattccagcgacttctagtcttgctgttagtgttgataaattttattcaataagtgagcttgaactttcagatcgagatgaaataattttaaattcaaagccagttatattgcaaaatagagaaaaggtgttttttgattatgcaagaaaaattcttagtgaaccgaatgttagatttaaaattaaaagtgaaaacaattttccaacagcagcaggccttgcaagttcaagttcaggatttgcttctattgctgcttgtattttgaaatattttgataaatattcttttaatagtgcatctaatcttgcaagagtaggatcagcttctgcagcaagggctatttacggagggtttactattttgaaagaaggttcaaaagaatcttttcaattaagagatgaatcttattttaatgatttgcgcataatatttgccataattgatagtagtgaaaaagaattgtcctcaagagccgcaatgaatatttgcaaacaccatggattttattatgatgcttggattgcttctagtaaaaagatttttaaagatgctttatatttttttttaaaaaaagattttgtgcattttggagcaactattgtaaaaagttatcagaatatgtttgctttaatgtttgcatcttctattttttattttaaaaatagcacaatagatttaattaaatatgccgcttatttaagaaataaaggaattttggtatttgagacaatggatgcgggcccccaagtgaagtttctttgtttggagaaaaatttaaatactattttaaaaggacttaagcagaattttactgacattgagtttattgtttcaaaggttggatgtgacttagaatggatttga

SEQ ID NO: 93 - DNA, 2778 bp, Artificial Sequence
  Synthesized pdc2
atgctttccattcagcaaagatataatatttgtctaatggcggagaggcacccaaagtggacgcaacttgaattggcaaaatgggcttatgagacgttccagctgccaaaaattccatcccaaggcacaatatcgcgtttgttggcaaggaaatcaacttatatgaattgtaaagagcatgaaaaagatgcgaatagattaaggaagccaaataatcttttggttcggaaaattttacaagaatggatttctcaaagtttgtggaatggaatccctataacgtcacctattattcaagacactgcacaagctgtttggcacagaattcctgcggagcatcgcgagggaaatggttcttttagttataaatggatttcgaattttttatcaaaaatggatgtcaatatttctgttttagacgaagagttacccaaaaccccaaaagtctggacatttgaagagagggatgtattgaaggcttatttctccaaaattcctccaaaggatttattcactttagatgaagcgtttctctcctacaacctaccgttggattatgctcaatatgaagcaagtagcattcaaaggcgtatagaggtggcaactgtcatgctgtgctccaatttagatggctctgaaaagttaaaacctgttgtcgtgggcaaatatgatagttacaaatcattcaggaattatttccccaatgaaccgaatgatcctgtgtcacaatcaatgttgggtactaagatggctaagaaatttgatatctcataccatagtaacaggaaagcatggctaacgagcaatcttttccacaactggttagtcaggtgggataagaggttggttgctgtgaataggaagatttggattgttttggatgattcttgctgtcatcgaataattaatttgcgccttcaaaatataaaacttgtatacacttcctcaaattcaaagtttttgccatttaactggggtgtctgggatgaattcaaaacacgatacagaatacaacagtatcaggcgctcattgacttgcaaaatagaatttcgaagaatatccaaaataaaaataaatcagaacggaacgaatgcatacccaatggtaaaaaatgtttgattagctttgagcagagtcaactcacaatgtcaaatgcattcaaatttattaaaaaagcttgggatgatatacccgttgatgctatcaaagcaaattggaaaagttccggtctgcttcctcctgaaatgatacatttgaatgagaatgttagtatggcatttaagaaaaacgaagtcttagagagcgttttgaatagattatgtgatgaatactactgtgttaaaaaatgggaatatgaaatgttgttagatttaaacattgaaaacaaaaacacaaacttcttgagtacagaagaattagtggaaagtgctattgtggagccttgtgaacctgattttgatactgcgccaaaaggtaatgaggtccatgatgataattttgatgtatcagtttttgccaatgaagatgataataatcaaaatcatttaagcatgtcacaagctagccacaaccccgattacaacagtaatcacagcaacaatgctattgaaaatactaataatagaggcagtaataataataacaataataatggtagtagtaataatattaatgataatgatagtagcgtaaagtatttgcaacagaatactgttgataatagtaccaaaacaggtaaccctggacaaccaaatatttctagtatggaatcgcaaaggaactcttcgactacagatttagttgttgacggtaattatgacgtcaattttaacggccttttgaatgatccatataatacaatgaaacagccgggcccattagattataatgtcagtacattaatcgataaacctaatttattcttaagtcctgatttggatttatctactgttggcgttgatatgcaactaccatcatcagaatattttagcgaagtattttcttcagctatcagaaacaacgaaaaagctgcctcagatcagaacaaatcaactgatgaacttccttcaagcacggccatggcaaattcaaactcgataacgactgcccttctagagtcaagaaatcaagcacagccgtttgatgtcccacatatgaatgggttgctgagcgacacatcaaaaagcggacattctgttaattcctcaaatgctatatctcaaaattctctgaataactttcaacataattcggcgtccgtcgcggaagcttcgtctccttcaattacaccatctcctgtggcaataaactcaacaggcgctccagcgagatctattatatctgcacccatagactcaaattcctctgcgtcatcgccatcagctttagaacatcttgaaggtgctgtttccggtatgtcaccctcttccaccacaatattaagtaacttacaaacaaatataaatatcgccaaatcattgagtaccattatgaaacatgcagaatcaaacgaaatatcactgacgaaagaaacaataaatgaacttaatttcaattatttgacacttttaaaaaggattaaaaagactagaaaacaattaaatagcgaaagcattaaaataaacagtaagaatgcacaagaccatttagaaacccttctatctggggctgcagctgcagctgcaacttccgccaataacttggaccttccgactggtggttcaaacctcccagactctaataacttacacttacctggtaacacaggctttttttag

SEQ ID NO: 94 - DNA, 1707 bp, Artificial Sequence
  Synthesized pdc1
atgagttatactgtcggtacctatttagcggagcggcttgtccagattggtctcaagcatcacttcgcagtcgcgggcgactacaacctcgtccttcttgacaacctgcttttgaacaaaaacatggagcaggtttattgctgtaacgaactgaactgcggtttcagtgcagaaggttatgctcgtgccaaaggcgcagcagcagccgtcgttacctacagcgtcggtgcgctttccgcatttgatgctatcggtggcgcctatgcagaaaaccttccggttatcctgatctccggtgctccgaacaacaatgatcacgctgctggtcacgtgttgcatcacgctcttggcaaaaccgactatcactatcagttggaaatggccaagaacatcacggccgcagctgaagcgatttacaccccagaagaagctccggctaaaatcgatcacgtgattaaaactgctcttcgtgagaagaagccggtttatctcgaaatcgcttgcaacattgcttccatgccctgcgccgctcctggaccggcaagcgcattgttcaatgacgaagccagcgacgaagcttctttgaatgcagcggttgaagaaaccctgaaattcatcgccaaccgcgacaaagttgccgtcctcgtcggcagcaagctgcgcgcagctggtgctgaagaagctgctgtcaaatttgctgatgctctcggtggcgcagttgctaccatggctgctgcaaaaagcttcttcccagaagaaaacccgcattacatcggtacctcatggggtgaagtcagctatccgggcgttgaaaagacgatgaaagaagccgatgcggttatcgctctggctcctgtcttcaacgactactccaccactggttggacggatattcctgatcctaagaaactggttctcgctgaaccgcgttctgtcgtcgttaacggcgttcgcttccccagcgttcatctgaaagactatctgacccgtttggctcagaaagtttccaagaaaaccggtgctttggacttcttcaaatccctcaatgcaggtgaactgaagaaagccgctccggctgatccgagtgctccgttggtcaacgcagaaatcgcccgtcaggtcgaagctcttctgaccccgaacacgacggttattgctgaaaccggtgactcttggttcaatgctcagcgcatgaagctcccgaacggtgctcgcgttgaatatgaaatgcagtggggtcacatcggttggtccgttcctgccgccttcggttatgccgtcggtgctccggaacgtcgcaacatcctcatggttggtgatggttccttccagctgacggctcaggaagtcgctcagatggttcgcctgaaactgccggttatcatcttcttgatcaataactatggttacaccatcgaagttatgatccatgatggtccgtacaacaacatcaagaactgggattatgccggtctgatggaagtgttcaacggtaacggtggttatgacagcggtgctggtaaaggcctgaaggctaaaaccggtggcgaactggcagaagctatcaaggttgctctggcaaacaccgacggcccaaccctgatcgaatgcttcatcggtcgtgaagactgcactgaagaattggtcaaatggggtaagcgcgttgctgccgccaacagccgtaagcctgttaacaagctcctctag

SEQ ID NO: 95 - DNA, 2109 bp, Artificial Sequence; identical to SEQ ID NO: 1
  Synthesized pckA

SEQ ID NO: 96 - DNA, 2101 bp, Artificial Sequence; identical to SEQ ID NO: 2
  Synthesized pckA

SEQ ID NO: 97 - DNA, 1221 bp, Artificial Sequence; identical to SEQ ID NO: 3
  Synthesized mdh1

SEQ ID NO: 98 - DNA, 1474 bp, Artificial Sequence; identical to SEQ ID NO: 4
  Synthesized mdh2

SEQ ID NO: 99 - DNA, 2141 bp, Artificial Sequence; identical to SEQ ID NO: 5
  Synthesized mqo

SEQ ID NO: 100 - DNA, 1907 bp, Artificial Sequence; identical to SEQ ID NO: 6
  Synthesized fum1

SEQ ID NO: 101 - DNA, 2141 bp, Artificial Sequence; identical to SEQ ID NO: 7
  Synthesized fumA

SEQ ID NO: 102 - DNA, 2141 bp, Artificial Sequence; identical to SEQ ID NO: 8
  Synthesized fumB

SEQ ID NO: 103 - DNA, 1824 bp, Artificial Sequence; identical to SEQ ID NO: 9
  Synthesized fumC

SEQ ID NO: 104 - DNA, 2351 bp, Artificial Sequence; identical to SEQ ID NO: 10
  Synthesized frdA

SEQ ID NO: 105 - DNA, 955 bp, Artificial Sequence; identical to SEQ ID NO: 11
  Synthesized frdB

SEQ ID NO: 106 - DNA, 514 bp, Artificial Sequence; identical to SEQ ID NO: 12
  Synthesized frdC

SEQ ID NO: 107 - DNA, 468 bp, Artificial Sequence; identical to SEQ ID NO: 13
  Synthesized frdD

SEQ ID NO: 108 - DNA, 2297 bp, Artificial Sequence; identical to SEQ ID NO: 14
  Synthesized sdhA

SEQ ID NO: 109 - DNA, 931 bp, Artificial Sequence; identical to SEQ ID NO: 15
  Synthesized sdhB

SEQ ID NO: 110 - DNA, 506 bp, Artificial Sequence; identical to SEQ ID NO: 16
  Synthesized sdhC

SEQ ID NO: 111 - DNA, 452 bp, Artificial Sequence; identical to SEQ ID NO: 17
  Synthesized sdhD

SEQ ID NO: 112 - DNA, 70 bp, Artificial Sequence; identical to SEQ ID NO: 46
  Synthesized idhA

SEQ ID NO: 113 - DNA, 70 bp, Artificial Sequence; identical to SEQ ID NO: 47
  Synthesized pct

SEQ ID NO: 114 - DNA, 70 bp, Artificial Sequence; identical to SEQ ID NO: 48
  Synthesized acs1

SEQ ID NO: 115 - DNA, 70 bp, Artificial Sequence; identical to SEQ ID NO: 49
  Synthesized lcdA

SEQ ID NO: 116 - DNA, 70 bp, Artificial Sequence; identical to SEQ ID NO: 51
  Synthesized lcdB

SEQ ID NO: 117 - DNA, 70 bp, Artificial Sequence; identical to SEQ ID NO: 52
  Synthesized lcdC

SEQ ID NO: 118 - DNA, 70 bp, Artificial Sequence
  Synthesized Med_2001
ctcaaatccggcgtgccaaacgttacagcacctgtttactcacatcttatttatgatgtgatcccggatg

SEQ ID NO: 119 - DNA, 777 bp, Artificial Sequence
  Synthesized BS732_3413
atgaatgcaatttcacttgcggttgatcaatttgtggcagtcttgacgattcacaatccgccggcaaacgcgctttcaagccggatattagaagagctgtcttcctgtcttgatcaatgtgagacagatgcaggcgtacgaagcattatcattcacggcgagggaagatttttctcagccggcgctgatattaaagaatttacatcgcttaaaggcaatgaggattcctcattgctcgctgagcgcggccagcagttaatggagagaatcgaaagttttccaaaaccgattatcgcagcgattcacggcgctgctctcggaggcgggctggagcttgcaatggcctgtcacatcagaatcgcggcagaagatgctaagctggggctcccggagcttaatctcggtatcattccaggttttgcggggacacagcgccttccgagatatgtcggtacagccaaagcactggagctcatcggatcaggagaaccgatatccggaaaagaagcgcttgatctcggacttgtctcaattggagcgaaagatgaggcagaagtgattgaaaaagcaaaagcacttgccgctaagtttgcagagaaaagcccgcaaacgctggcatctttgcttgaacttctttattccaacaaggtgtattcctatgaaggcagcttaaagcttgaagcaaagcgatttggggaagcatttgagtcagaggacgccaaggaaggcatccaggcatttctcgaaaaaagaaagcctcagttcaaaggcgaataa

SEQ ID NO: 120 - DNA, 1009 bp, Artificial Sequence
  Synthesized CNE_2c24410
ccggcaccgacatcgcgcaaggccttcggcgacctgatccgggcgcagaatgcgcactggaagcaagtcgtcgaggacatccacttcaccaagctccagtaagccacaaggaacgacatgagcgcacttcagcaagaaccgcacgccggcgtcgagatcgacgcgcgcggcatcgccaccgtcaccatccgcgaggcgggctcgctcaatatcctgagcacgccggtgatcgcatcgctcacgcgcgccatcgaagcgctggccggcaatgacgcggtacgcgtgctggtgctgcgcggcgccggcgagaaaggctttgtcggcggtgccgacatcaaggaaatggccgcgctggaccgcgccagcgccgaggccttcatcagcggcctgcgccgcctgtgcgacgccgtgcgccacctgccggtgcccgtgatcgcgcgcatgccgggctggtgcctgggcggtgggctggaactggcgctggcgtgcgatatccgcatcgccgccgacaacgcgcagctgggcatgccggaagtgaaggtcggcatcccgtcggtgatccacgccgcgctgatgccgcgcctgatcggcaatgcgcgcacggcatggatgctgctgacgggcgagatctccgatgccgccgaggcgctgcagtgggggctggtgagccgcgtggtgccactggcggagctggacctggaaatcgaacgcgtggccgcgctgctggccggcttcggcccggtcgcggtgcgtcagcagaagcgcctgctgcgcgagtgggaagatgcgccgctggaagtgtcgatcaacaacagcgtgaccgagttcggcagcgccttcgacaccggcgagccgcagcagcacatggccaccttcctgaaccgcaagcgctgaagcgcggggaagtgcttacggcgccgtggccgcgacggcgccgccttcgcgcgtggccaccacgcgcggatcgttcagcaggaactgcgcaaaggcctcggcaaacgccggcagc

SEQ ID NO: 121 - DNA, 1206 bp, Artificial Sequence; identical to SEQ ID NO: 53
  Synthesized paaJ

SEQ ID NO: 122 - DNA, 1221 bp, Artificial Sequence; identical to SEQ ID NO: 54
  Synthesized phaD

SEQ ID NO: 123 - DNA, 1212 bp, Artificial Sequence; identical to SEQ ID NO: 55
  Synthesized pcaF

SEQ ID NO: 124 - DNA, 1164 bp, Artificial Sequence; identical to SEQ ID NO: 56
  Synthesized fadA

SEQ ID NO: 125 - DNA, 1005 bp, Artificial Sequence; identical to SEQ ID NO: 57
  Synthesized atoB

SEQ ID NO: 126 - DNA, 1206 bp, Artificial Sequence; identical to SEQ ID NO: 58
  Synthesized pcaF

SEQ ID NO: 127 - DNA, 2373 bp, Artificial Sequence; identical to SEQ ID NO: 59
  Synthesized bktB

SEQ ID NO: 128 - DNA, 1209 bp, Artificial Sequence; identical to SEQ ID NO: 60
  Synthesized phaA

SEQ ID NO: 129 - DNA, 717 bp, Artificial Sequence; identical to SEQ ID NO: 61
  Synthesized Clo1313

SEQ ID NO: 130 - DNA, 2846 bp, Artificial Sequence; identical to SEQ ID NO: 62
  Synthesized fadB

SEQ ID NO: 131 - DNA, 2789 bp, Artificial Sequence; identical to SEQ ID NO: 63
  Synthesized yfcX

SEQ ID NO: 132 - DNA, 952 bp, Artificial Sequence; identical to SEQ ID NO: 64
  Synthesized phbB

SEQ ID NO: 133 - DNA, 979 bp, Artificial Sequence; identical to SEQ ID NO: 65
  Synthesized phaB

SEQ ID NO: 134 - DNA, 810 bp, Artificial Sequence
  Syntheized mhpD
atgacgaagcatactcttgagcaactggcggcggatttacgccgcgccgcagagcagggcgaagcgattgcaccgctgcgcgatctgattggtatcgataacgctgaagcggcttacgccattcagcacataaatgtgcaacatgacgttgcgcaggggcgtcgcgtggtagggcgtaaagtgggcctgacacatccgaaagtgcaacaacaactgggcgttgatcaaccggattttgggacgttatttgccgacatgtgttatggcgataacgaaatcattcctttttcccgtgttctgcaaccccgcattgaagcggagatcgcactggtgttgaaccgcgatttgcccgcaaccgatatcaccttcgacgaattgtataacgccattgaatgggtacttccggcgctggaagtggtggggagccgcattcgcgactggtcgattcagtttgtcgataccgtggcagataacgcctcctgtggggtgtatgtcatcggcggtccggcgcaacgtccggcggggttagacctgaaaaactgcgccatgaagatgacgcgtaataacgaagaggtttctagcgggcgcggcagcgaatgcctgggacatccgcttaatgcggccgtctggctggcacgcaaaatggccagtctgggtgaaccgctgcgcaccggagatatcattcttaccggggcattaggtccgatggtggcggtgaatgcgggcgatcgttttgaagcccatattgaaggcataggttcagttgctgcgacattttcaagcgcagccccaaaaggaagtctgtcatga

SEQ ID NO: 135 - DNA, 789 bp, Artificial Sequence
  Synthesized ctmF
atgaatgaagccaacgtgattgcgaacctgttatgggatgcgcagcggcaaaagctgccctgtgcaccggtgcgggaatatttcgaggggaagagcgaggttgaccaggcgctattggcctatgccgtacagcaggtgaatgttcagcgccaggtggagggcggccgacgtatcgtcggtcgcaagatcggccttacctctccggcagtgcagaagcaattgggtgtagatcggccggacttcggcacgttgctggacgacatggcgatcgtcgatggcgagccgatcaacactgcgcgtcttctgcagcccaaggtcgaagctgagatcgccctggtactcgagcgtgacctcgatcgggagcgtcatacagtcgccgacctgatcgacgcgacagcgtatgcacttgctgcaatcgaggtggtggatagccgtatcaccggttggaacatccgctttgttgacaccgtggcagacaacgcctcatcgggcttgttcgtactcggtactcagcctgttggcctgtcgaagcttgatctggccggtatgtcgatgcgcatggcgcgtggcgaagagcttgtatcgcaaggggctggagctgcctgccttggcaacccgttgaacgcagcgcgttggcttgctgacacgttggtccaagtgggcacgccattgcgtgccggcgatgtggttctgaccggcgctctggggccaatggtcgcggtcgagtccggtcacacctatacggcatggatcgatggcttcgccccggtacgagcaattttctcctga

SEQ ID NO: 136 - DNA, 804 bp, Artificial Sequence
  Synthesized hpaH
atgttcgacaaacacacccacaccctgatcgcccagcgtctggatcaggcagaaaaacagcgcgaacagatccgcgcgatctcgctggattacccggagatcaccatcgaagacgcttacgcggtgcagcgtgaatgggttcgactgaaaatcgccgaaggtcgcacgctgaaaggccacaaaatcggcctgacttcgaaagcgatgcaggccagctcgcagatcagcgaaccggattacggtgcactgctggacgacatgttcttccacgatggcagcgatatcccgaccgatcgctttatcgtgccgcgcattgaagtggagctggcttttgtgctggcaaaaccgctgcgtggaccaaactgcacgctgttcgacgtttacaacgccacggactatgtgatcccggcgctggagctgatcgacgctcgctgccacaacatcgatccggaaacccagcgcccgcgtaaagtgttcgacaccatttctgataacgccgccaatgccggggtgatcctcggtggtcgtcccattaagcccgatgagttggatctacgttggatctccgccctgatgtatcgcaatggcgtgattgaagaaaccggcgtcgccgctggcgtgctgaatcatccggcaaacggcgtggcctggctggcgaacaaactcgccccctatgacgtacaactggaagccgggcaaatcattctcggcggttcgttcacccgcccggttccggcgcgtaagggcgacaccttccacgtcgattacggcaacatgggctccattagctgccgctttgtttaa

SEQ ID NO: 137 - DNA, 507 bp, Artificial Sequence
  Synthesized cnbE
atgagaagtataataaagggaagagtttggaagtttggaaataacgtagatacagatgctatattaccagcaaggtatttagtttatacaaaaccagaggaattagctcagtttgttatgactggggcagacccagattttccaaagaaggttaagccaggagatataatagttggaggaaagaactttggatgtggttcaagtagagagcatgccccattaggattaaaaggagctggaatcagctgtgttattgctgagagcttcgcaagaatattttatagaaatgccataaatgttggattaccattaattgaatgtaagggcatttcagagaaagtcaatgaaggggatgagttagaggttaatttagagactggagagattaaaaacttaaccactggagaggttttaaaaggtcaaaaattaccagaattcatgatggaaattttagaggctggaggattaatgccatacttaaagaaaaagatggctgaaagccaataa

SEQ ID NO: 138 - DNA, 1263 bp, Artificial Sequence
  Synthesized dmdA
ttgggtatgacaatgactcagaaaatattggcggcacatgctggtctggaatccgtaaaaccgggtgatttgatcatggcagacctggatctggtgttggggaatgatattacctcaccggtagccatcaatgtttttaaaaatattaataaggaaaccgtttttgacaaagacaaggttgcgctggtcccagaccattttgcgccgaacaaggatattaaggctgcggagcagtgcaaacaggtgcgctgttttgcctgtgagcaggatgtcaccaactattttgaaatcggcgaaatgggtgtagagcatgctctgctgccggaaaagggactggtcgttgccggcgatgtcgtgattggggcagattcgcacacctgtacctatggtgcgcttggggctttctcaaccggtgtgggttctaccgacatggccgttggtatggcaaccggtaaagcctggtttaaggtaccgtctgccattaaattcaatctgactggcgctttcaaagaaggtgtttcaggaaaagacctgattcttcacattatcggaatgattggtgtggatggtgcgctttataaatcaatggaatttgccggagagggtgtgtcaagcctgacgatggatgatcgcttcaccattgcgaatatggccattgaagctggcggtaaaaatggtatcttccctgtcgacgataagaccatcgaatatatgaaggagcattctaccaaggaatacaaggcctttgaagcagacgcagacgccgagtatgacgctgtgtacgatattaatctggcagatatcaagtctacggtagcattcccgcacttgcctgaaaacactaaaaccgttgatgaaattactgaaccggttaagattgaccaggttgttatcggctcatgcaccaatggacgtttctcagactttaaaaaggccgcagatctgatgcgcggtaagcatgttgccaaaggaatccgtgttttgattatcccagcaactcagcagatttacctggattgtatggaagcgggatatttaaaagactttattgaagcgggcgcaacggtgagcacaccgacctgcgggccatgcctgggcggacatatggggattctggcagcgggagaacgctgcgtttccacaacaaaccgtaactttgtcggacgcatgggccatgtggactcggaagtctatctggcgagccccgaggttgcggcggcatctgctatcctgggccgtattgccggaccagaagaattataa

SEQ ID NO: 139 - DNA, 492 bp, Artificial Sequence
  Synthesized dmdB
atgaaagcaaaaggaaaagtatttagatatggcaacaatgttgatacagacgttattattcccgcaagatacctgaacaccagcgatcctctggaattagcggagcattgtatggaggatattgacaaggattttataaaacgcgtggaggacggcgatatcatcgtcgctgatgataattttggctgcggctcttcaagagagcatgcgcccattgccatcaaagcctcaggtgtctcctgtgtaatcgccaatagctttgcgcgtattttttatcgcaattccatcaatatcgggctgccgattctggaatgtccggaagcggtggcagcgattgaagcaggcgacgaagtagaagtggattttgactctggcgttatcactgacgtgaccaagggacagagcttccagggacaggcattccctgaatttatgcagaagctgatcgcagcaggcggcctggtaaattacgtcaacgagaatctcatttag

SEQ ID NO: 140 - DNA, 786 bp, Artificial Sequence
  Synthesized crt
atggaactaaacaatgtcatccttgaaaaggaaggtaaagttgctgtagttaccattaacagacctaaagcattaaatgcgttaaatagtgatacactaaaagaaatggattatgttataggtgaaattgaaaatgatagcgaagtacttgcagtaattttaactggagcaggagaaaaatcatttgtagcaggagcagatatttctgagatgaaggaaatgaataccattgaaggtagaaaattcgggatacttggaaataaagtgtttagaagattagaacttcttgaaaagcctgtaatagcagctgttaatggttttgctttaggaggcggatgcgaaatagctatgtcttgtgatataagaatagcttcaagcaacgcaagatttggtcaaccagaagtaggtctcggaataacacctggttttggtggtacacaaagactttcaagattagttggaatgggcatggcaaagcagcttatatttactgcacaaaatataaaggcagatgaagcattaagaatcggacttgtaaataaggtagtagaacctagtgaattaatgaatacagcaaaagaaattgcaaacaaaattgtgagcaatgctccagtagctgttaagttaagcaaacaggctattaatagaggaatgcagtgtgatattgatactgctttagcatttgaatcagaagcatttggagaatgcttttcaacagaggatcaaaaggatgcaatgacagctttcatagagaaaagaaaaattgaaggcttcaaaaatagatag

SEQ ID NO: 141 - DNA, 70 bp, Artificial Sequence; identical to SEQ ID NO: 49
  Synthesized lcdA

SEQ ID NO: 142 - DNA, 70 bp, Artificial Sequence; identical to SEQ ID NO: 51
  Synthesized lcdB

SEQ ID NO: 143 - DNA, 70 bp, Artificial Sequence; identical to SEQ ID NO: 52
  Synthesized lcdC

SEQ ID NO: 144 - DNA, 423 bp, Artificial Sequence; identical to SEQ ID NO: 66
  Synthesized Orf1

SEQ ID NO: 145 - DNA, 432 bp, Artificial Sequence; identical to SEQ ID NO: 67
  Synthesized COGO824

SEQ ID NO: 146 - DNA, 411 bp, Artificial Sequence; identical to SEQ ID NO: 68
  Synthesized Jann_0674

SEQ ID NO: 147 - DNA, 432 bp, Artificial Sequence; identical to SEQ ID NO: 69
  Synthesized SSE37_24444

SEQ ID NO: 148 - DNA, 414 bp, Artificial Sequence; identical to SEQ ID NO: 70
  Synthesized entH

SEQ ID NO: 149 - DNA, 651 bp, Artificial Sequence; identical to SEQ ID NO: 71
  Synthesized atoA

SEQ ID NO: 150 - DNA, 663 bp, Artificial Sequence; identical to SEQ ID NO: 72
  Synthesized atoD

SEQ ID NO: 151 - DNA, 1509 bp, Artificial Sequence; identical to SEQ ID NO: 73
  Synthesized actA

SEQ ID NO: 152 - DNA, 378 bp, Artificial Sequence; identical to SEQ ID NO: 74
  Synthesized Cg0592

SEQ ID NO: 153 - DNA, 657 bp, Artificial Sequence; identical to SEQ ID NO: 75
  Synthesized ctfA

SEQ ID NO: 154 - DNA, 657 bp, Artificial Sequence; identical to SEQ ID NO: 75
  Synthesized ctfB

SEQ ID NO: 155 - DNA, 1554 bp, Artificial Sequence; identical to SEQ ID NO: 77
  Synthesized Cbei_4543

SEQ ID NO: 156 - DNA, 654 bp, Artificial Sequence; identical to SEQ ID NO: 78
  Synthesized PcaJ

SEQ ID NO: 157 - DNA, 1269 bp, Artificial Sequence; identical to SEQ ID NO: 79
  Synthesized OleTJE

SEQ ID NO: 158 - DNA, 1555 bp, Artificial Sequence; identical to SEQ ID NO: 80
  Synthesized padA1

SEQ ID NO: 159 - DNA, 1680 bp, Artificial Sequence; identical to SEQ ID NO: 81
  Synthesized sdrA

SEQ ID NO: 160 - DNA, 1049 bp, Artificial Sequence
  Synthesized mvd
ttgaatgatcttaatgtttatggtgaaaaaataagaaatatgcttcttgaacttggcatttataataaatcagatgattattcacctgatattaaatacaataaaacgttccacgcaaatggatacccaataacaggtctttataaattccttggatactatgatagggataataacatagccaactttccatcgatatcgttcacaacgaacttttcatcatgtgatgttacatgcagggtattaagatcaggcaatgacaggatcatattcaacgggaaaaacaatgaaaagtattacaaaagggctgaaaaggccctgtcatttctcaggaaaaaatatagaatagatgcagcatttgagtttaacatcaggataaatagaagatacagggatgccaaaggccttggagaatcggcagccgtggcatcggcaaccgccagggccgtgccgcagcagtctttggcatggatgctgcaaaagacaggggttttgtatcatacctggccaggcatgtctctggctccggtaccagatctgcggcaggaaacctttcaatgtggctttcatatcctggaatagacgatttatcttcaattggcttcgaaataagaaaagacgatttattccatttctatgccataccaatgagatcaagaatagagacattaaatgcacatgattatgcatcctcatcaattttttataatgcatgggtcaaatcaaaattttttgatataatagacatcattgaaaacaaattcaatacaaggatgatgcttgaatactccatgaaggatatgtacaggctgcaggcgcttttaatatcctctggatatatcatatatgaaaagcattatttagacattataagaaaattaagatcatcattaaataactacaaaaacgtttatttcacatctgatacaggaacaagcattgttgttatgtcaacatcaatgaatgagctttcaaggttcgttaacgatcttgatcttgatggtataagcggcaattttccagagaagatcattatagaggaactatga

SEQ ID NO: 161 - DNA, 1191 bp, Artificial Sequence; identical to SEQ ID NO: 83
  Synthesized mvd

SEQ ID NO: 162 - DNA, 1182 bp, Artificial Sequence; identical to SEQ ID NO: 84
  Synthesized mvd

SEQ ID NO: 163 - DNA, 972 bp, Artificial Sequence; identical to SEQ ID NO: 85
  Synthesized mvd

SEQ ID NO: 164 - DNA, 972 bp, Artificial Sequence; identical to SEQ ID NO: 85
  Synthesized mvd

SEQ ID NO: 165 - DNA, 951 bp, Artificial Sequence; identical to SEQ ID NO: 87
  Synthesized mvd

SEQ ID NO: 166 - DNA, 1005 bp, Artificial Sequence; identical to SEQ ID NO: 88
  Synthesized dvd

SEQ ID NO: 167 - DNA, 1300 bp, Artificial Sequence; identical to SEQ ID NO: 89
  Synthesized dfd

SEQ ID NO: 168 - DNA, 954 bp, Artificial Sequence; identical to SEQ ID NO: 90
  Synthesized mvaD

SEQ ID NO: 169 - DNA, 1005 bp, Artificial Sequence; identical to SEQ ID NO: 91
  Synthesized mvaD

SEQ ID NO: 170 - DNA, 939 bp, Artificial Sequence; identical to SEQ ID NO: 92
  Synthesized mvaD

SEQ ID NO: 171 - DNA, 2778 bp, Artificial Sequence; identical to SEQ ID NO: 93
  Synthesized pdc2

SEQ ID NO: 172 - DNA, 1707 bp, Artificial Sequence; identical to SEQ ID NO: 94
  Synthesized pdc1

SEQ ID NO: 173 - DNA, 2109 bp, Artificial Sequence; identical to SEQ ID NO: 1
  Synthesized pckA

SEQ ID NO: 174 - DNA, 2101 bp, Artificial Sequence; identical to SEQ ID NO: 2
  Synthesized pckA

SEQ ID NO: 175 - DNA, 1221 bp, Artificial Sequence; identical to SEQ ID NO: 3
  Synthesized mdh1

SEQ ID NO: 176 - DNA, 1474 bp, Artificial Sequence; identical to SEQ ID NO: 4
  Synthesized mdh2

SEQ ID NO: 177 - DNA, 2141 bp, Artificial Sequence; identical to SEQ ID NO: 5
  Synthesized mqo

SEQ ID NO: 178 - DNA, 1907 bp, Artificial Sequence; identical to SEQ ID NO: 6
  Synthesized fum1

SEQ ID NO: 179 - DNA, 2141 bp, Artificial Sequence; identical to SEQ ID NO: 7
  Synthesized fumA

SEQ ID NO: 180 - DNA, 2141 bp, Artificial Sequence; identical to SEQ ID NO: 8
  Synthesized fumB

SEQ ID NO: 181 - DNA, 1824 bp, Artificial Sequence; identical to SEQ ID NO: 9
  Synthesized fumC

SEQ ID NO: 182 - DNA, 2351 bp, Artificial Sequence; identical to SEQ ID NO: 10
  Synthesized frdA

SEQ ID NO: 183 - DNA, 955 bp, Artificial  Sequence; identical to SEQ ID NO: 11
  Synthesized frdB

SEQ ID NO: 184 - DNA, 514 bp, Artificial Sequence; identical to SEQ ID NO: 12
  Synthesized frdC

SEQ ID NO: 185 - DNA, 468 bp, Artificial Sequence; identical to SEQ ID NO: 13
  Synthesized frdD

SEQ ID NO: 186 - DNA, 2297 bp, Artificial Sequence; identical to SEQ ID NO: 14
  Synthesized sdhA

SEQ ID NO: 187 - DNA, 931 bp, Artificial Sequence; identical to SEQ ID NO: 15
  Synthesized sdhB

SEQ ID NO: 188 - DNA, 506 bp, Artificial Sequence; identical to SEQ ID NO: 16
  Synthesized sdhC

SEQ ID NO: 189 - DNA, 452 bp, Artificial Sequence; identical to SEQ ID NO: 17
  Synthesized sdhD

SEQ ID NO: 190 - DNA, 1179 bp, Artificial Sequence
  Synthesized th1A
atgaaagaagttgtaatagctagtgcagtaagaacagcgattggatcttatggaaagtctcttaaggatgtaccagcagtagatttaggagctacagctataaaggaagcagttaaaaaagcaggaataaaaccagaggatgttaatgaagtcattttaggaaatgttcttcaagcaggtttaggacagaatccagcaagacaggcatcttttaaagcaggattaccagttgaaattccagctatgactattaataaggtttgtggttcaggacttagaacagttagcttagcagcacaaattataaaagcaggagatgctgacgtaataatagcaggtggtatggaaaatatgtctagagctccttacttagcgaataacgctagatggggatatagaatgggaaacgctaaatttgttgatgaaatgatcactgacggattgtgggatgcatttaatgattaccacatgggaataacagcagaaaacatagctgagagatggaacatttcaagagaagaacaagatgagtttgctcttgcatcacaaaaaaaagctgaagaagctataaaatcaggtcaatttaaagatgaaatagttcctgtagtaattaaaggcagaaagggagaaactgtagttgatacagatgagcaccctagatttggatcaactatagaaggacttgcaaaattaaaacctgccttcaaaaaagatggaacagttacagctggtaatgcatcaggattaaatgactgtgcagcagtacttgtaatcatgagtgcagaaaaagctaaagagcttggagtaaaaccacttgctaagatagtttcttatggttcagcaggagttgacccagcaataatgggatatggacctttctatgcaacaaaagcagctattgaaaaagcaggttggacagttgatgaattagatttaatagaatcaaatgaagcttttgcagctcaaagtttagcagtagcaaaagatttaaaatttgatatgaataaagtaaatgtaaatggaggagctattgcccttggtcatccaattggagcatcaggtgcaagaatactcgttactcttgtacacgcaatgcaaaaaagagatgcaaaaaaaggcttagcaactttatgtataggtggcggacaaggaacagcaatattgctagaaaagtgctag

SEQ ID NO: 191 - DNA, 1179 bp, Artificial Sequence; identical to SEQ ID NO: 190
  Synthesized Erg10

SEQ ID NO: 192 - DNA, 2792 bp, Artificial Sequence
  Synthesized fdhF
gccagtgcttgccgaagagatgtcattactgatgctggacagccgccgggtgatccaaagcattcagttgatgaaatcgctgggcggcgggtatcaggcaggtcccgtcgtcgagaaaaaataaaatgtctgccgcgtgatggctgtcacgcggtatttcgtttcgtcacgtcaaaactgacgacagcctgtttttcgtcagagttttgaataaatagtgcccgtaatatcagggaatgaccccacataaaatgtggcataaaagatgcatactgtagtcgagagcgcgtatgcgtgatttgattaactggagcgagaccgatgaaaaaagtcgtcacggtttgcccctattgcgcatcaggttgcaaaatcaacctggtcgtcgataacggcaaaatcgtccgggcggaggcagcgcaggggaaaaccaaccagggtaccctgtgtctgaagggttattatggctgggacttcattaacgatacccagatcctgaccccgcgcctgaaaacccccatgatccgtcgccagcgtggcggcaaactcgaacctgtttcctgggatgaggcactgaattacgttgccgagcgcctgagcgccatcaaagagaagtacggtccggatgccatccagacgaccggctcctcgcgtggtacgggtaacgaaaccaactatgtaatgcaaaaatttgcgcgcgccgttattggtaccaataacgttgactgctgcgctcgtgtctgacacggcccatcggttgcaggtctgcaccaatcggtcggtaatggcgcaatgagcaatgctattaacgaaattgataataccgatttagtgttcgttttcgggtacaacccggcggattcccacccaatcgtggcgaatcacgtaattaacgctaaacgtaacggggcgaaaattatcgtctgcgatccgcgcaaaattgaaaccgcgcgcattgctgacatgcacattgcactgaaaaacggctcgaacatcgcgctgttgaatgcgatgggccatgtcattattgaagaaaatctgtacgacaaagcgttcgtcgcttcacgtacagaaggctttgaagagtatcgtaaaatcgttgaaggctacacgccggagtcggttgaagatatcaccggcgtcagcgccagtgagattcgtcaggcggcacggatgtatgcccaggcgaaaagcgccgccatcctgtggggcatgggtgtaacccagttctaccagggcgtggaaaccgtgcgttctctgaccagcctcgcgatgctgaccggtaacctcggtaagccgcatgcgggtgttaacccggttcgtggtcagaacaacgttcagggtgcctgcgatatgggcgcgctgccggatacgtatccgggataccagtacgtgaaagatccggctaaccgcgagaaattcgccaaagcctggggcgtggaaagcctgccagcgcataccggctatcgcatcagcgagctgccgcaccgcgcagcgcatggcgaagtgcgtgccgcgtacattatgggcgaagatccgctacaaactgacgcggagctgtcggcagtacgtaaagcctttgaagatctggaactggttatcgttcaggacatctttatgaccaaaaccgcgtcggcggcggatgttattttaccgtcaacgtcgtggggcgagcatgaaggcgtgtttactgcggctgaccgtggcttccagcgtttcttcaaggcggttgaaccgaaatgggatctgaaaacggactggcaaatcatcagtgaaatcgccacccgtatgggttatccgatgcactacaacaacacccaggagatctgggatgagttgcgtcatctgtgcccggatttctacggtgcgacttacgagaaaatgggcgaactgggcttcattcagtggccttgccgcgatacttcagatgccgatcaggggacttcttatctgtttaaagagaagtttgataccccgaacggtctggcgcagttcttcacctgcgactgggtagcgccaatcgacaaactcaccgacgagtacccgatggtactgtcaacggtgcgtgaagttggtcactactcttgccgttcgatgaccggtaactgtgcggcactggcggcgctggctgatgaacctggctacgcacaaatcaataccgaagacgccaaacgtctgggtattgaagatgaggcattggtttgggtgcactcgcgtaaaggcaaaattatcacccgtgcgcaggtcagcgatcgtccgaacaaaggggcgatttacatgacctaccagtggtggattggtgcctgtaacgagctggttaccgaaaacttaagcccgattacgaaaacgccggagtacaaatactgcgccgttcgcgtcgagccgatcgccgatcagcgcgccgccgagcagtacgtgattgacgagtacaacaagttgaaaactcgcctgcgcgaagcggcactggcgtaataccgtcctttctacagcctcctttcggaggctgtttttttatccattcgaactctttatactggttactccctacccaatcgtattatcaaaatgaaaaaaattatcgcattgatgttgtttttgacattctttgcccacgccaacgactccgagcctggcagccagtatttaaaggcagcagaggccggggaccgacgcgcacaatattttcttgccgacagctggtttagctccggcgatttgagcaaagccgaatattgggcacagaaagccgccgacagcggtgatgctgatgcctgcgcgctgctggcgcagatcaa

SEQ ID NO: 193 - DNA, 1151 bp, Artificial Sequence
  Synthesized fdnH
ggggctttgagggtgtcgcgcgtaaaggttatatcgctaacactctgacgccgaatgtcggtgatgcaaactcgcaaacgccggaatataaagcgttcttagtcaacatcgagaaggcgtaagggggcgaacagatggctatggaaacgcaggacattatcaaaaggtccgcaactaactccatcacgccgccttctcaggtgcgtgattacaaagcagaagtcgcaaaacttatcgacgtttccacctgtatcggctgtaaagcctgtcaggtggcgtgttcggagtggaacgacatccgtgatgaagtggggcactgcgtcggggtttacgataaccccgccgatctgagcgccaagtcctggacggtgatgcgctttagcgaaaccgaacagaacggcaagctggagtggctgatccgtaaagacggctgtatgcactgtgaagatcccggctgcctgaaggcgtgcccgtctgctggtgcaatcattcagtacgctaacgggattgtcgatttccagtcggaaaactgcatcggctgtggttactgcattgccgggtgtccgtttaatattccgcgcctcaacaaagaggataaccgggtatataaatgcacgctctgcgtcgatcgcgtcagcgtcggccaggaaccggcttgtgtgaaaacctgtccgaccggggctatccacttcggcaccaagaaggagatgctggagctggcggaacagcgcgtggcgaaactgaaagcgcgtggttacgaacatgctggcgtctacaacccggaaggggtcggtggtacgcacgttatgtacgtgctgcatcacgccgatcagccggagctgtatcacggtctgccgaaagatccgaagatcgacacctcggtaagcctgtggaaaggcgcgttgaaaccgctggcagcggctggctttattgccacttttgccgggttgattttccactacatcggtattggcccgaataaggaagtggacgatgacgaggaggatcatcatgagtaagtcgaaaatgattgtgcgcaccaaatttattgatcgcgcctgtcactggaccgtggtgatttgcttcttcctggtggcgctgtccgggatttcgttcttcttcccgacgctgcaatggctgacgcaaacctt

SEQ ID NO: 194 - DNA, 1471 bp, Artificial Sequence
  Synthesized fdh1
acaaaattgagtcttttttaatgagttcttgctgaggaaagtttagttaatatatcatttacgtaaaatatgcatattcttgtattgtgctttttttattcattttaagcaggaacaatttacaagtattgcaacgctaatcaaatcaaaataacagctgaaaattaatatgtcgaagggaaaggttttgctggttctttacgaaggtggtaagcatgctgaagagcaggaaaagttattggggtgtattgaaaatgaacttggtatcagaaatttcattgaagaacagggatacgagttggttactaccattgacaaggaccctgagccaacctcaacggtagacagggagttgaaagacgctgaaattgtcattactacgccctttttccccgcctacatctcgagaaacaggattgcagaagctcctaacctgaagctctgtgtaaccgctggcgtcggttcagaccatgtcgatttagaagctgcaaatgaacggaaaatcacggtcaccgaagttactggttctaacgtcgtttctgtcgcagagcacgttatggccacaattttggttttgataagaaactataatggtggtcatcaacaagcaattaatggtgagtgggatattgccggcgtggctaaaaatgagtatgatctggaagacaaaataatttcaacggtaggtgccggtagaattggatatagggttctggaaagattggtcgcatttaatccgaagaagttactgtactacgactaccaggaactacctgcggaagcaatcaatagattgaacgaggccagcaagcttttcaatggcagaggtgatattgttcagagagtagagaaattggaggatatggttgctcagtcagatgttgttaccatcaactgtccattgcacaaggactcaaggggtttattcaataaaaagcttatttcccacatgaaagatggtgcatacttggtgaataccgctagaggtgctatttgtgtcgcagaagatgttgccgaggcagtcaagtctggtaaattggctggctatggtggtgatgtctgggataagcaaccagcaccaaaagaccatccctggaggactatggacaataaggaccacgtgggaaacgcaatgactgttcatatcagtggcacatctctggatgctcaaaagaggtacgctcagggagtaaagaacatcctaaatagttacttttccaaaaagtttgattaccgtccacaggatattattgtgcagaatggttcttatgccaccagagcttatggacagaagaaataagagtgattatgagtatttgtgagcagaagttttccggtctccttttgttcttgttttggcgtattctccactattcgtccatagcacatttataccttagctaaatattttgtaaagcaaaattttcgttatctcttaaaaaatagaagagcggtttattaatatcaaata

SEQ ID NO: 195 - DNA, 2768 bp, Artificial Sequence
  Synthesized CLJU_c06990
aacggtattttggataaaataaaagtaatctttaagattatgatttctaaagagaggttgagagatctctctttttatgtccagcataatgcatctgtatttcattttaatatattttaaaactatatttaaaaagtgattaaaaaatggaactattattttaaattgcaataaagtctcaataaagtattaaaaaaaattaaacaaaagtaaccttagtaaccttgactttaaattctgaatattttaaaatgaaattgtaatcggtaaaagtaatttataggtaaatttaatatataaagttattggggggtaaaacaatggataaaaaagttttaactgtttgtccttactgtggcgctggttgtaatttatacttgcatgtaaagaatggcaaaataattaaagcagagcctgctaatggtaggacaaatgaaggatcactgtgtttaaaaggacactttggttgggattttttaaacgatcctaaaatattgacatctagaattaaacatccgatgataagaaaaaacggagagctagaagaggtaagctgggatgaagctattagttttacggcttcaagattgtcacaaataaaagagaaatatggacctgattccataatgggaacaggatgtgctaggggttctggaaacgaagcaaactacataatgcaaaagtttatgagggcggttattggaaccaataacgtagatcactgtgccagagtttgacatgctccttctgtagccggtctggcttacgttttaggaaatggtgctatgtcaaatggtatacatgaaatagatgatacagatttactacttatttttggatataatggagcagcttcgcatccaatagttgctaagagaatagttagggcaaaacaaaagggtgcaaaggtaatagttgtagatccacgtataacagagtctggtaggatagcagatttatggctccctataaaaaatggaacaaatatggttcttgtaaatacttttgccaacatacttataaataaacagttttataacaaacaatatgtagaagatcatactgttggttttgaagaatatagatctatagttgaaaattatactcctgaatatgcagaaaaagttactggcataccttcagaggatatagtagaagctatgaaaatgtactcaggtgctaaaaatgccatgatattatatggtatgggagtatgtcaatttgctcaagctgtagatgtagttaagggactagcttctatagcattattaactggtaattttggaagacctaatgtaggtataggacctgtaagaggccagaacaatgttcaaggtgcttgtgatatgggagcacttcctaatgtatacccaggttatcaaagtgtaactgacgatgcaattagggaaaaatttgaaaaagcttggggtgttaaacttccaaacaaagttggttatcacctgacacaagttcctgaattaacgcttaaagaggataaaataaaggcatattatataatgggtgaagatccagttcaaagtgatcctgattctaatgaaatgagagagacactggataaaatggaacttgtaatagttcaggatatatttatgaataaaactgcactccatgcagatgtaattttaccttccacgtcttggggagaacatgaaggagtctttagttctgcagatagaggattccagagatttagaaaagctgtagaacctaagggagatgttaaaccagattgggaaataatttcaaaaattgcctgtgctatgggttataatatgcattataacaatactgaggaaatatggaatgaacttataaatttatgtccaaatttcaaaggagcaacttataagagactcgaagaattaggaggaatccaatggccttgtccatctgaaaatcatcctggaacttcttatctctacaaaggtaataaatttaatacacctactggaaaagcaaacttatttgcagcagaatggagacctcctgtagagcagacagataaagattatccacttgttctttctacagttagagaagtaggacattattctgtaagaacaatgacaggaaactgtagggcacttcagcagttagccgatgaaccaggatatgtacaagttaatccaatggatgcaaaggctaagggaataatagatggtgagcttatgagaataagttcacgaagaggttctgtggttgcccgtgcacttattactgaaagggcaaataaaggagcagtctatatgacctatcaatggtgggtaggcgcatgtaatgaacttacatctaataatctagatccagtatcaaaaactcctgaattaaagtattgtgcagtaaaaatagaagctataaaagatcagaaagaagctgaaaagtttataaaagatcaatatgatcttttaaagaaaaagatgaatgtttaatattttaatataaagatggctaaaaagaccttgattaagaggtcttttagccaaaagctttaaatcaataggagttgatagactgtgatagacgtacatgaagcattcaatatagtaatgaataacacaaaactgcttaaaggtgaagatatatcgttgataaattctcttaacagggtattggcagaggatataagctcaaaagataatcttcccccatttgacaaatcctgtatggatgggtatgctttaaaaagtgaagatactaaggaaaaaatgtcaaaatttcgaattaagggaagcataaaggcgggagat

SEQ ID NO: 196 - DNA, 562 bp, Artificial Sequence
  Synthesized CLJU_c07020
caagtgcttcctataacatttaatgaaaatgagaaaagtaagttcttttcgtatattattgattccatggaaattagtataatcaacaatgataatgtttacattcatgtcgataaaatctatgatctaatgatggacaatcttggatattccaagacgtttaaactcactggaggaacacattgtgcagctctttgtgatgaagataaagtaatatctatttgtgaggatgtggctagacacaatgctgtagacaagcttataggtgaggcatttataaaaaatatttatttaaaggataaaataatatttgtgagcagcagagtatcttttgaaatggtatataaaattgctaggctaggggtacctataataatatctaaatctgcacctacaagtctttctatagaatttgcaaaagctttaaatgttacattaattggatttgtaaggggagaaagaatgaatgtatatacaaatccacagagaataatatagtaatttctgttaacatatttttgaatataatcttaaaaaattaatcatatagttatataaaaat

SEQ ID NO: 197 - DNA, 1627 bp, Artificial Sequence
  Synthesized frc
tcgtcctcactacttaccatgcagaaaggaattggaagtggcgatgaaatgattttcagcacttgatacaggattttaaaaatatcgagtgcagctacgacgatattttgcgggttatttatagaagatattacgccttaaataaacgctaaattaacacatatttaattaactaataggtattgctatgtcaactccacttcaaggaattaaagttctcgatttcaccggtgtgcaatctggcccatcttgtactcaaatgctggcctggtttggcgctgacgttattaaaattgaacgtcccggcgttggtgacgtaacgcgtcaccagctgcgagatattcctgatatcgatgcgctttacttcaccatgcttaacagtaacaaacgttctattgagttaaataccaaaacagcggaaggcaaagaggtaatggaaaagctgatccgcgaagctgatatcttagtcgagaactttcatccaggggccattgatcacatgggcttcacctgggagcatattcaagaaatcaatccacgtctgatttttggttcgatcaaagggtttgatgagtgttcgccttatgtgaatgtaaaagcctatgaaaacgttgctcaggcagcgggtggcgcggcatccactacgggtttttgggatggtccgccgctggtaagcgctgcagcgttgggtgacagcaacaccggaatgcatttgctgatcggtttacttgctgctttgctgcatcgcgaaaaaacggggcgtgggcaacgagtcaccatgtcaatgcaggatgccgtattgaacctttgccgcgtgaaattacgtgaccagcagcgtctcgataaattgggttatctggaagaatacccgcagtatccgaatggtacatttggtgatgcagttccccgcggtggtaatgcaggtggtggcggtcagcctggctggatcctgaaatgtaaaggctgggaaaccgatcctaacgcctatatttatttcactattcaggagcaaaactgggaaaacacctgtaaagccatcggcaaaccagaatggattaccgatccggcatacagtacagcccatgcacgacagccacatattttcgatatttttgctgaaatcgaaaaatacactgtcactattgataaacatgaagcggtggcctatttgactcagtttgatattccttgtgcaccggttttaagtatgaaagaaatttcacttgatccctctttgcgccaaagtggcagtgttgttgaagtggaacaaccgttgcgtggaaaatatctgaccgttggttgtccaatgaaattctctgcctttacgccggatattaaagctgcgccgctattaggtgaacataccgctgctgtattgcaggagctgggttatagcgacgatgaaattgctgcaatgaagcaaaaccacgccatctgatattaacgggggcttctggctcccaatttataaaaatttcgaggttattaatcatgtcagatcaacttcaaatgacagatggtatgcatatcatcgttgaagcattaaaacagaataatattgacactatttatggtgttgtaggtattcctgtgacggatatggcacgccatgcccaggcggaaggca

SEQ ID NO: 198 - DNA, 1627 bp, Artificial Sequence
  Synthesized frc
tcgtcctcaccacttaccatgcagaaaggaattggaagtggcgataaaataattttcagtacttgatacaggaatttaaaaatatcgagtgccgctacgaagatattttgcagattatttatagagggcgttatgccttaaataaacgctaaattaacacatatttaattaactaataggtattgctatgtcaactccacttcaaggaattaaagttctcgatttcaccggtgtgcaatctggcccatcttgtactcaaatgctggcctggtttggcgctgacgttattaaaattgaacgtcccggcgttggtgacgtaacgcgtcaccaactgcgagatattcctgatatcgatgcgctttacttcaccatgcttaacagtaacaaacgttctattgagttaaataccaaaacagcggaaggcaaagaggtaatggaaaagctgatccgcgaagctgatatcttagtcgagaactttcatccaggggccattgatcacatgggcttcacctgggagcatattcaagaaatcaatccacgtctgatttttggttcgatcaaagggtttgatgagtgttcgccttatgtgaatgtaaaagcctatgaaaacgttgctcaggcagcgggtggcgcggcatccactacgggtttttgggatggtccgccgctggtaagcgctgcagcgttgggtgacagcaacaccggaatgcatttgctgatcggtttacttgctgctttgctgcatcgcgaaaaaacggggcgtgggcaacgagtcaccatgtcaatgcaggatgccgtattgaacctttgccgcgtgaaattacgtgaccagcagcgtctcgataaattgggttatctggaagaatacccgcagtatccgaatggtacatttggtgatgcagttccccgcggtggtaatgcaggtggtggcggtcagcctggctggatcctgaaatgtaaaggctgggaaaccgatcctaacgcctatatttatttcactattcaggagcaaaactgggaaaacacctgtaaagccatcggcaaaccagaatggattaccgatccggcatacagtacagcccatgcccgacagccacatattttcgatatttttgctgaaatcgaaaaatacactgtcactattgataaacatgaagcggtggcctatttgactcagtttgatattccttgtgcaccggttttaagtatgaaagaaatttcacttgatccctctttgcgccaaagtggcagtgttgttgaagtggaacaaccgttgcgtggaaaatatctgaccgttggttgtccaatgaaattctctgcctttacgccggatattaaagctgcgccgctattaggtgaacataccgctgctgtattgcaggagctgggttatagcgacgatgaaattgctgcaatgaagcaaaaccacgccatctgatattaacgggggcttctggctcccaatttataaaaatttcgaggttattaatcatgtcagatcaacttcaaatgacagatggtatgcatatcatcgttgaagcattaaaacagaataatattgacactatttatggtgttgtaggtattcctgtgacggatatggcacgccatgcccaggcggaaggca

SEQ ID NO: 199 - DNA, 1598 bp, Artificial Sequence
  Synthesized frc
acgacaagttcgcccagatgctgggcggttacggcgaggaggtccgcgaccccgccgacatcggcccggcgctgcggcgcgcccgcgagtcgggtaagccgtcgctgatcaacgtctgggtcgacccggacgcgtacgcccccggaaccatgaaccagacgatgtacaagtgaggtgagccgaccatgaccaaggctctcgaaggcatccgcgtcctcgacatgacgcacgtccagtccgggccctccgcgacccagctgctcgcctggctcggcgcggacgtggtcaaactggaggcgccgaccggtgacatcacgcgggggcagctgcgcgacctcccggacgtcgactccctctatttcacgatgctcaactgcaacaagcggagcatcaccctcaacaccaagaccgagcgcggcaaggagatcctcaccgagctgatccggcgctccgacgtcatggtcgagaacttcggaccgggcgcggtcgaccgtatgggcttcacctgggaccgcattcgggacatcaatccgcggatcgtctatgcctccatcaaggggttcggcgacgggccgtacaccgacttcaaggcgtacgaggtcgtcgcgcaggccatgggcgggtcgatgtcgaccaccggcttcgaggacgggccgccgctggcgacgggggcccagatcggggactcgggcacgggcatccacgccgtggcggggattctcgcggcgctgtaccagcgggagaacaccgggcgcggtcagcgggtcaacgtggccatgcagcatgctgtgctcaacctctgccgggtgaagctgcgcgatcagcagcgcctggcccatggcccgctcgctgaatatcccaacgacgacttcggcgacgaggttccccggtccgggaacgcgtccggcggcggccagcccggctgggcggtcaagtgcgcgccgggcggccccaacgactacgtgtacgtcatcgtgcagcccgtcggctggaagccgctcagcgagctcatcggccggcccgagctggcggacgaccccgagtgggcgacgccggaggcgcggctgccccagctcaccaagatgttccagctcatcgaggaatggtcggccacgctccccaagtgggaggtgctggagaagctcaacgcccacaacatcccgtgcggccccatcctgtccaccaaggagatcgtcgaggacgcgtcgctggtcgccaacgagatggtggtgacggtgccgcatccggagcgcggcgagttcgtgaccgtgggcagcccgctgaagctctccgactcccccgtggacgtgaccagctcgcccctgctcggcgagcacaacgcagaggtgtacgtcggcgaactcggcctcggggacgaggagctgcgcctgctcaagtcgaacggagtgatctgacgtgatggccgaagaccgggcgctgagggtgcggacgctcctcgacaccgtgcgggccgagggacggaccgcgctgacggcgcccgagggcaaggtgatcgccgacgcgtacgggatcgccgtgcccggcgaggaactggcgacggacgtcgacgaagcggtcgcgtacgcggcgcgcttcgg

SEQ ID NO: 200 - DNA, 1657 bp, Artificial Sequence
  Synthesized frc
ctgcgatggactccggcaagccgaccctcatcaacgcggtcattgaccccgcggcgggcaccgagagcggcaacattggcagcctcaacccgcagagcgttgtcgtgaggaagaagtaagagatcaactccggcgccgtccgaaagacggcgtcgggttcagcggccgataagaaacaactggaggaaagttatggcaaaggcgctcaacggtgttcggattcttgatttcacgcacgtgcaatcgggtccgacgtgtactcaattgctcgcgtggttcggcgcggatgtgatcaaggttgagcgtcccggcaccggcgacatcacccgtggacagctgcaggacgttccgaacgcggacagcctctacttcacgatgctgaaccacaacaagcgctcgatcaccctcgatgcgaagaaccccaagggcaaggaagttctgacagcgctcatcaagagctgcgatgtgatggtcgaaaacttcgcccccggcgtgctcgaccgcatgggcttctcctgggagaacattcagaagatcaacccgaagctgatcgttgcctcgatcaagggcttcggtccgggcccgttcgaagactgcaaggtctacgagaacgtcgcgcagtgcaccggcggttcggcttcgaccacgggcttccgtgatggtctgccgctcgtgactggcgcccagattggcgacagcggcaccggcctccacctcgctctcggtatcgtcaccgctcttttccaccgcacccactcgggcaagggccagcgcgtcaccgtcgcgatgcaggacagcgtgctcaacctctgccgcgtcaagatgcgcgaccagcagcgtctcgctcacggcccgctcaaggaatacagccagttcggcgaaggcattccgttcggcgatgccaccccgcgcgccggcaacgactcgggtggcggccagccgggccgtatcctgaagtgcaagggctgggagaccgacccgaacgcttacatctatttcatcacgcaggcggctgtgtgggagaagatctgcgacgtcatcggtgagccgacctggaagaccgacccgaactacgccaagccgggtgcccgtctgccgcgtctgaacgagatcttcggccgcatcgagcagtggaccatgaccaagaccaagttcgaagtcatgaacatctgcaacccgttcgacattccgtgcggcccgatcctgtcgatgaaggaaatcgccgaggacaagtcgctgtacgcgaccggcacgctggtcgaggtcgatcatccgacccgcggcaagtacatctcggtcggcaacccgatcaagctgtccgacagcccggcggacgttcgccgctcgccgctgctcggtgagcacaccgacgaaatccttcgcgacgtgttgaagttctccgagagccaggtgtcggacatccgttcgtccggcgctctcggcgaagttccgctcgccgcagagtaattacctctcccccgaggatgaccttgccggggcttagccccggcatttttttgtctccatcgtgctgcgatgcaatattgccgacagcttttatgccggtacatgacgagggcatcagtagcctcgtcatcaaatgcaactgccgccattttcatggcggcagttttgttttgggcgaaggcgatcaggc

SEQ ID NO: 201 - DNA, 1724 bp, Artificial Sequence
  Synthesized frc
gcccgagcatctggcgatgatcgaggcgctggaaacacgcgacaccgaactcgccgaaaagctggcgcgcgatcacacgctgcggctggctgcttacgtcgagacccacggccaggagctgttcgcctgacgcggcgacgcttggccgacgcgacataacaaggacgttcgtcgcggatcgccgcggcggacgtaccgatgggagagatgccgcttcggcgcgcaagagacaacaggagcacgaccatgaccaaggcgctcgacggcgttcgcgttctcgacttcacccacgtccaatccggcccgacctgcacgcagctcttggcgtggttcggtgccgacgtgatcaaggtggagcgccccggcagcggcgacatcacccgcggtcagctgcaggacatcccgaaggtggacagcctgtatttcaccatgctgaaccacaacaagcggtcgatcacgctcgacaccaagaacccgaagggcaaggaggtgctgaccgcgctgatccgcacctgcgacgtgctggtagagaatttcggccccggtgtgctcgaccggatgggcttcacctgggagaagatccaggagatcaacccgcggatgatcgtcgcctcgatcaagggcttcggtcccggcccgtatgaagactgcaaggtgtacgagaacgttgcgcagtgcaccggcggcgccgcctcgaccaccggattccgcgaaggcctgccgctggtcaccggcgcgcagatcggcgatagcggcaccggcctgcatctcgcgctcggcatcgtcaccgcgctgtatcagcgccaccacaccggccgcggccagcgcgtcaccgcggcgatgcaggacggcgtgctgaacctctgccgcgtcaagctgcgcgatcagcagcgcctcgaccatggtccgctgaaggaatacagccagttcggcgaaggcatcccgttcggcgatgcggtgccgcgtgccggcaacgattccggtggcggccagcccggccgcatcctgaagtgcaagggctgggagcaggatccgaacgcctacatctacgtcatcacccaggcgccggtgtgggagaagatctgcgacgtgatcggcgagaccggctggaagacgcaccccgactacgccacgccgccggcgcggctgtcgcggctcaacgagatcttcgcgcgcattgagcaatggaccatgaccaagaccaagttcgaggccatggagatcctcaacgccgacgacatcccctgcggcccgatcctgtcgatgaaggaactcgccgaagatcagtcgctgcgcgccaccggcaccatcgtcgaggtcgatcacccgacccgcggcaagtatctgtcggtcggcaacccgatcaagctgtcggactccccgaccgaggtgaagcgctcgccgctactcggtgaacacaccgacgaaatcctgcgcgacgtcctcggctacagcgacgcgcacgtcgcagagatccacgactccggcgcgaccgctccgccgcgcaagcaagcggcggagtaattgctgcaacgcattcgccgcgcccggacaccacgggcgcggccggactaactcgtcttgatgtaataacaagtcacgtcgttatttccctccttgcgcctggtgattagcacgatcacaatggcgacggtacggctcccgacagaggcgccggctcacggaggaaacatgacggacacggtgcacggggtggccgccgc

SEQ ID NO: 202 - DNA, 1221 bp, Artificial Sequence; identical to SEQ ID NO: 54
  Synthesized phaD

SEQ ID NO: 203 - DNA, 1212 bp, Artificial Sequence; identical to SEQ ID NO: 55
  Synthesized pcaF

SEQ ID NO: 204 - DNA, 1164 bp, Artificial Sequence; identical to SEQ ID NO: 56
  Synthesized fadA

SEQ ID NO: 205 - DNA, 1005 bp, Artificial Sequence; identical to SEQ ID NO: 57
  Snthesized atoB

SEQ ID NO: 206 - DNA, 1206 bp, Artificial Sequence; identical to SEQ ID NO: 58
  Synthesized pcaF

SEQ ID NO: 207 - DNA, 2373 bp, Artificial Sequence; identical to SEQ ID NO: 59
  Synthesized bktB

SEQ ID NO: 208 - DNA, 1209 bp, Artificial Sequence; identical to SEQ ID NO: 60
  Synthesized phaA

SEQ ID NO: 209 - DNA, 717 bp, Artificial Sequence; identical to SEQ ID NO: 61
  Synthesized Clo1313_1716

SEQ ID NO: 210 - DNA, 2846 bp, Artificial Sequence; identical to SEQ ID NO: 62
  Synthesized fadB

SEQ ID NO: 211 - DNA, 2789 bp, Artificial Sequence; identical to SEQ ID NO: 63
  Synthesized yfcX

SEQ ID NO: 212 - DNA, 952 bp, Artificial Sequence; identical to SEQ ID NO: 64
  Synthesized phbB

SEQ ID NO: 213 - DNA, 979 bp, Artificial Sequence; identical to SEQ ID NO: 65
  Synthesized phaB

SEQ ID NO: 214 - DNA, 810 bp, Artificial Sequence; identical to SEQ ID NO: 134
  Synthesized mhpD

SEQ ID NO: 215 - DNA, 789 bp, Artificial Sequence; identical to SEQ ID NO: 135
  Synthesized ctmF

SEQ ID NO: 216 - DNA, 804 bp, Artificial Sequence; identical to SEQ ID NO: 136
  Synthesized hpaH

SEQ ID NO: 217 - DNA, 507 bp, Artificial Sequence; identical to SEQ ID NO: 137
  Synthesized cnbE

SEQ ID NO: 218 - DNA, 1263 bp, Artificial Sequence; identical to SEQ ID NO: 138
  Synthesized dmdA

SEQ ID NO: 219 - DNA, 492 bp, Artificial Sequence; identical to SEQ ID NO: 139
  Synthesized dmdB

SEQ ID NO: 220 - DNA, 786 bp, Artificial Sequence; identical to SEQ ID NO: 140
  Synthesized crt

SEQ ID NO: 221 - DNA, 70 bp, Artificial Sequence; identical to SEQ ID NO: 49
  Synthesized lcdA

SEQ ID NO: 222 - DNA, 70 bp, Artificial Sequence; identical to SEQ ID NO: 51
  Synthesized lcdB

SEQ ID NO: 223 - DNA, 70 bp, Artificial Sequence; identical to SEQ ID NO: 52
  Synthesized lcdC

SEQ ID NO: 224 - DNA, 423 bp, Artificial Sequence; identical to SEQ ID NO: 66
  Synthesized Orfl

SEQ ID NO: 225 - DNA, 432 bp, Artificial Sequence; identical to SEQ ID NO: 67
  Synthesized COG0824

SEQ ID NO: 226 - DNA, 411 bp, Artificial Sequence; identical to SEQ ID NO: 68
  Synthesized Jann_0674

SEQ ID NO: 227 - DNA, 432 bp, Artificial Sequence; identical to SEQ ID NO: 69
  Synthesized SSE37_24444

SEQ ID NO: 228 - DNA, 414 bp, Artificial Sequence; identical to SEQ ID NO: 70
  Synthesized entH

SEQ ID NO: 229 - DNA, 651 bp, Artificial Sequence; identical to SEQ ID NO: 71
  Synthesized atoA

SEQ ID NO: 230 - DNA, 663 bp, Artificial Sequence; identical to SEQ ID NO: 72
  Synthesized atoD

SEQ ID NO: 231 - DNA, 1509 bp, Artificial Sequence; identical to SEQ ID NO: 73
  Synthesized actA

SEQ ID NO: 232 - DNA, 378 bp, Artificial Sequence; identical to SEQ ID NO: 74
  Synthesized Cg0592

SEQ ID NO: 233 - DNA, 657 bp, Artificial Sequence; identical to SEQ ID NO: 75
  Synthesized ctfA

SEQ ID NO: 234 - DNA, 666 bp, Artificial Sequence; identical to SEQ ID NO: 76
  Synthesized ctfB

SEQ ID NO: 235 - DNA, 1554 bp, Artificial Sequence; identical to SEQ ID NO: 77
  Synthesized Cbei_4543

SEQ ID NO: 236 - DNA, 654 bp, Artificial Sequence; identical to SEQ ID NO: 78
  Synthesized PcaJ

SEQ ID NO: 237 - DNA, 1269 bp, Artificial Sequence; identical to SEQ ID NO: 79
  Synthesized oleTJE

SEQ ID NO: 238 - DNA, 1555 bp, Artificial Sequence; identical to SEQ ID NO: 80
  Synthesized padA1

SEQ ID NO: 239 - DNA, 1680 bp, Artificial Sequence; identical to SEQ ID NO: 81
  Synthesized sdrA

SEQ ID NO: 240 - DNA, 1050 bp, Artificial Sequence; identical to SEQ ID NO: 82
  Synthesized mvd

SEQ ID NO: 241 - DNA, 1191 bp, Artificial Sequence; identical to SEQ ID NO: 83
  Synthesized mvd

SEQ ID NO: 242 - DNA, 1182 bp, Artificial Sequence; identical to SEQ ID NO: 84
  Synthesized mvd

SEQ ID NO: 243 - DNA, 972 bp, Artificial Sequence; identical to SEQ ID NO: 85
  Synthesized mvd

SEQ ID NO: 244 - DNA, 984 bp, Artificial Sequence; identical to SEQ ID NO: 86
  Synthesized mvd

SEQ ID NO: 245 - DNA, 951 bp, Artificial Sequence; identical to SEQ ID NO: 87
  Synthesized mvd

SEQ ID NO: 246 - DNA, 1005 bp, Artificial Sequence; identical to SEQ ID NO: 88
  Synthesized dvd

SEQ ID NO: 247 - DNA, 1300 bp, Artificial Sequence; identical to SEQ ID NO: 89
  Synthesized dfd

SEQ ID NO: 248 - DNA, 954 bp, Artificial Sequence; identical to SEQ ID NO: 90
  Synthesized mvaD

SEQ ID NO: 249 - DNA, 1005 bp, Artificial Sequence; identical to SEQ ID NO: 91
  Synthesized mvaD

SEQ ID NO: 250 - DNA, 939 bp, Artificial Sequence; identical to SEQ ID NO: 92
  Synthesized mvaD

SEQ ID NO: 251 - DNA, 2778 bp, Artificial Sequence; identical to SEQ ID NO: 93
  Synthesized pdc2

SEQ ID NO: 252 - DNA, 1707 bp, Artificial Sequence; identical to SEQ ID NO: 94
  Synthesized pdc1

(19) United States
(12) Patent Application Publication
(10) Pub. No.: US 2014/0134687 A1
(43) Pub. Date: May 15, 2014

(54) MODIFIED MICROORGANISMS AND METHODS OF USING SAME FOR PRODUCING BUTADIENE AND SUCCINATE

(71) Applicant: Braskem S/A AP 09, Sao Paulo - SP (BR)

(72) Inventors: Mateus Schreiner Garcez Lopes, Sao Paulo (BR); Avram Michael Slovic, Sao Paulo (BR)

(73) Assignee: Braskem S/A AP 09, Sao Paulo - SP (BR)

(21) Appl. No.: 14/044,545

(22) Filed: Oct. 2, 2013

Related U.S. Application Data
(60) Provisional application No. 61/708,895, filed on Oct. 2, 2012.

(51) Int. Cl. C12P 7/46; C12P 5/02

(52) U.S. Cl. CPC C12P 7/46; C12P 5/026
USPC 435/145; 435/252.31; 435/254.21; 435/254.23; 435/257.2; 435/254.11; 435/258.1; 435/252.33; 435/252.3

(57) ABSTRACT

The present disclosure generally relates to microorganisms (e.g., non-naturally occurring microorganisms) that comprise one or more polynucleotides coding for enzymes in a pathway that catalyze a conversion of a carbon source (e.g., a fermentable carbon source) to butadiene and succinate and the use of such microorganisms for the production of butadiene and succinate.

### BACKGROUND

Butadiene (1,3-butadiene, CH₂═CH—CH═CH₂, CAS 106-99-0) and succinate (butanedioic acid, HOOCCH₂CH₂COOH, CAS 110-15-6), are typically manufactured (along with other 4-carbon) in processes that involve harsh conditions and high temperatures 850° C.). Other methods for their production involve toxic and/or expensive catalysts, highly flammable and/or gaseous carbon sources, and high temperatures.

Butadiene and succinate are in demand globally and are used in a variety of commercial applications. For example, butadiene can be polymerized to form polybutadiene, or reacted with hydrogen cyanide (prussic acid) in the presence of a nickel catalyst to form adiponitrile, a precursor to nylon. More commonly, however, butadiene is polymerized with other olefins to form copolymers such as acrylonitrile-butadiene-styrene (ABS), acrylonitrile-butadiene (ABR), or styrene-butadiene (SBR) copolymers. Succinate is commonly used within the food and beverage industry, primarily as a sweetener.

Given the world-wide demand for butadiene and succinate derived therefrom, there exits a need in the art for improved methods for their production with other olefins and methods for their production which overcome their current production drawbacks including the use of toxic and/or expensive catalysts, and highly flammable and/or gaseous carbon sources.

### SUMMARY

The present disclosure generally relates to microorganisms (e.g., non-naturally occurring microorganisms) that comprise one or more polynucleotides coding for enzymes in a pathway that catalyze a conversion of a carbon source (e.g., a fermentable carbon source) to butadiene and succinate and the use of such microorganisms for the production of butadiene and succinate. The present disclosure also generally relates to microorganisms (e.g., non-naturally occurring microorganisms) that comprise one or more polynucleotides coding for enzymes in a pathway that catalyze a conversion of a carbon source (e.g., a fermentable carbon source) to butadiene and succinate, and the use of such microorganisms for the production of butadiene and succinate.

The present disclosure provides methods of co-producing butadiene and succinate from a fermentable carbon source, the method comprising: providing a fermentable carbon source; expressing one or more polynucleotides in a microorganism that encode one or more enzymes in a pathway that catalyze a conversion of the fermentable carbon source to one or more intermediates in a pathway for the production of butadiene and succinate, expressing one or more polynucleotides in the microorganism that encode one or more enzymes in a pathway that catalyze a conversion of the one or more intermediates into butadiene and succinate; and contacting the fermentable carbon source with the microorganism, wherein the one or more intermediates in the pathway for the production of butadiene are selected from the group consisting of: crotonyl-CoA, acryloyl-CoA, 3-hydroxypropionyl-CoA, and formyl-CoA, and wherein the co-production method is anaerobic.oxaloacetate

In some embodiments of each or any of the above or below mentioned embodiments, the enzymes that catalyze the conversion of the fermentable carbon source to one or more intermediates in the pathway for the production of butadiene and succinate are set forth in any one of FIGS. 1-4.

In some embodiments of each or any of the above or below mentioned embodiments, the enzymes that catalyze the conversion of the one or more intermediates to butadiene and succinate are set forth in any one of FIGS. 1-4.

In some embodiments of each or any of the above or below mentioned embodiments, the microorganism is a bacteria selected from the genera consisting of: Propionibacterium, Propionispira, Clostridium, Bacillus, Escherichia, Pelobacter, and Lactobacillus.

In some embodiments of each or any of the above or below mentioned embodiments, the microorganism is a eukaryote selected from the group consisting of yeast, filamentous fungi, protozoa, and algae.

In some embodiments of each or any of the above or below mentioned embodiments, the yeast is Saccharomyces cerevisiae or Pichia pastoris.

In some embodiments of each or any of the above or below mentioned embodiments, the fermentable carbon source comprises sugarcane juice, sugarcane molasses, hydrolyzed starch, hydrolyzed lignocellulosic materials, glucose, sucrose, fructose, lactate, lactose, xylose, pyruvate, or glycerol in any form or mixture thereof.

In some embodiments of each or any of the above or below mentioned embodiments, the fermentable carbon source is a monosaccharide, oligosaccharide, or polysaccharide.

In some embodiments of each or any of the above or below mentioned embodiments, the produced butadiene and succinate is secreted by the microorganism into the fermentation media.

In some embodiments of each or any of the above or below mentioned embodiments, the methods further comprise recovering the produced butadiene and succinate from the fermentation media.

In some embodiments of each or any of the above or below mentioned embodiments, the microorganism has been genetically modified to express the one or more polynucleotides coding for enzymes in a pathway that catalyze a conversion of the fermentable carbon source to one or more intermediates in a pathway for the production of butadiene and succinate, and one or more polynucleotides coding for enzymes in a pathway that catalyze a conversion of the one or more intermediates to butadiene and succinate.

The present disclosure also provides microorganisms that comprise one or more polynucleotides coding for enzymes in a pathway that catalyze a conversion of the fermentable carbon source to one or more intermediates in a pathway for the production of butadiene and succinate, and one or more polynucleotides coding for enzymes in a pathway that catalyze a conversion of the one or more intermediates to butadiene and succinate, wherein the one or more intermediates in the pathway for the production of butadiene are selected from the group consisting of: crotonyl-CoA, acryloyl-CoA, 3-hydroxypropionyl-CoA, and formyl-CoA.

In some embodiments of each or any of the above or below mentioned embodiments, the enzymes that catalyze a conversion of the fermentable carbon source to one or more intermediates in the pathway for the production of butadiene and succinate are set forth in any one of FIGS. 1-4.

In some embodiments of each or any of the above or below mentioned embodiments, the enzymes that catalyze a conversion of the one or more intermediates to butadiene and succinate are set forth in any one of FIGS. 1-4.

In some embodiments of each or any of the above or below mentioned embodiments, the microorganism is a bacteria selected from the genera consisting of: Propionibacterium, Propionispira, Clostridium, Bacillus, Escherichia, Pelobacter, and Lactobacillus.

In some embodiments of each or any of the above or below mentioned embodiments, the microorganism is a eukaryote selected from the group consisting of yeast, filamentous fungi, protozoa, and algae.

In some embodiments of each or any of the above or below mentioned embodiments, the yeast is Saccharomyces cerevisiae or Pichia pastoris.

In some embodiments of each or any of the above or below mentioned embodiments, the microorganism has been genetically modified to express the one or more polynucleotides coding for enzymes in a pathway that catalyze a conversion of the fermentable carbon source to one or more intermediates in a pathway for the production of butadiene and succinate, and one or more polynucleotides coding for enzymes in a pathway that catalyze a conversion of the one or more intermediates to butadiene and succinate.

The present disclosure also provides methods of producing butadiene and succinate from a fermentable carbon source, comprising: providing a fermentable carbon source; contacting the fermentable carbon source with a microorganism comprising one or more polynucleotides coding for enzymes in a pathway that catalyze a conversion of the fermentable carbon source to one or more intermediates in a pathway for the production of butadiene and succinate, and one or more polynucleotides coding for enzymes in a pathway that catalyze a conversion of the one or more intermediates to butadiene and succinate, adipate, and/or butanol in a fermentation media; and expressing the one or more polynucleotides coding for the enzymes in the pathway that catalyze a conversion of the fermentable carbon source to one or more intermediates in a pathway for the production of butadiene and succinate and one or more polynucleotides coding for the enzymes in the pathway that catalyze a conversion of the one or more intermediates to butadiene and succinate, adipate, and/or butanol in the microorganism to produce butadiene and succinate.

In some embodiments of each or any of the above or below mentioned embodiments, the enzymes that catalyze the conversion of the fermentable carbon source to one or more intermediates in the pathway for the production of butadiene and succinate, are set forth in any one of FIGS. 1-4.

In some embodiments of each or any of the above or below mentioned embodiments, the enzymes that catalyze the conversion of the one or more intermediates to butadiene and succinate are set forth in any one of FIGS. 1-4.

In some embodiments of each or any of the above or below mentioned embodiments, butadiene and succinate are produced, butadiene and adipate are produced, or butadiene and butanol are produced.

In some embodiments of each or any of the above or below mentioned embodiments, butadiene is produced via a crotonyl-CoA, acryloyl-CoA, 3-hydroxypropionyl-CoA and formyl-CoA intermediate.

In some embodiments of each or any of the above or below mentioned embodiments, succinate, adipate, and/or butanol are produced via an oxalacetate intermediate.

In some embodiments of each or any of the above or below mentioned embodiments, the microorganism is a bacteria selected from the genera consisting of: Propionibacterium, Propionispira, Clostridium, Bacillus, Escherichia, Pelobacter, and Lactobacillus.

In some embodiments of each or any of the above or below mentioned embodiments, the microorganism is a eukaryote selected from the group consisting of yeast, filamentous fungi, protozoa, and algae.

In some embodiments of each or any of the above or below mentioned embodiments, the yeast is Saccharomyces cerevisiae or Pichia pastoris.

In some embodiments of each or any of the above or below mentioned embodiments, the fermentable carbon source comprises sugarcane juice, sugarcane molasses, hydrolyzed starch, hydrolyzed lignocellulosic materials, glucose, sucrose, fructose, lactate, lactose, xylose, pyruvate, or glycerol in any form or mixture thereof.

In some embodiments of each or any of the above or below mentioned embodiments, the fermentable carbon source is a monosaccharide, oligosaccharide, or polysaccharide.

In some embodiments of each or any of the above or below mentioned embodiments, the produced butadiene, and succinate, adipate, and/or butanol are secreted by the microorganism into the fermentation media.

In some embodiments of each or any of the above or below mentioned embodiments the methods further comprise recovering the produced butadiene, and succinate, adipate, and/or butanol from the fermentation media.

In some embodiments of each or any of the above or below mentioned embodiments, the microorganism has been genetically modified to express the one or more polynucleotides coding for enzymes in a pathway that catalyze a conversion of the fermentable carbon source to one or more intermediates in a pathway for the production of butadiene and succinate, and one or more polynucleotides coding for enzymes in a pathway that catalyze a conversion of the one or more intermediates to butadiene and succinate.

In some embodiments of each or any of the above or below mentioned embodiments, the conversion of the fermentable carbon source to butadiene and succinate is anaerobic.

The present disclosure also provides a microorganism comprising one or more polynucleotides coding for enzymes in a pathway that catalyze a conversion of the fermentable carbon source to one or more intermediates in a pathway for the production of butadiene and succinate, and one or more polynucleotides coding for enzymes in a pathway that catalyze a conversion of the one or more intermediates to butadiene and succinate.

In some embodiments of each or any of the above or below mentioned embodiments, the enzymes that catalyze a conversion of the fermentable carbon source to one or more intermediates in the pathway for the production of butadiene and succinate are set forth in any one of FIGS. 1-4.

In some embodiments of each or any of the above or below mentioned embodiments, the enzymes that catalyze a conversion of the one or more intermediates to butadiene and succinate, adipate, and/or butanol are set forth in any one of FIGS. 1-4.

In some embodiments of each or any of the above or below mentioned embodiments, butadiene is produced via a crotonyl-CoA, acryloyl-CoA, 3-hydroxypropionyl-CoA, and formyl-CoA intermediate.

In some embodiments of each or any of the above or below mentioned embodiments, succinate is produced via an oxalacetate intermediate.

In some embodiments of each or any of the above or below mentioned embodiments, the microorganism is a bacteria selected from the genera consisting of: Propionibacterium, Propionispira, Clostridium, Bacillus, Escherichia, Pelobacter, and Lactobacillus.

In some embodiments of each or any of the above or below mentioned embodiments, the microorganism is a eukaryote selected from the group consisting of yeast, filamentous fungi, protozoa, and algae.

In some embodiments of each or any of the above or below mentioned embodiments, the yeast is Saccharomyces cerevisiae or Pichia pastoris.

In some embodiments of each or any of the above or below mentioned embodiments, the microorganism has been genetically modified to express the one or more polynucleotides coding for enzymes in a pathway that catalyze a conversion of the fermentable carbon source to one or more intermediates in a pathway for the production of butadiene and succinate, and one or more polynucleotides coding for enzymes in a pathway that catalyze a conversion of the one or more intermediates to butadiene and succinate.

### BRIEF DESCRIPTION OF THE DRAWINGS

The foregoing summary, as well as the following detailed description of the disclosure, will be better understood when read in conjunction with the appended figures. For the purpose of illustrating the disclosure, shown in the figures are embodiments which are presently preferred. It should be understood, however, that the disclosure is not limited to the precise arrangements, examples and instrumentalities shown.

FIG. 1 depicts an exemplary pathway for the production of succinate via an oxalacetate intermediate and butadiene via a crotonyl-CoA intermediate.

FIG. 2 depicts an exemplary pathway for the production of succinate via an oxalacetate intermediate and butadiene via an acryloyl-CoA intermediate.

FIG. 3 depicts an exemplary pathway for the production of succinate via an oxalacetate intermediate and butadiene via a 3-hydroxypropionyl-CoA intermediate.

FIG. 4 depicts an exemplary pathway for the production of succinate via an oxalacetate intermediate and butadiene via a formyl-CoA intermediate.

### DETAILED DESCRIPTION

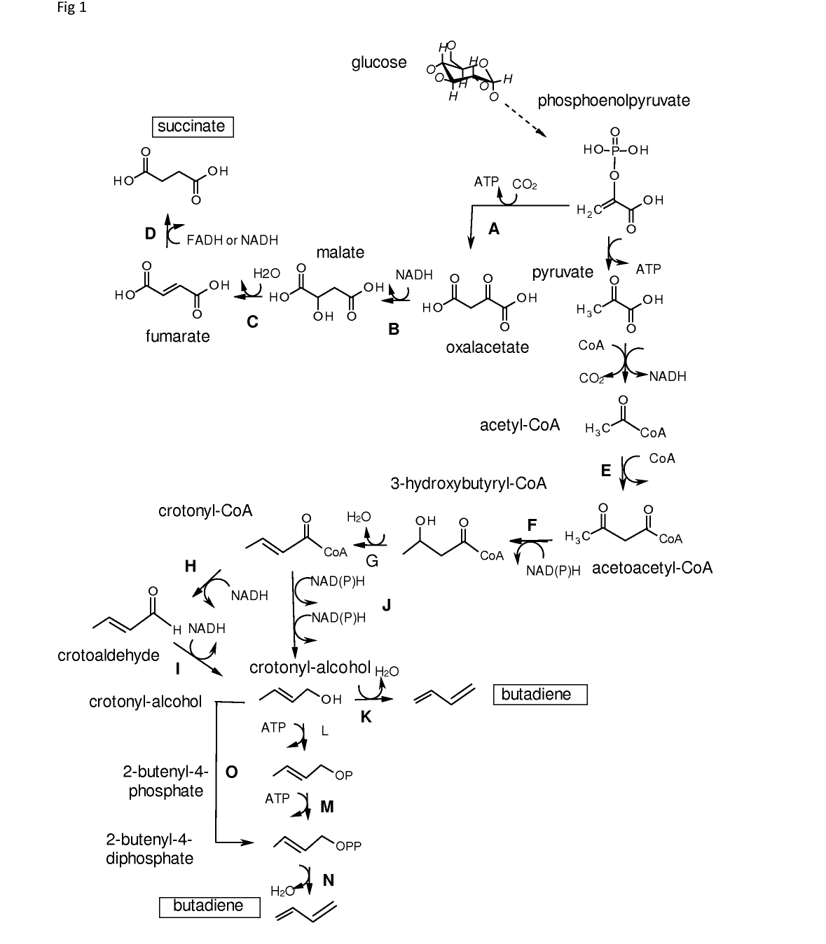

The present disclosure generally relates to microorganisms (e.g., non-naturally occurring microorganisms) that comprise one or more genetically modified pathways and uses of such microorganisms for the conversion of a fermentable carbon source to butadiene and succinate (see, FIGS. 1-4). Such microorganisms may comprise one or more polynucleotides coding for enzymes that catalyze a conversion of a fermentable carbon source to butadiene and succinate. The present disclosure generally relates to microorganisms (e.g., non-naturally occurring microorganisms) that comprise one or more genetically modified pathways and uses of such microorganisms for the conversion of a fermentable carbon source to butadiene and succinate (see, FIGS. 1-4). Such microorganisms may comprise one or more polynucleotides coding for enzymes that catalyze a conversion of a fermentable carbon source to butadiene and succinate.

This disclosure provides, in part, the discovery of novel enzymatic pathways including, for example, novel combinations of enzymatic pathways, for the production of butadiene and succinate from a carbon source (e.g., a fermentable carbon source). In an embodiment, butadiene and succinate are produced.

The methods provided herein provide end-results similar to those of sterilization without the high capital expenditure and continuing higher management costs that are typically required to establish and maintain sterility throughout a production process. In this regard, most industrial-scale butadiene and succinate production processes are operated in the presence of measurable numbers of bacterial contaminants due to the aerobic nature of their processes. It is believed that bacterial contamination of an butadiene and succinate, adipate, and/or butanol production processes causes a reduction in product yield and an inhibition of growth of the microorganism producing butadiene and succinate. Such drawbacks of prior methods are avoided by the presently disclosed methods as the toxic nature of the produced butadiene and succinate, adipate, and/or butanol reduces contaminants in the production process.

The enzymatic pathways disclosed herein are advantageous over prior known enzymatic pathways for the production of butadiene and succinate in that the enzymatic pathways disclosed herein are anaerobic. While it is possible to use aerobic processes to produce butadiene and succinate, anaerobic processes are preferred due to the risk incurred when olefins (which are by nature are explosive) are mixed with oxygen during the fermentation process. Moreover, the supplementation of oxygen and nitrogen in a fermenter requires an additional investment for aerobic process and another additional investment for the purification from the nitrogen from the butadiene and succinate. The presence of oxygen can also catalyze the polymerization of butadiene and can promote the growth of aerobic contaminants in the fermentor broth. Additionally, aerobic fermentation processes for the production of butadiene present several drawbacks at industrial scale (where it is technically challenging to maintain aseptic conditions) such as the fact that: (i) greater biomass is obtained reducing overall yields on carbon for the desired products; (ii) the presence and oxygen favors the growth of contaminants (Weusthuis et al., 2011, Trends in Biotechnology, 2011, Vol. 29, No. 4, 153-158) and (iii) the mixture of oxygen and gaseous compounds such as butadiene poses serious risks of explosion, (iv) the oxygen can catalyze the unwanted reaction of polymerization of the olefin and, finally, (v) higher costs of fermentation and purification in aerobic conditions. Each of the drawbacks associated with aerobic fermentation including, for example, the risk of an explosion during the manufacture of butadiene and dilution by oxygen and nitrogen are overcome by the anaerobic fermentation methods provided herein.

It will be understood that the steps involved in any and all of the methods described herein may be performed in any order and are not to be limited or restricted to the order in which they are particularly recited. For example, the present disclosure provides methods of co-producing butadiene and succinate from a fermentable carbon source, comprising: providing a fermentable carbon source; expressing one or more polynucleotides in a microorganism that encode one or more enzymes in a pathway that catalyze a conversion of the fermentable carbon source to one or more intermediates in a pathway for the production of butadiene and succinate, expressing one or more polynucleotides in the microorganism that encode one or more enzymes in a pathway that catalyze a conversion of the one or more intermediates into butadiene and succinate, and contacting the fermentable carbon source with the microorganism. As such, expressing one or more polynucleotides in a microorganism that encode one or more enzymes in a pathway that catalyze a conversion of the fermentable carbon source to one or more intermediates in a pathway for the production of butadiene and succinate and/or expressing one or more polynucleotides in the microorganism that encode one or more enzymes in a pathway that catalyze a conversion of the one or more intermediates into butadiene and succinate may be performed prior to or after contacting the fermentable carbon source with the microorganism.

As used herein, the term “biological activity” or “functional activity,” when referring to a protein, polypeptide or peptide, may mean that the protein, polypeptide or peptide exhibits a functionality or property that is useful as relating to some biological process, pathway or reaction. Biological or functional activity can refer to, for example, an ability to interact or associate with (e.g., bind to) another polypeptide or molecule, or it can refer to an ability to catalyze or regulate the interaction of other proteins or molecules (e.g., enzymatic reactions).

As used herein, “butadiene” is intended to mean 1,3-butadiene with a general formula CH₂═CH═CH═CH₂ (CAS number-106-99-0).

As used herein, the term “culturing” may refer to growing a population of cells, e.g., microbial cells, under suitable conditions for growth, in a liquid or on solid medium.

As used herein, the term “derived from” may encompass the terms originated from, obtained from, obtainable from, isolated from, and created from, and generally indicates that one specified material finds its origin in another specified material or has features that can be described with reference to the another specified material.

As used herein, “exogenous polynucleotide” refers to any deoxyribonucleic acid that originates outside of the microorganism.

As used herein, the term “an expression vector” may refer to a DNA construct containing a polynucleotide or nucleic acid sequence encoding a polypeptide or protein, such as a DNA coding sequence (e.g. gene sequence) that is operably linked to one or more suitable control sequence(s) capable of affecting expression of the coding sequence in a host. Such control sequences include a promoter to affect transcription, an optional operator sequence to control such transcription, a sequence encoding suitable mRNA ribosome binding sites, and sequences which control termination of transcription and translation. The vector may be a plasmid, cosmid, phage particle, bacterial artificial chromosome, or simply a potential genomic insert. Once transformed into a suitable host, the vector may replicate and function independently of the host genome (e.g., independent vector or plasmid), or may, in some instances, integrate into the genome itself (e.g., integrated vector). The plasmid is the most commonly used form of expression vector. However, the disclosure is intended to include such other forms of expression vectors that serve equivalent functions and which are, or become, known in the art.

As used herein, the term “expression” may refer to the process by which a polypeptide is produced based on a nucleic acid sequence encoding the polypeptides (e.g., a gene). The process includes both transcription and translation.

As used herein, the term “gene” may refer to a DNA segment that is involved in producing a polypeptide or protein (e.g., fusion protein) and includes regions preceding and following the coding regions as well as intervening sequences (introns) between individual coding segments (exons).

As used herein, the term “heterologous,” with reference to a nucleic acid, polynucleotide, protein or peptide, may refer to a nucleic acid, polynucleotide, protein or peptide that does not naturally occur in a specified cell, e.g., a host cell. It is intended that the term encompass proteins that are encoded by naturally occurring genes, mutated genes, and/or synthetic genes. In contrast, the term homologous, with reference to a nucleic acid, polynucleotide, protein or peptide, refers to a nucleic acid, polynucleotide, protein or peptide that occurs naturally in the cell.

As used herein, the term a “host cell” may refer to a cell or cell line, including a cell such as a microorganism which a recombinant expression vector may be transfected for expression of a polypeptide or protein (e.g., fusion protein). Host cells include progeny of a single host cell, and the progeny may not necessarily be completely identical (in morphology or in total genomic DNA complement) to the original parent cell due to natural, accidental, or deliberate mutation. A host cell may include cells transfected or transformed in vivo with an expression vector.

As used herein, the term “introduced,” in the context of inserting a nucleic acid sequence or a polynucleotide sequence into a cell, may include transfection, transformation, or transduction and refers to the incorporation of a nucleic acid sequence or polynucleotide sequence into a eukaryotic or prokaryotic cell wherein the nucleic acid sequence or polynucleotide sequence may be incorporated into the genome of the cell (e.g., chromosome, plasmid, plastid, or mitochondrial DNA), converted into an autonomous replicon, or transiently expressed.

As used herein, the term “non-naturally occurring” when used in reference to a microbial organism or microorganism of the invention is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. A non-naturally occurring microbial organisms of the disclosure can contain stable genetic alterations, which refers to microorganisms that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefinitely. Those skilled in the art will understand that the genetic alterations, including metabolic modifications exemplified herein, are described with reference to a suitable host organism such as E. coli and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the E. coli metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

As used herein, the term “operably linked” may refer to a juxtaposition or arrangement of specified elements that allows them to perform in concert to bring about an effect. For example, a promoter may be operably linked to a coding sequence if it controls the transcription of the coding sequence.

As used herein, the term “a promoter” may refer to a regulatory sequence that is involved in binding RNA polymerase to initiate transcription of a gene. A promoter may be an inducible promoter or a constitutive promoter. An inducible promoter is a promoter that is active under environmental or developmental regulatory conditions.

As used herein, the term “a polynucleotide” or “nucleic acid sequence” may refer to a polymeric form of nucleotides of any length and any three-dimensional structure and single- or multi-stranded (e.g., single-stranded, double-stranded, triple-helical, etc.), which contain deoxyribonucleotides, ribonucleotides, and/or analogs or modified forms of deoxyribonucleotides or ribonucleotides, including modified nucleotides or bases or their analogs. Such polynucleotides or nucleic acid sequences may encode amino acids (e.g., polypeptides or proteins such as fusion proteins). Because the genetic code is degenerate, more than one codon may be used to encode a particular amino acid, and the present disclosure encompasses polynucleotides which encode a particular amino acid sequence. Any type of modified nucleotide or nucleotide analog may be used, so long as the polynucleotide retains the desired functionality under conditions of use, including modifications that increase nuclease resistance (e.g., deoxy, 2′-O-Me, phosphorothioates, etc.). Labels may also be incorporated for purposes of detection or capture, for example, radioactive or nonradioactive labels or anchors, e.g., biotin. The term polynucleotide also includes peptide nucleic acids (PNA). Polynucleotides may be naturally occurring or non-naturally occurring. The terms polynucleotide, nucleic acid, and oligonucleotide are used herein interchangeably. Polynucleotides may contain RNA, DNA, or both, and/or modified forms and/or analogs thereof. A sequence of nucleotides may be interrupted by non-nucleotide components. One or more phosphodiester linkages may be replaced by alternative linking groups. These alternative linking groups include, but are not limited to, embodiments wherein phosphate is replaced by P(O)S (thioate), P(S)S (dithioate), (O)NR₂ (amidate), P(O)R, P(O)OR′, COCH₂ (formacetal), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. Polynucleotides may be linear or circular or comprise a combination of linear and circular portions.

As used herein, the term a “protein” or “polypeptide” may refer to a composition comprised of amino acids and recognized as a protein by those of skill in the art. The conventional one-letter or three-letter code for amino acid residues is used herein. The terms protein and polypeptide are used interchangeably herein to refer to polymers of amino acids of any length, including those comprising linked (e.g., fused) peptides/polypeptides (e.g., fusion proteins). The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, related proteins, polypeptides or peptides may encompass variant proteins, polypeptides or peptides. Variant proteins, polypeptides or peptides differ from a parent protein, polypeptide or peptide and/or from one another by a small number of amino acid residues. In some embodiments, the number of different amino acid residues is any of about 1, 2, 3, 4, 5, 10, 20, 25, 30, 35, 40, 45, or 50. In some embodiments, variants differ by about 1 to about 10 amino acids. Alternatively or additionally, variants may have a specified degree of sequence identity with a reference protein or nucleic acid, e.g., as determined using a sequence alignment tool, such as BLAST, ALIGN, and CLUSTAL (see, infra). For example, variant proteins or nucleic acid may have at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or even 99.5% amino acid sequence identity with a reference sequence.

As used herein, the term “recovered,” “isolated,” “purified,” and “separated” may refer to a material (e.g., a protein, peptide, nucleic acid, polynucleotide or cell) that is removed from at least one component with which it is naturally associated. For example, these terms may refer to a material which is substantially or essentially free from components which normally accompany it as found in its native state, such as, for example, an intact biological system.

As used herein, the term “recombinant” may refer to nucleic acid sequences or polynucleotides, polypeptides or proteins, and cells based thereon, that have been manipulated by man such that they are not the same as nucleic acids, polypeptides, and cells as found in nature. Recombinant may also refer to genetic material (e.g., nucleic acid sequences or polynucleotides, the polypeptides or proteins they encode, and vectors and cells comprising such nucleic acid sequences or polynucleotides) that has been modified to alter its sequence or expression characteristics, such as by mutating the coding sequence to produce an altered polypeptide, fusing the coding sequence to that of another coding sequence or gene, placing a gene under the control of a different promoter, expressing a gene in a heterologous organism, expressing a gene at decreased or elevated levels, expressing a gene conditionally or constitutively in manners different from its natural expression profile, and the like.

As used herein, the term “selective marker” or “selectable marker” may refer to a gene capable of expression in a host cell that allows for ease of selection of those hosts containing an introduced nucleic acid sequence, polynucleotide or vector. Examples of selectable markers include but are not limited to antimicrobial substances (e.g., hygromycin, bleomycin, or chloramphenicol) and/or genes that confer a metabolic advantage, such as a nutritional advantage, on the host cell.

As used herein, the term “substantially similar” and “substantially identical” in the context of at least two nucleic acids, polynucleotides, proteins or polypeptides may mean that a nucleic acid, polynucleotide, protein or polypeptide comprises a sequence that has at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or even 99.5% sequence identity, in comparison with a reference (e.g., wild-type) nucleic acid, polynucleotide, protein or polypeptide. Sequence identity may be determined using known programs such as BLAST, ALIGN, and CLUSTAL using standard parameters. (See, e.g., Altshul et al. (1990) J. Mol. Biol. 215:403-410; Henikoff et al. (1989) Proc. Natl. Acad. Sci. 89:10915; Karin et al. (1993) Proc. Natl. Acad. Sci. 90:5873; and Higgins et al. (1988) Gene 73:237). Software for performing BLAST analyses is publicly available through the National Center for Biotechnology Information. Also, databases may be searched using FASTA (Person et al. (1988) Proc. Natl. Acad. Sci. 85:2444-2448.) In some embodiments, substantially identical polypeptides differ only by one or more conservative amino acid substitutions. In some embodiments, substantially identical polypeptides are immunologically cross-reactive. In some embodiments, substantially identical nucleic acid molecules hybridize to each other under stringent conditions (e.g., within a range of medium to high stringency).

As used herein, “succinate” or “succinic acid” is intended to mean a dicarboxylic add with a general molecular formula of C4H6O4 (CAS number-110-15-6).

As used herein, the term “transfection” or “transformation” may refer to the insertion of an exogenous nucleic acid or polynucleotide into a host cell. The exogenous nucleic acid or polynucleotide may be maintained as a non-integrated vector, for example, a plasmid, or alternatively, may be integrated into the host cell genome. The term transfecting or transfection is intended to encompass all conventional techniques for introducing nucleic acid or polynucleotide into host cells. Examples of transfection techniques include, but are not limited to, calcium phosphate precipitation, DEAE-dextran-mediated transfection, lipofection, electroporation, and microinjection.

As used herein, the term “transformed,” “stably transformed,” and “transgenic” may refer to a cell that has a non-native (e.g., heterologous) nucleic acid sequence or polynucleotide sequence integrated into its genome or as an episomal plasmid that is maintained through multiple generations.

As used herein, the term “vector” may refer to a polynucleotide sequence designed to introduce nucleic acids into one or more cell types. Vectors include cloning vectors, expression vectors, shuttle vectors, plasmids, phage particles, single and double stranded cassettes and the like.

As used herein, the term “wild-type,” “native,” or “naturally-occurring” proteins may refer to those proteins found in nature. The terms wild-type sequence refers to an amino acid or nucleic acid sequence that is found in nature or naturally occurring. In some embodiments, a wild-type sequence is the starting point of a protein engineering project, for example, production of variant proteins.

Unless defined otherwise herein, all technical and scientific terms used herein have the same meaning as commonly understood by one of ordinary skill in the art to which this disclosure belongs. Singleton, et al., Dictionary of Microbiology and Molecular Biology, second ed., John Wiley and Sons, New York (1994), and Hale & Markham, The Harper Collins Dictionary of Biology, Harper Perennial, NY (1991) provide one of skill with a general dictionary of many of the terms used in this disclosure.

Numeric ranges provided herein are inclusive of the numbers defining the range.

Unless otherwise indicated, nucleic acids sequences are written left to right in 5′ to 3′ orientation; amino acid sequences are written left to right in amino to carboxy orientation, respectively.

While the present disclosure is capable of being embodied in various forms, the description below of several embodiments is made with the understanding that the present disclosure is to be considered as an exemplification of the disclosure, and is not intended to limit the disclosure to the specific embodiments illustrated. Headings are provided for convenience only and are not to be construed to limit the disclosure in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” Also, the disclosure of ranges is intended as a continuous range including every value between the minimum and maximum values recited as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present disclosure.

### Modification of Microorganism

A microorganism may be modified (e.g., genetically engineered) by any method known in the art to comprise and/or express one or more polynucleotides coding for enzymes in a pathway that catalyze a conversion of a fermentable carbon source to one or more intermediates in a pathway for the co-production of butadiene with succinate, adipate, and/or butanol (e.g., butadiene and succinate; butadiene and adipate; and butadiene and butanol). Such enzymes may include any of those enzymes as set forth in any one of FIGS. 1-15.

For example, the microorganism may be modified to comprise one or more polynucleotides coding for enzymes that catalyze a conversion of crotonyl-CoA, acryloyl-CoA, 3-hydroxypropionyl-CoA, formyl-CoA, and/or malonyl-CoA to butadiene and one or more enzymes that catalyze a conversion of another intermediate such as oxalacetate to succinate.

In some embodiments, the one or more polynucleotides coding for enzymes in a pathway that catalyze a conversion of oxalacetate to succinate, and a conversion of crotonyl-CoA to butadiene include:
one or more polynucleotides coding for enzymes in a pathway that catalyze a conversion of PEP to oxaloacetate (e.g., PEP carboxykinase or PEP carboxylase);
one or more polynucleotides coding for enzymes in a pathway that catalyze a conversion of pyruvate to malate (e.g., pyruvate carboxylase);
one or more polynucleotides coding for enzymes in a pathway that catalyze a conversion of oxaloacetate to malate (e.g., malate dehydrogenase);
one or more polynucleotides coding for enzymes in a pathway that catalyze a conversion of malate to fumarate (e.g., fumarase);
one or more polynucleotides coding for enzymes in a pathway that catalyze a conversion of fumarate to succinate (e.g., fumarate reductase);
one or more polynucleotides coding for enzymes in a pathway that catalyze a conversion of 2 acetyl-CoA to acetoacetyl-CoA (e.g., acetyl-CoA thiolase);
one or more polynucleotides coding for enzymes in a pathway that catalyze a conversion of acetoacetyl-CoA to 3-hydroxybutyryl-CoA (e.g., 3-hydroxybutyryl-CoA dehydrogenase);
one or more polynucleotides coding for enzymes in a pathway that catalyze a conversion of 3-hydroxybutyryl-CoA to crotonyl-CoA (e.g., crotonase);
one or more polynucleotides coding for enzymes in a pathway that catalyze a conversion of crotonyl-CoA to crotoaldehyde (e.g., crotonaldehyde dehydrogenase);
one or more polynucleotides coding for enzymes in a pathway that catalyze a conversion of crotoaldehyde to crotonyl alcohol (e.g., alcohol dehydrogenase);
one or more polynucleotides coding for enzymes in a pathway that catalyze a conversion of crotonyl-CoA to crotonyl alcohol (e.g., crotonyl-CoA reductase (bifunctional));
one or more polynucleotides coding for enzymes in a pathway that catalyze a conversion of crotonyl alcohol to butadiene (e.g., crotonyl alcohol dehydratase);
one or more polynucleotides coding for enzymes in a pathway that catalyze a conversion of crotonyl alcohol to 2-butenyl-4-phosphate (e.g., crotonyl alcohol kinase);
one or more polynucleotides coding for enzymes in a pathway that catalyze a conversion of 2-butenyl-4-phosphate to 2-butenyl-4-diphosphate (e.g., 2-butenyl-4-phosphate kinase);
one or more polynucleotides coding for enzymes in a pathway that catalyze a conversion of 2-butenyl-4-diphosphate to butadiene (e.g., butadiene synthase);
one or more polynucleotides coding for enzymes in a pathway that catalyze a conversion of crotonyl-alcohol to 2-butenyl-4-diphosphate (e.g., crotonyl alcohol diphosphokinase);

Exemplary enzymes that convert oxalacetate to succinate and crotonyl-CoA to butadiene including, enzyme substrates, and enzyme reaction products associated with the conversions are presented in Table 1 below. The enzyme reference identifier listed in Table 1 correlates with the enzyme numbering used in FIG. 1, which schematically represents the enzymatic conversion of a fermentable carbon source to succinate and butadiene.

TABLE 1
Production of succinate via an oxalacetate intermediate and
butadiene via a crotonyl-CoA intermediate.

| Enzyme No. | Enzyme Name | Mediated Conversion |
|---|---|---|
| A. | PEP carboxykinase or | PEP → oxaloacetate |
| | PEP carboxylase or | PEP → oxaloacetate |
| | pyruvate carboxylase | pyruvate → malate |
| B. | malate dehydrogenase | oxaloacetate → malate |
| C. | fumarase | malate → fumarate |
| D. | fumarate reductase | fumarate → succinate |
| E. | acetyl-CoA thiolase | 2 acetyl-CoA → acetoacetyl-CoA |
| F. | 3-hydroxybutyryl-CoA | acetoacetyl-CoA → 3-hydroxybutyryl-CoA |
| | dehydrogenase | |
| G. | crotonase | 3-hydroxybutyryl-CoA → crotonyl-CoA |
| H. | crotonaldehyde dehydrogenase | crotonyl-CoA→ crotoaldehyde |
| I. | Crotonyl alcohol dehydrogenase | crotoaldehyde → crotonyl alcohol |
| J. | crotonyl-CoA reductase | crotonyl-CoA → crotonyl alcohol |
| | (bifunctional) | |
| K. | crotonyl alcohol dehydratase | crotonyl alcohol → butadiene |
| L. | crotonyl alcohol kinase | crotonyl alcohol → 2-butenyl-4-phosphate |
| M. | 2-butenyl-4-phosphate kinase | 2-butenyl-4-phosphate →2-butenyl-4- |
| | | diphosphate |
| N. | butadiene synthase | 2-butenyl-4-diphosphate → butadiene |
| O. | crotonyl alcohol diphosphokinase | crotonyl-alcohol → 2-butenyl-4-diphosphate |

In some embodiments, the one or more polynucleotides coding for enzymes in a pathway that catalyze a conversion of oxalacetate to succinate, and a conversion of acryloyl-CoA to butadiene include:
one or more polynucleotides coding for enzymes in a pathway that catalyze a conversion of PEP to oxaloacetate (e.g., PEP carboxykinase or PEP carboxylase);
one or more polynucleotides coding for enzymes in a pathway that catalyze a conversion of pyruvate to malate (e.g., pyruvate carboxylase);
one or more polynucleotides coding for enzymes in a pathway that catalyze a conversion of oxaloacetate to malate (e.g., malate dehydrogenase);
one or more polynucleotides coding for enzymes in a pathway that catalyze a conversion of malate to fumarate (e.g., fumarase);
one or more polynucleotides coding for enzymes in a pathway that catalyze a conversion of fumarate to succinate (e.g., fumarate reductase);
one or more polynucleotides coding for enzymes in a pathway that catalyze a conversion of pyruvate to R/S lactate (e.g., lactate dehydrogenase);
one or more polynucleotides coding for enzymes in a pathway that catalyze a conversion of R/S lactate to lactoyl-CoA (e.g., lactoyl-CoA transferase or synthase);
one or more polynucleotides coding for enzymes in a pathway that catalyze a conversion of lactoyl-CoA to acryloyl-CoA (e.g., lactoyl-CoA dehydratase);
one or more polynucleotides coding for enzymes in a pathway that catalyze a conversion of acryloyl-CoA and acetyl-CoA to 3-keto-pent-4-enoyl-CoA (e.g., keto-4-pentenoyl-CoA thiolase);
one or more polynucleotides coding for enzymes in a pathway that catalyze a conversion of 3-keto-pent-4-enoyl-CoA to R/S 3-hydroxy-4-pentenoyl-CoA (e.g., 3-keto-4-pentenoyl-CoA dehydrogenase);
one or more polynucleotides coding for enzymes in a pathway that catalyze a conversion of R/S 3-hydroxy-4-pentenoyl-CoA to 3-hydroxypent-4-enoic acid (e.g., 3-hydroxy-4-pentenoyl-CoA transferase or hydrolase or synthase); and/or
one or more polynucleotides coding for enzymes in a pathway that catalyze a conversion of 3-hydroxypent-4-enoic acid to butadiene (e.g., 3-hydroxy-4-pentenoic acid decarboxylase.

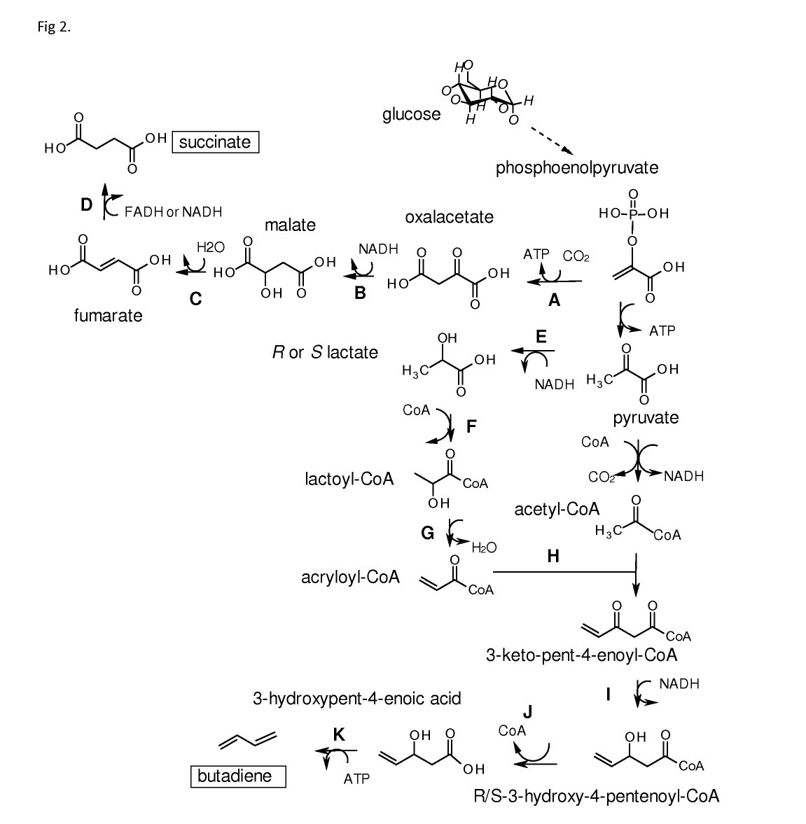

Exemplary enzymes that convert oxalacetate to succinate and acryloyl-CoA to butadiene including, enzyme substrates, and enzyme reaction products associated with the conversions are presented in Table 2 below. The enzyme reference identifier listed in Table 2 correlates with the enzyme numbering used in FIG. 2, which schematically represents the enzymatic conversion of a fermentable carbon source to succinate and butadiene.

TABLE 2
Production of succinate via an oxalacetate intermediate and butadiene via an
acryloyl-CoA intermediate.

| Enzyme No. | Enzyme Name | Mediated Conversion |
|---|---|---|
| A. | PEP carboxykinase or | PEP → oxaloacetate |
| | PEP carboxylase or | PEP → oxaloacetate |
| | Pyruvate Carboxylase | pyruvate → malate |
| B. | malate dehydrogenase | oxaloacetate → malate |
| C. | fumarase | malate → fumarate |
| D. | fumarate reductase | fumarate → succinate |
| E. | lactate dehydrogenase | pyruvate → R/S lactate |
| F. | lactoyl-CoA transferase | R/S lactate → lactoyl-CoA |
| | or synthase | |
| G. | lactoyl-CoA dehydratase | lactoyl-CoA → acryloyl-CoA |
| H. | keto-4-pentenoyl-CoA thiolase | acryloyl-CoA + acetyl-CoA → 3-keto-pent-4- |
| | | enoyl-CoA |
| I. | 3-keto-4-pentenoyl-CoA | 3-keto-pent-4-enoyl-CoA → R/S 3-hydroxy- |
| | dehydrogenase | 4-pentenoyl-CoA |
| J. | 3-hydroxy-4-pentenoyl-CoA | R/S 3-hydroxy-4-pentenoyl-CoA → 3- |
| | transferase | hydroxypent-4-enoic acid |
| | or hydrolase or synthase | |
| K. | 3-hydroxy-4-pentenoic acid | 3-hydroxypent-4-enoic acid → butadiene |
| | decarboxylase | |

In some embodiments, the one or more polynucleotides coding for enzymes in a pathway that catalyze a conversion of oxalacetate to succinate, and a conversion of 3-hydroxypropionyl-CoA to butadiene include:
one or more polynucleotides coding for enzymes in a pathway that catalyze a conversion of PEP to oxaloacetate (e.g., PEP carboxykinase or PEP carboxylase);
one or more polynucleotides coding for enzymes in a pathway that catalyze a conversion of pyruvate to malate (e.g., pyruvate carboxylase);
one or more polynucleotides coding for enzymes in a pathway that catalyze a conversion of oxaloacetate to malate (e.g., malate dehydrogenase);
one or more polynucleotides coding for enzymes in a pathway that catalyze a conversion of malate to fumarate (e.g., fumarase);
one or more polynucleotides coding for enzymes in a pathway that catalyze a conversion of fumarate to succinate (e.g., fumarate reductase);
one or more polynucleotides coding for enzymes in a pathway that catalyze a conversion of pyruvate to R/S lactate (e.g., lactate dehydrogenase);
one or more polynucleotides coding for enzymes in a pathway that catalyze a conversion of R/S lactate to lactoyl-CoA (e.g., lactoyl-CoA transferase or synthase);
one or more polynucleotides coding for enzymes in a pathway that catalyze a conversion of lactoyl-CoA to acryloyl-CoA (e.g., lactoyl-CoA dehydratase);
one or more polynucleotides coding for enzymes in a pathway that catalyze a conversion of acryloyl-CoA to 3-hydroxypropyonyl-CoA (e.g., acryloyl-CoA hydratase);
one or more polynucleotides coding for enzymes in a pathway that catalyze a conversion of acetyl-CoA and 3-hydroxypropionyl-CoA to 5-hydroxy-3-ketovaleryl-CoA (e.g., 5-hydroxy-3-ketovaleryl-CoA thiolase);
one or more polynucleotides coding for enzymes in a pathway that catalyze a conversion of 5-hydroxy-3-Ketovaleryl-CoA and NADH to R/S 3,5-dihydroxy-valeryl-CoA (e.g., 5-hydroxy-3-Ketovaleryl-CoA dehydrogenase);
one or more polynucleotides coding for enzymes in a pathway that catalyze a conversion of R/S 3,5-dihydroxy-valeryl-CoA to R/S 3-hydroxy-4-pentenoyl-CoA (e.g., 3,5-hydroxyvaleryl-CoA dehydratase);
one or more polynucleotides coding for enzymes in a pathway that catalyze a conversion of R/S 3-hydroxy-4-pentenoyl-CoA to 3-hydroxypent-4-enoic acid (e.g., 3-hydroxy-4-pentenoyl-CoA hydrolase, transferase or synthase); and/or
one or more polynucleotides coding for enzymes in a pathway that catalyze a conversion of 3-hydroxypent-4-enoic acid to butadiene (e.g., 3-hydroxy-4-pentenoic acid decarboxylase).

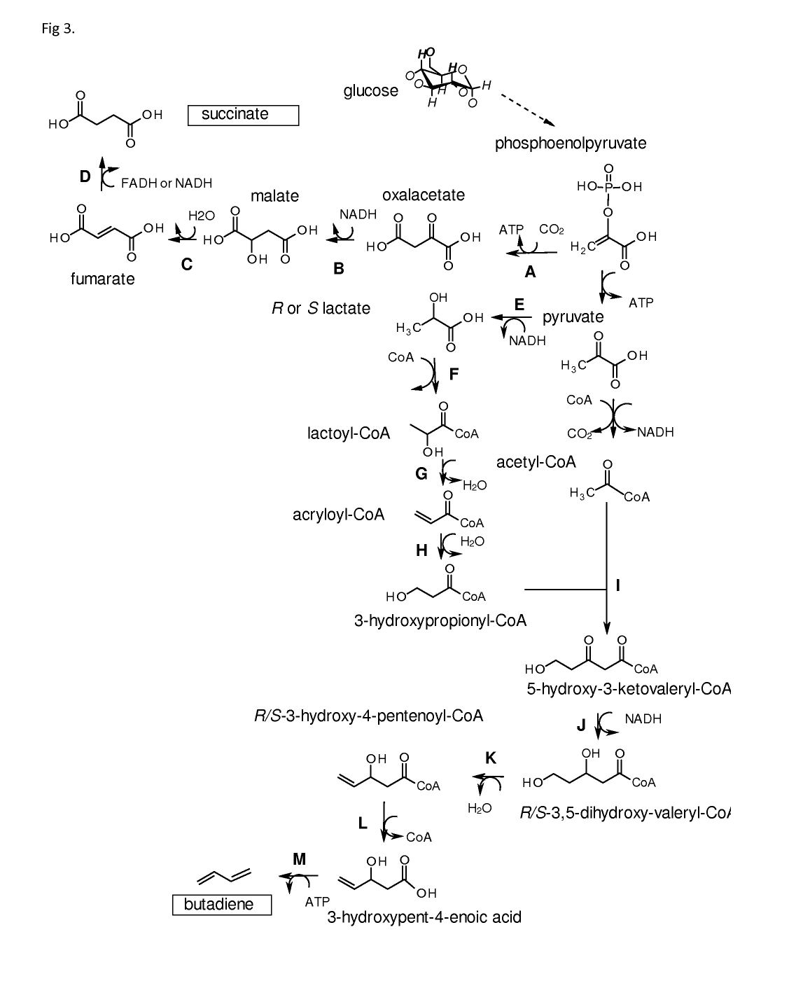

Exemplary enzymes that convert oxalacetate to succinate and 3-hydroxypropionyl-CoA to butadiene including, enzyme substrates, and enzyme reaction products associated with the conversions are presented in Table 3 below. The enzyme reference identifier listed in Table 3 correlates with the enzyme numbering used in FIG. 3, which schematically represents the enzymatic conversion of a fermentable carbon source to succinate and butadiene.

TABLE 3
Production of succinate via an oxalacetate intermediate and butadiene via a
3-hydroxypropionyl-CoA intermediate.

| Enzyme No. | Enzyme Name | Mediated Conversion |
|---|---|---|
| A. | PEP carboxykinase or | PEP → oxaloacetate |
| | PEP carboxylase or | PEP → oxaloacetate |
| | pyruvate carboxylase | pyruvate → malate |
| B. | malate dehydrogenase | oxaloacetate → malate |
| C. | fumarase | malate → fumarate |
| D. | fumarate reductase | fumarate → succinate |
| E. | lactate dehydrogenase | pyruvate → R/S lactate |
| F. | lactoyl-CoA transferase | R/S lactate → lactoyl-CoA |
| | or synthase | |
| G. | lactoyl-CoA dehydratase | lactoyl-CoA → acryloyl-CoA |
| H. | acryloyl-CoA hydratase | acryloyl-CoA →3-hydroxypropyonyl-CoA |
| I. | 5-hydroxy-3-ketovaleryl-CoA | acetyl-CoA + 3-hydroxypropionyl-CoA → 5- |
| | thiolase | hydroxy-3-ketovaleryl-CoA |
| J. | 5-hydroxy-3-Ketovaleryl-CoA | 5-hydroxy-3-ketovaleryl-CoA + NADH → R/S |
| | dehydrogenase | 3,5-dihydroxy-valeryl-CoA |
| K. | 3,5-hydroxyvaleryl-CoA | R/S 3,5-dihydroxy-valeryl-CoA → R/S 3- |
| | dehydratase | hydroxy-4-pentenoyl-CoA |
| L. | 3-hydroxy-4-pentenoyl-CoA | R/S 3-hydroxy-4-pentenoyl-CoA → 3- |
| | hydrolase, transferase or synthase | hydroxypent-4-enoic acid |
| M. | 3-hydroxy-4-pentenoic acid | 3-hydroxypent-4-enoic acid → butadiene |
| | decarboxylase | |

In some embodiments, the one or more polynucleotides coding for enzymes in a pathway that catalyze a conversion of oxalacetate to succinate, and a conversion of formyl-CoA to butadiene include:
one or more polynucleotides coding for enzymes in a pathway that catalyze a conversion of PEP to oxaloacetate (e.g., PEP carboxykinase or PEP carboxylase);
one or more polynucleotides coding for enzymes in a pathway that catalyze a conversion of pyruvate to malate (e.g., pyruvate carboxylase);
one or more polynucleotides coding for enzymes in a pathway that catalyze a conversion of oxaloacetate to malate (e.g., malate dehydrogenase);
one or more polynucleotides coding for enzymes in a pathway that catalyze a conversion of malate to fumarate (e.g., fumarase);
one or more polynucleotides coding for enzymes in a pathway that catalyze a conversion of fumarate to succinate (e.g., fumarate reductase);
one or more polynucleotides coding for enzymes in a pathway that catalyze a conversion of acetyl-CoA to acetoacetyl-CoA (e.g., acetoacetyl-CoA thiolase);
one or more polynucleotides coding for enzymes in a pathway that catalyze a conversion of pyruvate and CoA to acetyl-CoA and formate (e.g., acetyl-CoA:formate C-acetyltransferase);
one or more polynucleotides coding for enzymes in a pathway that catalyze a conversion of CO₂ to formate (e.g., formate dehydrogenase);
one or more polynucleotides coding for enzymes in a pathway that catalyze a conversion of formate to formyl-CoA (e.g., formyl-CoA transferase formyl-CoA synthase);
one or more polynucleotides coding for enzymes in a pathway that catalyze a conversion of acetoacetyl-CoA and formyl-CoA to 3,5-ketovaleryl-CoA (e.g., 3,5-ketovaleryl-CoA thiolase);
one or more polynucleotides coding for enzymes in a pathway that catalyze a conversion of 3,5-ketovaleryl-CoA to 5-hydroxy-3-ketovaleryl-CoA (e.g., 5-hydroxy-3-ketovaleryl-CoA dehydrogenase);
one or more polynucleotides coding for enzymes in a pathway that catalyze a conversion of 3,5-ketovaleryl-CoA to R/S 5-keto-3-hydroxyvaleryl-CoA (e.g., 3-hydroxy-5-ketovaleryl-CoA dehydrogenase);
one or more polynucleotides coding for enzymes in a pathway that catalyze a conversion of 5-hydroxy-3-ketovaleryl-CoA or R/S 5-keto-3-hydroxyvaleryl-CoA to R/S 3,5-dihydroxy-valeryl-CoA (e.g., 3,5-hydroxyaleryl-CoA dehydrogenase);
one or more polynucleotides coding for enzymes in a pathway that catalyze a conversion of R/S 3,5-dihydroxy-valeryl-CoA to R/S 3-hydroxy-4-pentenoyl-CoA (e.g., 3,5-hydroxyvaleryl-CoA dehydratase);
one or more polynucleotides coding for enzymes in a pathway that catalyze a conversion of R/S 3-hydroxy-4-pentenoyl-CoA to 3-hydroxypent-4-enoic acid (e.g., 3-hydroxy-4-pentenoyl-CoA hydrolase, transferase or synthase); and/or
one or more polynucleotides coding for enzymes in a pathway that catalyze a conversion of 3-hydroxypent-4-enoic acid to butadiene (e.g., 3-hydroxy-4-pentenoic acid decarboxylase).

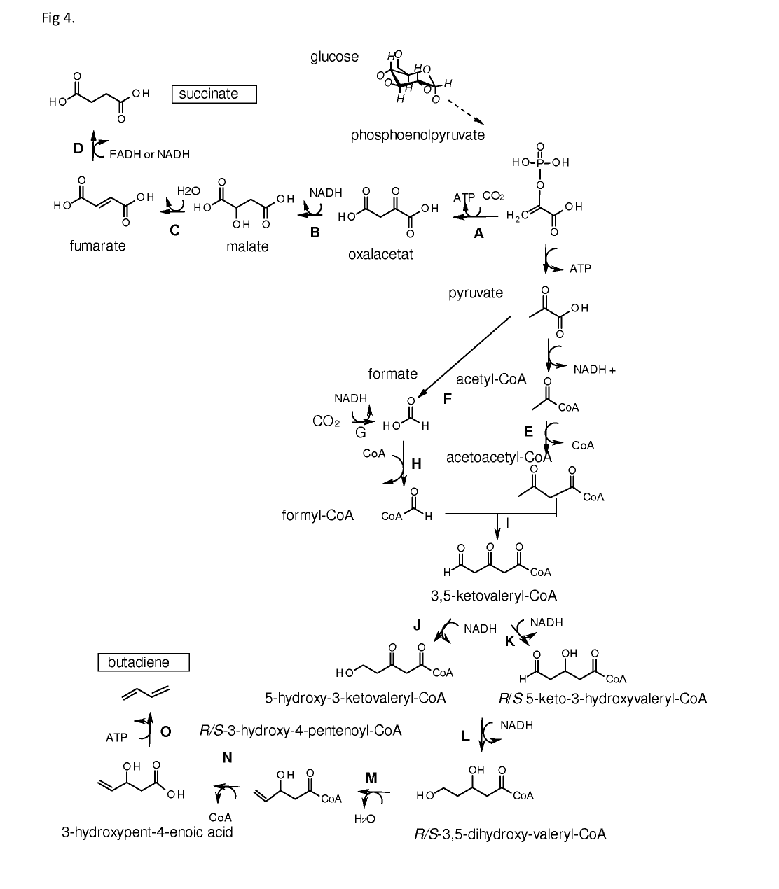

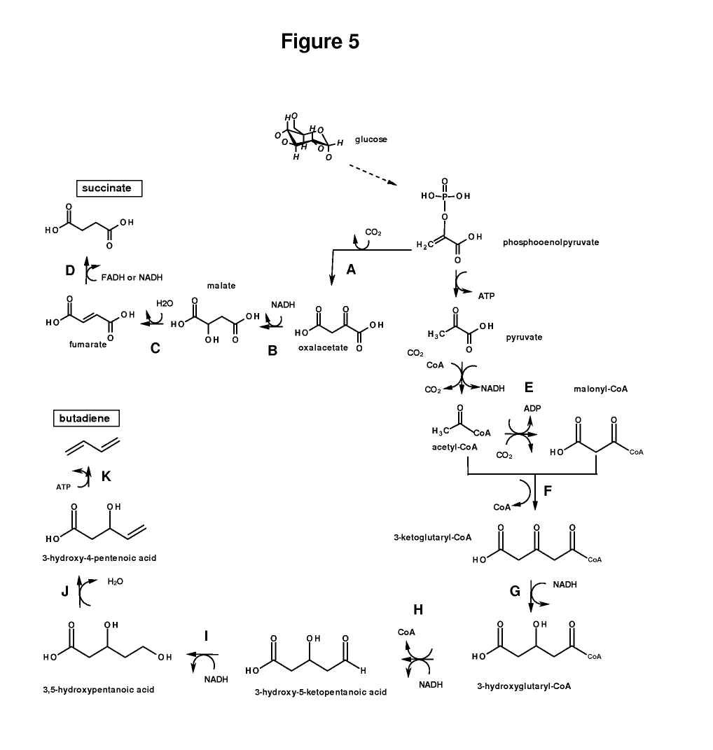

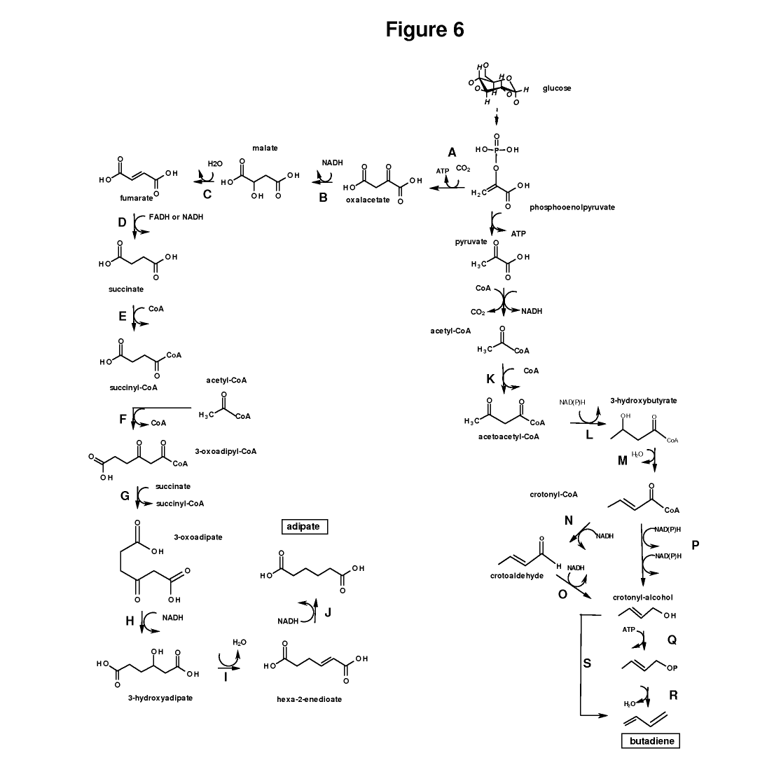

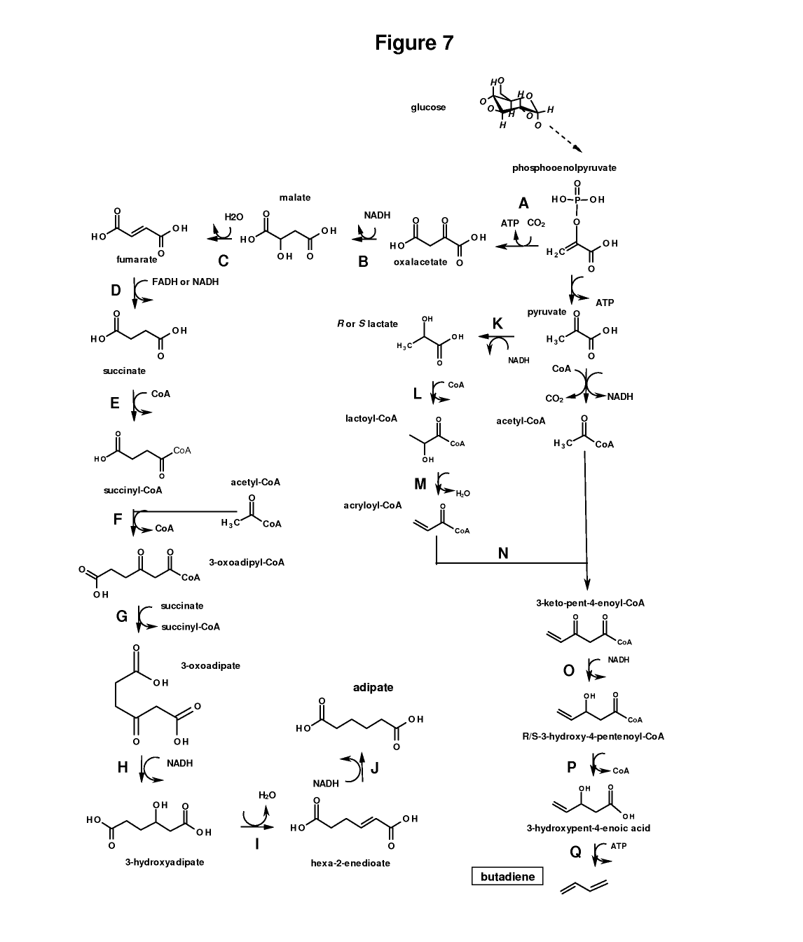

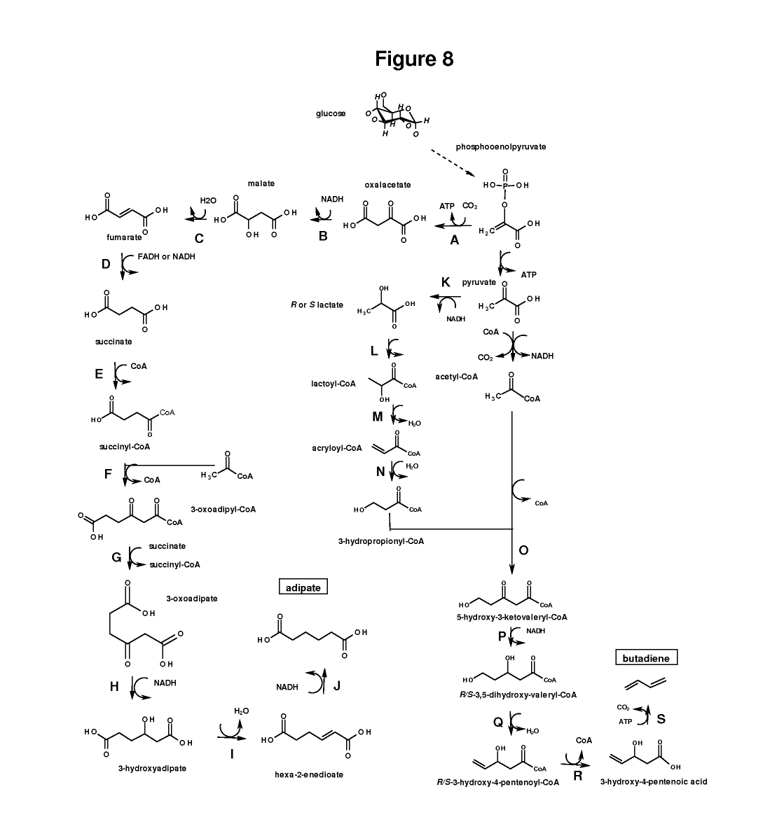

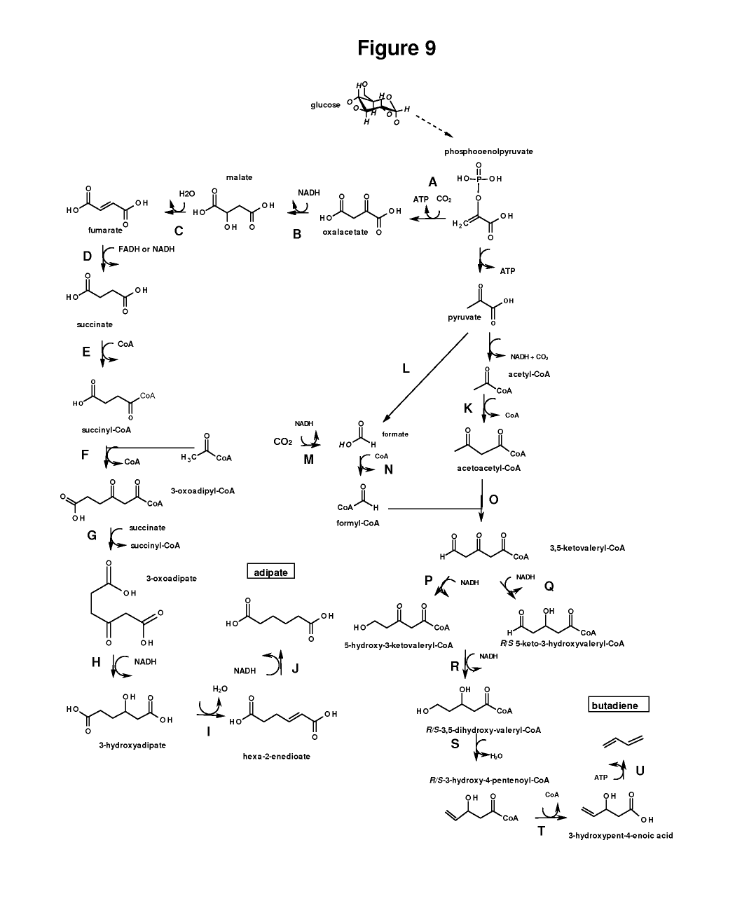

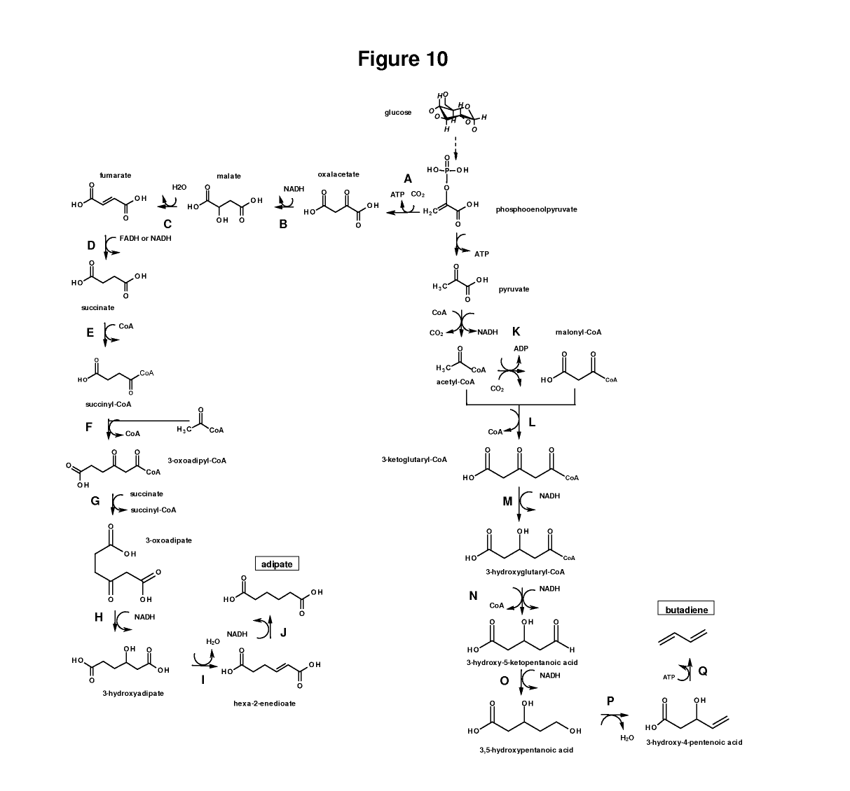

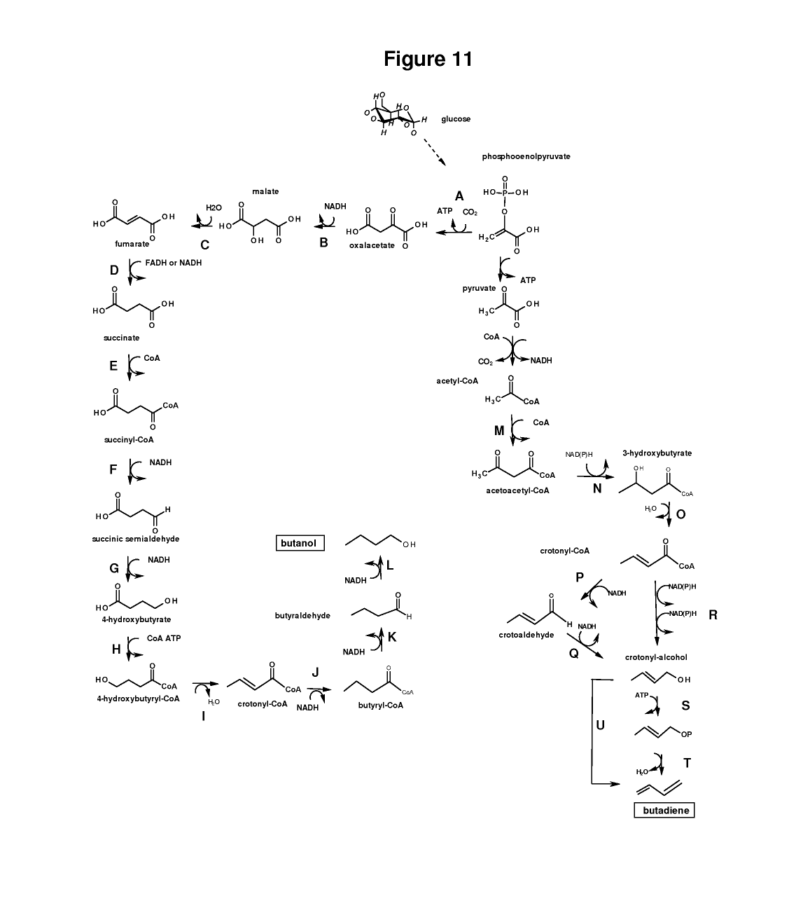

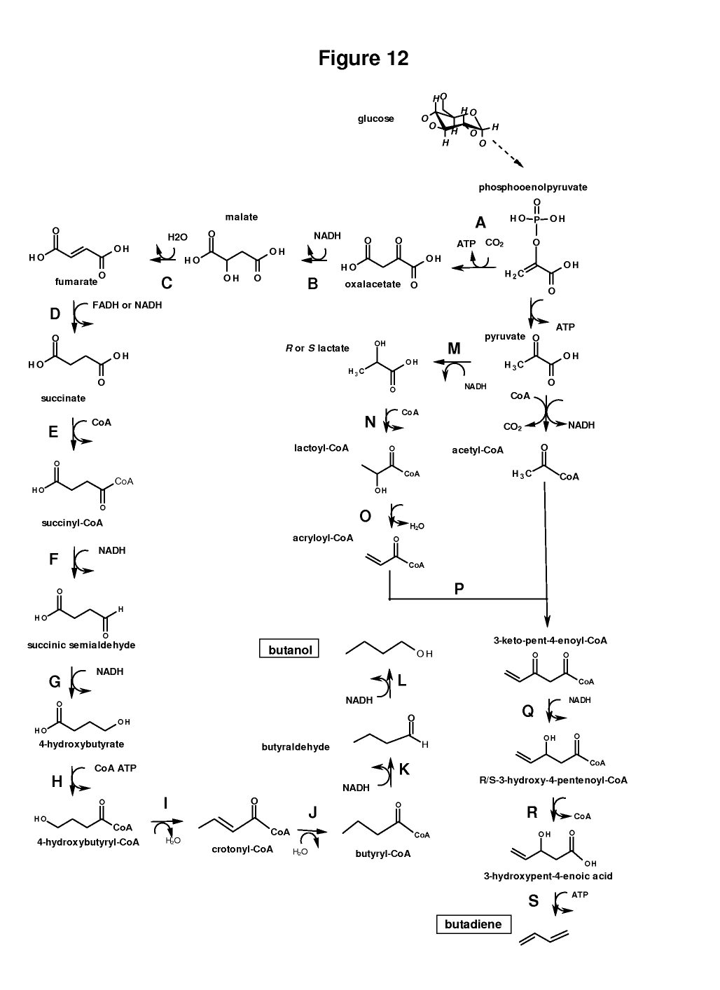

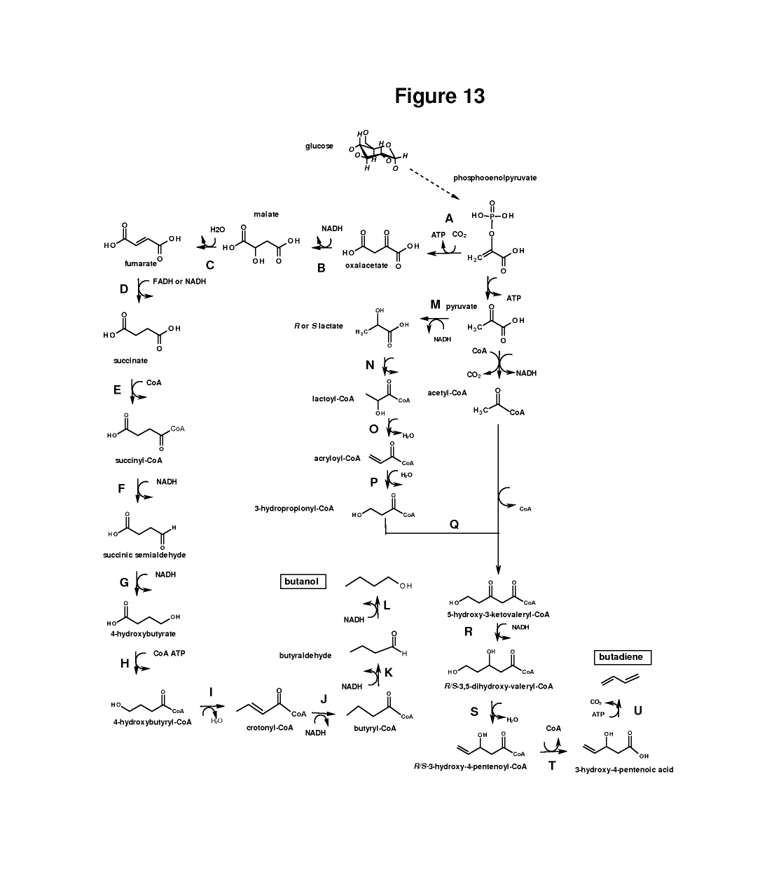

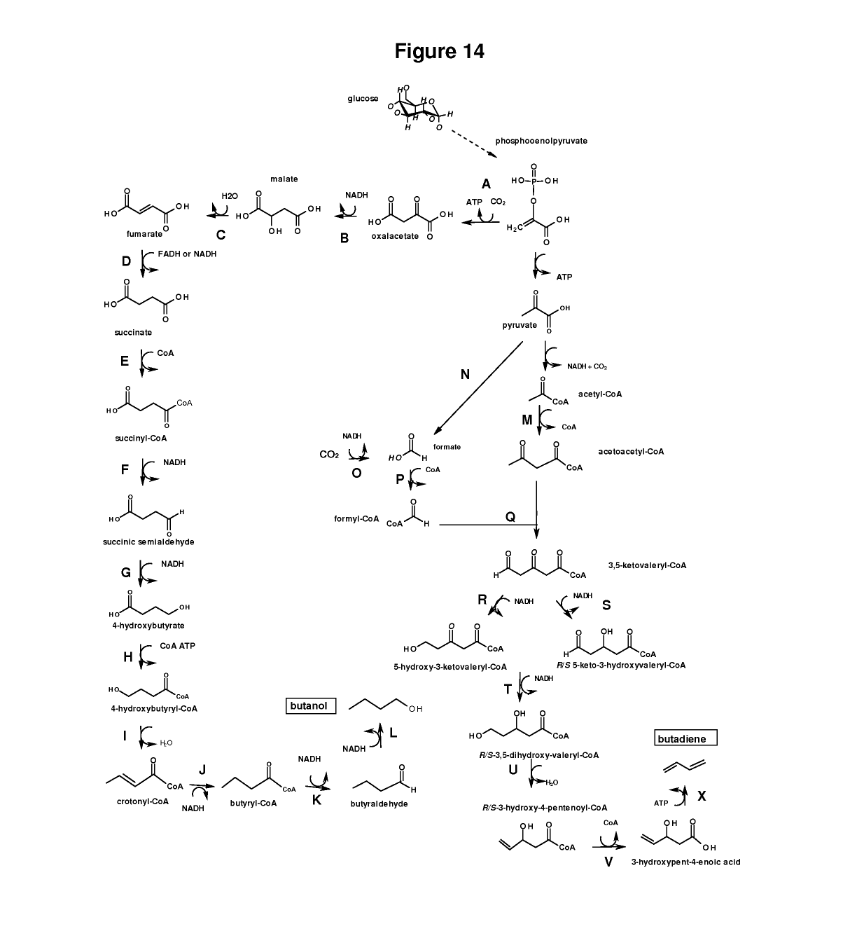

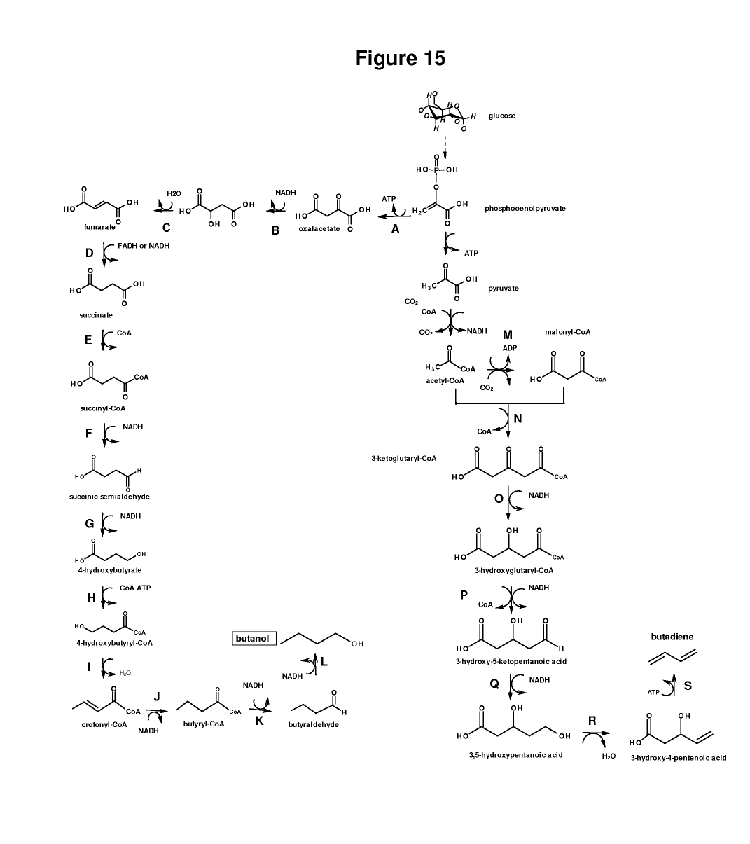

Exemplary enzymes that convert oxalacetate to succinate and formyl-CoA to butadiene including, enzyme substrates, and enzyme reaction products associated with the conversions are presented in Table 4 below. The enzyme reference identifier listed in Table 4 correlates with the enzyme numbering used in FIG. 4, which schematically represents the enzymatic conversion of a fermentable carbon source to succinate and butadiene.

TABLE 4
Production of succinate via an oxalacetate intermediate and butadiene
via a formyl-CoA intermediate.

| Enzyme No. | Enzyme Name | Mediated Conversion |
|---|---|---|
| A. | PEP carboxykinase or | PEP → oxaloacetate |
| | PEP carboxylase or | PEP → oxaloacetate |
| | pyruvate carboxylase | pyruvate → malate |
| B. | malate dehydrogenase | oxaloacetate → malate |
| C. | fumarase | malate → fumarate |
| D. | fumarate reductase | fumarate → succinate |
| E. | acetoacetyl-CoA thiolase | acetyl-CoA → acetoacetyl-CoA |
| F. | acetyltransferase | pyruvate + CoA → acetyl-CoA + Formate |
| G. | formate dehydrogenase | CO₂ → formate |
| H. | formyl-CoA transferase | formate → formyl-CoA |
| | formyl-CoA synthase | |
| I. | 3,5-ketovaleryl-CoA thiolase | acetoacetyl-CoA + formyl-CoA → 3,5- |
| | | ketovaleryl-CoA |
| J. | 5-hydroxy-3-Ketovaleryl-CoA | 3,5-ketovaleryl-CoA → 5-hydroxy-3- |
| | dehydrogenase | ketovaleryl-CoA |
| K. | 3-hydroxy-5-Ketovaleryl-CoA | 3,5-ketovaleryl-CoA → R/S 5-keto-3- |
| | dehydrogenase | hydroxyvaleryl-CoA |
| L. | 3,5-hydroxyaleryl-CoA | 5-hydroxy-3-ketovaleryl-CoA or R/S 5-keto- |
| | dehydrogenase | 3-hydroxyvaleryl-CoA → R/S 3,5-dihydroxy- |
| | | valeryl-CoA |
| M. | 3,5-hydroxyvaleryl-CoA | R/S 3,5-dihydroxy-valeryl-CoA→ R/S 3- |
| | dehydratase | hydroxy-4-pentenoyl-CoA |
| N. | 3-hydroxy-4-pentenoyl-CoA | R/S 3-hydroxy-4-pentenoyl-CoA → 3- |
| | hydrolase, transferase or synthase | hydroxypent-4-enoic acid |
| O. | 3-hydroxy-4-pentenoic acid | 3-hydroxypent-4-enoic acid → butadiene |
| | decarboxylase | |

The microorganism may be an archea, bacteria, or eukaryote. In some embodiments, the bacteria is a Propionibacterium, Propionispira, Clostridium, Bacillus, Escherichia, Pelobacter, or Lactobacillus including, for example, Pelobacter propionicus, Clostridium propionicum, Clostridium acetobutylicum, Lactobacillus, Propionibacterium acidipropionici or Propionibacterium freudenreichii. In some embodiments, the eukaryote is a yeast, filamentous fungi, protozoa, or algae. In some embodiments, the yeast is Saccharomyces cerevisiae or Pichia pastoris.

In some embodiments, the microorganism is additionally modified to comprise one or more tolerance mechanisms including, for example, tolerance to a produced biofuel, and/or organic solvents. A microorganism modified to comprise such a tolerance mechanism may provide a means to increase titers of fermentations and/or may control contamination in an industrial scale process.

In some embodiments, the disclosure contemplates the modification (e.g., engineering) of one or more of the enzymes provided herein. Such modification may be performed to redesign the substrate specificity of the enzyme and/or to modify (e.g., reduce) its activity against others substrates in order to increase its selectivity for a given substrate. Additionally or alternatively, one or more enzymes as provided herein may be engineered to alter (e.g., enhance including, for example, increase its catalytic activity or its substrate specificity) one or more of its properties.

In some embodiments, sequence alignment and comparative modeling of proteins may be used to alter one or more of the enzymes disclosed herein. Homology modeling or comparative modeling refers to building an atomic-resolution model of the desired protein from its primary amino acid sequence and an experimental three-dimensional structure of a similar protein. This model may allow for the enzyme substrate binding site to be defined, and the identification of specific amino acid positions that may be replaced to other natural amino acid in order to redesign its substrate specificity.

Variants or sequences having substantial identity or homology with the polynucleotides encoding enzymes as disclosed herein may be utilized in the practice of the disclosure. Such sequences can be referred to as variants or modified sequences. That is, a polynucleotide sequence may be modified yet still retain the ability to encode a polypeptide exhibiting the desired activity. Such variants or modified sequences are thus equivalents in the sense that they retain their intended function. Generally, the variant or modified sequence may comprise at least about 40%-60%, preferably about 60%-80%, more preferably about 80%-90%, and even more preferably about 90%-95% sequence identity with the native sequence.

In some embodiments, a microorganism may be modified to express including, for example, overexpress, one or more enzymes as provided herein. The microorganism may be modified by genetic engineering techniques (i.e., recombinant technology), classical microbiological techniques, or a combination of such techniques and can also include naturally occurring genetic variants to produce a genetically modified microorganism. Some of such techniques are generally disclosed, for example, in Sambrook et al., 1989, Molecular Cloning: A Laboratory Manual, Cold Spring Harbor Labs Press.

A genetically modified microorganism may include a microorganism in which a polynucleotide has been inserted, deleted or modified (i.e., mutated; e.g., by insertion, deletion, substitution, and/or inversion of nucleotides), in such a manner that such modifications provide the desired effect of expression (e.g., over-expression) of one or more enzymes as provided herein within the microorganism. Genetic modifications which result in an increase in gene expression or function can be referred to as amplification, overproduction, overexpression, activation, enhancement, addition, or up-regulation of a gene. Addition of cloned genes to increase gene expression can include maintaining the cloned gene(s) on replicating plasmids or integrating the cloned gene(s) into the genome of the production organism. Furthermore, increasing the expression of desired cloned genes can include operatively linking the cloned gene(s) to native or heterologous transcriptional control elements.

Where desired, the expression of one or more of the enzymes provided herein are under the control of a regulatory sequence that controls directly or indirectly the expression of the enzyme in a time-dependent fashion during a fermentation reaction.

In some embodiments, a microorganism is transformed or transfected with a genetic vehicle such as, an expression vector comprising an exogenous polynucleotide sequence coding for the enzymes provided herein.

Polynucleotide constructs prepared for introduction into a prokaryotic or eukaryotic host may typically, but not always, comprise a replication system (i.e. vector) recognized by the host, including the intended polynucleotide fragment encoding the desired polypeptide, and may preferably, but not necessarily, also include transcription and translational initiation regulatory sequences operably linked to the polypeptide-encoding segment. Expression systems (expression vectors) may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, mRNA stabilizing sequences, nucleotide sequences homologous to host chromosomal DNA, and/or a multiple cloning site. Signal peptides may also be included where appropriate, preferably from secreted polypeptides of the same or related species, which allow the protein to cross and/or lodge in cell membranes or be secreted from the cell.

The vectors can be constructed using standard methods (see, e.g., Sambrook et al., Molecular Biology: A Laboratory Manual, Cold Spring Harbor, N.Y. 1989; and Ausubel, et al., Current Protocols in Molecular Biology, Greene Publishing, Co. N.Y, 1995).

The manipulation of polynucleotides of the present disclosure including polynucleotides coding for one or more of the enzymes disclosed herein is typically carried out in recombinant vectors. Numerous vectors are publicly available, including bacterial plasmids, bacteriophage, artificial chromosomes, episomal vectors and gene expression vectors, which can all be employed. A vector of use according to the disclosure may be selected to accommodate a protein coding sequence of a desired size. A suitable host cell is transformed with the vector after in vitro cloning manipulations. Host cells may be prokaryotic, such as any of a number of bacterial strains, or may be eukaryotic, such as yeast or other fungal cells, insect or amphibian cells, or mammalian cells including, for example, rodent, simian or human cells. Each vector contains various functional components, which generally include a cloning site, an origin of replication and at least one selectable marker gene. If given vector is an expression vector, it additionally possesses one or more of the following: enhancer element, promoter, transcription termination and signal sequences, each positioned in the vicinity of the cloning site, such that they are operatively linked to the gene encoding a polypeptide repertoire member according to the disclosure.

Vectors, including cloning and expression vectors, may contain nucleic acid sequences that enable the vector to replicate in one or more selected host cells. For example, the sequence may be one that enables the vector to replicate independently of the host chromosomal DNA and may include origins of replication or autonomously replicating sequences. Such sequences are well known for a variety of bacteria, yeast and viruses. For example, the origin of replication from the plasmid pBR322 is suitable for most Gram-negative bacteria, the 2 micron plasmid origin is suitable for yeast, and various viral origins (e.g. SV 40, adenovirus) are useful for cloning vectors in mammalian cells. Generally, the origin of replication is not needed for mammalian expression vectors unless these are used in mammalian cells able to replicate high levels of DNA, such as COS cells.

A cloning or expression vector may contain a selection gene also referred to as a selectable marker. This gene encodes a protein necessary for the survival or growth of transformed host cells grown in a selective culture medium. Host cells not transformed with the vector containing the selection gene will therefore not survive in the culture medium. Typical selection genes encode proteins that confer resistance to antibiotics and other toxins, e.g. ampicillin, neomycin, methotrexate, hygromycin, thiostrepton, apramycin or tetracycline, complement auxotrophic deficiencies, or supply critical nutrients not available in the growth media.

The replication of vectors may be performed in E. coli (e.g., strain TB1 or TG1, DH5α, DH10β, JM110). An E. coli-selectable marker, for example, the β-lactamase gene that confers resistance to the antibiotic ampicillin, may be of use. These selectable markers can be obtained from E. coli plasmids, such as pBR322 or a pUC plasmid such as pUC18 or pUC19, or pUC119.

Expression vectors may contain a promoter that is recognized by the host organism. The promoter may be operably linked to a coding sequence of interest. Such a promoter may be inducible or constitutive. Polynucleotides are operably linked when the polynucleotides are in a relationship permitting them to function in their intended manner.

Promoters suitable for use with prokaryotic hosts may include, for example, the α-lactamase and lactose promoter systems, alkaline phosphatase, the tryptophan (trp) promoter system, the erythromycin promoter, apramycin promoter, hygromycin promoter, methylenomycin promoter and hybrid promoters such as the tac promoter. Moreover, host constitutive or inducible promoters may be used. Promoters for use in bacterial systems will also generally contain a Shine-Dalgarno sequence operably linked to the coding sequence.

Viral promoters obtained from the genomes of viruses include promoters from polyoma virus, fowlpox virus, adenovirus (e.g., Adenovirus 2 or 5), herpes simplex virus (thymidine kinase promoter), bovine papilloma virus, avian sarcoma virus, cytomegalovirus, a retrovirus (e.g., MoMLV, or RSV LTR), Hepatitis-B virus, Myeloproliferative sarcoma virus promoter (MPSV), VISNA, and Simian Virus 40 (SV40). Heterologous mammalian promoters include, e.g., the actin promoter, immunoglobulin promoter, heat-shock protein promoters.

The early and late promoters of the SV40 virus are conveniently obtained as a restriction fragment that also contains the SV40 viral origin of replication (see, e.g., Fiers et al., Nature, 273:113 (1978); Mulligan and Berg, Science, 209:1422-1427 (1980); and Pavlakis et al., Proc. Natl. Acad. Sci. USA, 78:7398-7402 (1981)). The immediate early promoter of the human cytomegalovirus (CMV) is conveniently obtained as a Hind III E restriction fragment (see, e.g., Greenaway et al., Gene, 18:355-360 (1982)). A broad host range promoter, such as the SV40 early promoter or the Rous sarcoma virus LTR, is suitable for use in the present expression vectors.

Generally, a strong promoter may be employed to provide for high level transcription and expression of the desired product. Among the eukaryotic promoters that have been identified as strong promoters for high-level expression are the SV40 early promoter, adenovirus major late promoter, mouse metallothionein-I promoter, Rous sarcoma virus long terminal repeat, and human cytomegalovirus immediate early promoter (CMV or CMV IE). In an embodiment, the promoter is a SV40 or a CMV early promoter.

The promoters employed may be constitutive or regulatable, e.g., inducible. Exemplary inducible promoters include jun, fos and metallothionein and heat shock promoters. One or both promoters of the transcription units can be an inducible promoter. In an embodiment, the GFP is expressed from a constitutive promoter while an inducible promoter drives transcription of the gene coding for one or more enzymes as disclosed herein and/or the amplifiable selectable marker.

The transcriptional regulatory region in higher eukaryotes may comprise an enhancer sequence. Many enhancer sequences from mammalian genes are known e.g., from globin, elastase, albumin, α-fetoprotein and insulin genes. A suitable enhancer is an enhancer from a eukaryotic cell virus. Examples include the SV40 enhancer on the late side of the replication origin (bp 100-270), the enhancer of the cytomegalovirus immediate early promoter (Boshart et al. Cell 41:521 (1985)), the polyoma enhancer on the late side of the replication origin, and adenovirus enhancers (see also, e.g., Yaniv, Nature, 297:17-18 (1982) on enhancing elements for activation of eukaryotic promoters). The enhancer sequences may be introduced into the vector at a position 5′ or 3′ to the gene of interest, but is preferably located at a site 5′ to the promoter.

Yeast and mammalian expression vectors may contain prokaryotic sequences that facilitate the propagation of the vector in bacteria. Therefore, the vector may have other components such as an origin of replication (e.g., a nucleic acid sequence that enables the vector to replicate in one or more selected host cells), antibiotic resistance genes for selection in bacteria, and/or an amber stop codon which can permit translation to read through the codon. Additional eukaryotic selectable gene(s) may be incorporated. Generally, in cloning vectors the origin of replication is one that enables the vector to replicate independently of the host chromosomal DNA, and includes origins of replication or autonomously replicating sequences. Such sequences are well known, e.g., the ColE1 origin of replication in bacteria. Various viral origins (e.g., SV40, polyoma, adenovirus, VSV or BPV) are useful for cloning vectors in mammalian cells. Generally, a eukaryotic replicon is not needed for expression in mammalian cells unless extrachromosomal (episomal) replication is intended (e.g., the SV40 origin may typically be used only because it contains the early promoter).

To facilitate insertion and expression of different genes coding for the enzymes as disclosed herein from the constructs and expression vectors, the constructs may be designed with at least one cloning site for insertion of any gene coding for any enzyme disclosed herein. The cloning site may be a multiple cloning site, e.g., containing multiple restriction sites.

The plasmids may be propagated in bacterial host cells to prepare DNA stocks for subcloning steps or for introduction into eukaryotic host cells. Transfection of eukaryotic host cells can be any performed by any method well known in the art. Transfection methods include lipofection, electroporation, calcium phosphate co-precipitation, rubidium chloride or polycation mediated transfection, protoplast fusion and microinjection. Preferably, the transfection is a stable transfection. The transfection method that provides optimal transfection frequency and expression of the construct in the particular host cell line and type, is favored. Suitable methods can be determined by routine procedures. For stable transfectants, the constructs are integrated so as to be stably maintained within the host chromosome.

Vectors may be introduced to selected host cells by any of a number of suitable methods known to those skilled in the art. For example, vector constructs may be introduced to appropriate cells by any of a number of transformation methods for plasmid vectors. For example, standard calcium-chloride-mediated bacterial transformation is still commonly used to introduce naked DNA to bacteria (see, e.g., Sambrook et al., 1989, Molecular Cloning, A Laboratory Manual, Cold Spring Harbor Laboratory Press, Cold Spring Harbor, N.Y.), but electroporation and conjugation may also be used (see, e.g., Ausubel et al., 1988, Current Protocols in Molecular Biology, John Wiley & Sons, Inc., NY, N.Y.).

For the introduction of vector constructs to yeast or other fungal cells, chemical transformation methods may be used (e.g., Rose et al., 1990, Methods in Yeast Genetics, Cold Spring Harbor Laboratory Press, Cold Spring Harbor, N.Y.). Transformed cells may be isolated on selective media appropriate to the selectable marker used. Alternatively, or in addition, plates or filters lifted from plates may be scanned for GFP fluorescence to identify transformed clones.

For the introduction of vectors comprising differentially expressed sequences to mammalian cells, the method used may depend upon the form of the vector. Plasmid vectors may be introduced by any of a number of transfection methods, including, for example, lipid-mediated transfection (“lipofection”), DEAE-dextran-mediated transfection, electroporation or calcium phosphate precipitation (see, e.g., Ausubel et al., 1988, Current Protocols in Molecular Biology, John Wiley & Sons, Inc., NY, N.Y.).

Lipofection reagents and methods suitable for transient transfection of a wide variety of transformed and non-transformed or primary cells are widely available, making lipofection an attractive method of introducing constructs to eukaryotic, and particularly mammalian cells in culture. For example, LipofectAMINE™ (Life Technologies) or LipoTaxi™ (Stratagene) kits are available. Other companies offering reagents and methods for lipofection include Bio-Rad Laboratories, CLONTECH, Glen Research, InVitrogen, JBL Scientific, MBI Fermentas, PanVera, Promega, Quantum Biotechnologies, Sigma-Aldrich, and Wako Chemicals USA.

The host cell may be capable of expressing the construct encoding the desired protein, processing the protein and transporting a secreted protein to the cell surface for secretion. Processing includes co- and post-translational modification such as leader peptide cleavage, GPI attachment, glycosylation, ubiquitination, and disulfide bond formation. Immortalized host cell cultures amenable to transfection and in vitro cell culture and of the kind typically employed in genetic engineering are preferred. Examples of useful mammalian host cell lines are monkey kidney CV1 line transformed by SV40 (COS-7, ATCC CRL 1651); human embryonic kidney line (293 or 293 derivatives adapted for growth in suspension culture, Graham et al., J. Gen Virol., 36:59 (1977); baby hamster kidney cells (BHK, ATCC CCL 10); DHFR-Chinese hamster ovary cells (ATCC CRL-9096); dp12.CHO cells, a derivative of CHO/DHFR-(EP 307,247 published 15 Mar. 1989); mouse sertoli cells (TM4, Mather, Biol. Reprod., 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 (1982)); PEER human acute lymphoblastic cell line (Ravid et al. Int. J. Cancer 25:705-710 (1980)); MRC 5 cells; FS4 cells; human hepatoma line (Hep G2), human HT1080 cells, KB cells, JW-2 cells, Detroit 6 cells, NIH-3T3 cells, hybridoma and myeloma cells. Embryonic cells used for generating transgenic animals are also suitable (e.g., zygotes and embryonic stem cells).

Suitable host cells for cloning or expressing polynucleotides (e.g., DNA) in vectors may include, for example, prokaryote, yeast, or higher eukaryote cells. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. lichenifonnis (e.g., B. lichenifonnis 41 P disclosed in DD 266,710 published Apr. 12, 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), E. coli JM110 (ATCC 47,013) and E. coli W3110 (ATCC 27,325) are suitable.

In addition to prokaryotes, eukaryotic microbes such as filamentous fungi or yeast may be suitable cloning or expression hosts for vectors comprising polynucleotides coding for one or more enzymes. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastors (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

When the enzyme is glycosylated, suitable host cells for expression may be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori (silk moth) have been identified. A variety of viral strains for transfection are publicly available, e.g., the L-1 variant of Autographa californica NPV and the Bm-5 strain of Bombyx mori NPV, and such viruses may be used as the virus herein according to the present disclosure, particularly for transfection of Spodoptera frugiperda cells.

Plant cell cultures of cotton, corn, potato, soybean, petunia, tomato, tobacco, lemna, and other plant cells can also be utilized as host cells.

Examples of useful mammalian host cells are Chinese hamster ovary cells, including CHOK1 cells (ATCC CCL61), DXB-11, DG-44, and Chinese hamster ovary cells/-DHFR (CHO, Urlaub et al., Proc. Natl. Acad. Sci. USA 77: 4216 (1980)); monkey kidney CV1 line transformed by SV40 (COS-7, ATCC CRL 1651); human embryonic kidney line (293 or 293 cells subcloned for growth in suspension culture, (Graham et al., J. Gen Virol. 36: 59, 1977); baby hamster kidney cells (BHK, ATCC CCL 10); mouse sertoli cells (TM4, Mather, (Biol. Reprod. 23: 243-251, 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383: 44-68 (1982)); MRC 5 cells; FS4 cells; and a human hepatoma line (Hep G2).

Host cells are transformed or transfected with the above-described expression or cloning vectors for production of one or more enzymes as disclosed herein or with polynucleotides coding for one or more enzymes as disclosed herein and cultured in conventional nutrient media modified as appropriate for inducing promoters, selecting transformants, or amplifying the genes encoding the desired sequences.

Host cells containing desired nucleic acid sequences coding for the disclosed enzymes may be cultured in a variety of media. Commercially available media such as Ham's F10 (Sigma), Minimal Essential Medium ((MEM), Sigma), RPMI-1640 (Sigma), and Dulbecco's Modified Eagle's Medium ((DMEM), Sigma) are suitable for culturing the host cells. In addition, any of the media described in Ham et al., Meth. Enz. 58: 44, (1979); Barnes et al., Anal. Biochem. 102: 255 (1980); U.S. Pat. Nos. 4,767,704; 4,657,866; 4,927,762; 4,560,655; or 5,122,469; WO90103430; WO 87/00195; or U.S. Pat. Re. No. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adeNOSine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

### Polynucleotides and Encoded Enzymes

Any known polynucleotide (e.g., gene) that codes for an enzyme or variant thereof that is capable of catalyzing an enzymatic conversion including, for example, an enzyme as set forth in any one of Tables 1-15 or FIGS. 1-15, is contemplated for use by the present disclosure. Such polynucleotides may be modified (e.g., genetically engineered) to modulate (e.g., increase or decrease) the substrate specificity of an encoded enzyme, or the polynucleotides may be modified to change the substrate specificity of the encoded enzyme (e.g., a polynucleotide that codes for an enzyme with specificity for a substrate may be modified such that the enzyme has specificity for an alternative substrate). Preferred microorganisms may comprise polynucleotides coding for one or more of the enzymes as set forth in any one of Tables 1-15 and FIG. 1-15.

Enzymes, and polynucleotides encoding same, for catalyzing the conversions in Tables 1-4 and FIGS. 1-4 are categorized in Table 16-19, respectively, by Enzyme Commission (EC) number, function, and the step in Tables 1-4 and FIGS. 1-4 in which they catalyze a conversion.

TABLE 5
Exemplary genes coding for enzymes in Table 1 and FIG. 1.

| | | | | Gene ID (GI) or | |
|---|---|---|---|---|---|
| Enzyme | | | | Accession Number | SEQ ID |
| No. | EC No. | Gene | Organism | (AN) | NO: |
| A | 4.1.1.49 | pckA | Escherichia coli | 12933187 | 1 |
| A | 4.1.1.49 | pckA | Actinobacillus | 5349670 | 2 |
| | | | succinogenes | | |
| B | 1.1.1.37 | mdh1 | Escherichia coli | 12931785 | 3 |
| B | 1.1.1.37 | mdh2 | Saccharomyces | 853994 | 4 |
| | | | cerevisiae | | |
| B | 1.1.5.4 | mqo | Escherichia coli | 12933223 | 5 |
| C | 4.2.1.2 | fum1 | Saccharomyces | 855866 | 6 |
| | | | cerevisiae | | |
| C | 4.2.1.2 | fumA | Escherichia coli | 12934128 | 7 |
| C | 4.2.1.2 | fumB | Escherichia coli | 12933156 | 8 |
| C | 4.2.1.2 | fumC | Escherichia coli | 12934129 | 9 |
| D | 1.3.99.1 | frdA | Escherichia coli | 12931889 | 10 |
| D | 1.3.99.1 | frdB | Escherichia coli | 12933707 | 11 |
| D | 1.3.99.1 | frdC | Escherichia coli | 12933706 | 12 |
| D | 1.3.99.1 | frdD | Escherichia coli | 12933705 | 13 |
| D | 1.3.5.1 | sdhA | Escherichia coli | 12933913 | 14 |
| D | 1.3.5.1 | sdhB | Escherichia coli | 12932956 | 15 |
| D | 1.3.5.1 | sdhC | Escherichia coli | 12930948 | 16 |
| D | 1.3.5.1 | sdhD | Escherichia coli | 12930949 | 17 |
| F | 1.1.1.157 | hbd | Clostridium | 1118891 | 18 |
| | | | acetobutylicum | | |
| F | 1.1.1.35 | hbd | Clostridium | 20162442 | 19 |
| | | | beijerinckii | | |
| G | 4.2.1.55 | crt | Clostridium | 1118895 | 20 |
| | | | acetobutylicum | | |
| J | 1.2.1.10/1.1.1.1 | adhE2 | Clostridium | 12958625 | 21 |
| | | | acetobutylicum | | |
| H | 1.2.1. | Ald6p | Saccharomyces | 856044 | 22 |
| | | | cerevisiae | | |
| H | 1.2.1.10 | mhpF | Escherichia coli | 12932628 | 23 |
| K | 4.2.1.127 | ldi | Castellaniella | 302064203 | 24 |
| | | | defragrans | | |
| L | 2.7.1.36 | erg12 | Saccharomyces | 855248 | 25 |
| | | | cerevisiae | | |
| M | 2.7.4.2 | erg8 | Saccharomyces | 855260 | 26 |
| | | | cerevisiae | | |
| O | 2.7.1.33 | coaA | Escherichia coli | 12934389 | 27 |
| N | 4.2.3.27 | mgsA | Bacillus subtilis | 16079305 | 28 |

TABLE 6
Exemplary genes coding for enzymes in Table 2 and FIG. 2.

| | | | | Gene ID (GI) or | |
|---|---|---|---|---|---|
| Enzyme | | | | Accession Number | SEQ ID |
| No. | EC No. | Gene | Organism | (AN) | NO: |
| A | 4.1.1.49 | pckA | Escherichia coli | 12933187 | 29 |
| A | 4.1.1.49 | pckA | Actinobacillus | 5349670 | 30 |
| | | | succinogenes | | |
| B | 1.1.1.37 | mdh1 | Escherichia coli | 12931785 | 31 |
| B | 1.1.1.37 | mdh2 | Saccharomyces | 853994 | 32 |
| | | | cerevisiae | | |
| B | 1.1.5.4 | mqo | Escherichia coli | 12933223 | 33 |
| C | 4.2.1.2 | fum1 | Saccharomyces | 855866 | 34 |
| | | | cerevisiae | | |
| C | 4.2.1.2 | fumA | Escherichia coli | 12934128 | 35 |
| C | 4.2.1.2 | fumB | Escherichia coli | 12933156 | 36 |
| C | 4.2.1.2 | fumC | Escherichia coli | 12934129 | 37 |
| D | 1.3.99.1 | frdA | Escherichia coli | 12931889 | 38 |
| D | 1.3.99.1 | frdB | Escherichia coli | 12933707 | 39 |
| D | 1.3.99.1 | frdC | Escherichia coli | 12933706 | 40 |
| D | 1.3.99.1 | frdD | Escherichia coli | 12933705 | 41 |
| D | 1.3.5.1 | sdhA | Escherichia coli | 12933913 | 42 |
| D | 1.3.5.1 | sdhB | Escherichia coli | 12932956 | 43 |
| D | 1.3.5.1 | sdhC | Escherichia coli | 12930948 | 44 |
| D | 1.3.5.1 | sdhD | Escherichia coli | 12930949 | 45 |
| E | 1.1.1.28 | idhA | Escherichia coli | 946315 | 46 |
| F | 2.8.3.1 | pct | Clostridium | 7242549 | 47 |
| | | | propionicum | | |
| F | 6.2.1.1 | acs1 | Saccharomyces | 851245 | 48 |
| | | | cerevisiae | | |
| G | 4.2.1.54 | lcdA | Clostridium | 343794933 | 49 |
| | | | propionicum | | |
| G | 4.2.1.54 | lcdA | Clostridium | 343794933 | 50 |
| | | | propionicum | | |
| G | 4.2.1.54 | lcdB | Clostridium | 343794931 | 51 |
| | | | propionicum | | |
| G | 4.2.1.54 | lcdC | Clostridium | 343794935 | 52 |
| | | | propionicum | | |
| H | 2.3.1 | paaJ | Escherichia coli | 12934018 | 53 |
| H | 2.3.1 | phaD | Pseudomonas putida | 10441755 | 54 |
| H | 2.3.1 | pcaF | Acinetobacter | 11639550 | 55 |
| | | | calcoaceticus | | |
| H | 2.3.1 | fadA | Aeromonas | 4490319 | 56 |
| | | | hydrophlai | | |
| H | 2.3.1 | atoB | Aeromonas | 4997503 | 57 |
| | | | salmonicida | | |
| H | 2.3.1 | pcaF | Pseudomonas | 4383639 | 58 |
| | | | aeroginosa | | |
| H | 2.3.1 | bktB | Ralstonia eutropha | 428815 | 59 |
| H | 2.3.1 | phaA | Cupriavidus necator | 10921806 | 60 |
| H | 2.3.1 | Clo1313_1716 | Clostridium | 12421448 | 61 |
| | | | thermocellum | | |
| I | 1.1.1.35 | fadB | Escherichia coli | 12934454 | 62 |
| I | 1.1.1.35 | yfcX | Escherichia coli | 12931539 | 63 |
| I | 1.1.1.36 | phbB | Cupriavidus necator | 10920675 | 64 |
| I | 1.1.1.36 | phaB | Rastonia | 9410631 | 65 |
| | | | solanacearum | | |
| J | 3.1.2 | Orf1 | Azoarcus evansii | 23664428 | 66 |
| J | 3.1.2 | COG0824 | Magnetospirillum | 46200680 | 67 |
| | | | magnetotacticum | | |
| J | 3.1.2 | Jann_0674 | Jannaschia sp. | 89052491 | 68 |
| | | | CCS1 | | |
| J | 3.1.2 | SSE37_24444 | Sagittula stellata | 126729407 | 69 |
| J | 3.1.2 | entH | Escherichia coli | 1786813 | 70 |
| J | 2.8.3 | atoA | Escherichia coli | 2492994 | 71 |
| J | 2.8.3 | atoD | Escherichia coli | 2492990 | 72 |
| J | 2.8.3 | actA | Corynebacterium | 62391407 | 73 |
| | | | glutamicum | | |
| J | 2.8.3 | Cg0592 | Corynebacterium | 62289399 | 74 |
| | | | glutamicum | | |
| J | 2.8.3 | ctfA | Clostridium | 15004866 | 75 |
| | | | acetobutylicum′ | | |
| J | 2.8.3 | ctfB | Clostridium | 15004867 | 76 |
| | | | acetobutylicum | | |
| J | 2.8.3 | Cbei_4543 | Clostridium | 150019354 | 77 |
| | | | beijerinchii | | |
| J | 2.8.3 | PcaJ | Acinetobacter sp. | 141766 | 78 |
| | | | ADP1 | | |
| K | 4.1 | oleTJE | Jeotgalicoccus sp; | HQ709266 | 79 |
| | | | ATCC8456 | | |
| K | 4.1. | padA1 | Aspergillus niger | 145235771 | 80 |
| K | 4.1. | sdrA | Aspergillus niger | 145235769 | 81 |
| K | 4.1.1.33 | mvd | Picrophilus torridus | 2845318 | 82 |
| K | 4.1.1.33 | mvd | Saccharomyces | 855779 | 83 |
| | | | cerevisiae | | |
| K | 4.1.1.33 | mvd | Schizosaccharomyces | 162312575 | 84 |
| | | | pombe | | |
| K | 4.1.1.33 | mvd | Halorhabdus | 257051090 | 85 |
| | | | utahensis | | |
| K | 4.1.1.33 | mvd | Haloterrigena | 8741675 | 86 |
| | | | turkmenica | | |
| K | 4.1.1.33 | mvd | Leuconostoc kimchii | 9132821 | 87 |
| K | 4.1 | dvd | Halobacterium | 1447408 | 88 |
| | | | salinarum | | |
| K | 4.1 | dfd | Aspergillus clavatu | 12170895 | 89 |
| K | 4.1.1.33 | mvaD | Streptococcus | 11027973 | 90 |
| | | | pseudopneumoniae | | |
| K | 4.1.1.33 | mvaD | Lactobacillus | 8433456 | 91 |
| | | | rhamnosus | | |
| K | 4.1.1.33 | mvaD | Borrelia afzelii | 12158799 | 92 |
| K | 4.1.1.1 | pdc2 | Saccharomyces | 851654 | 93 |
| | | | cerevisiae | | |
| K | 4.1.1.1 | pdc1 | Escherichia coli | 12759328 | 94 |

TABLE 7
Exemplary genes coding for enzymes in Table 3 and FIG. 3.

| | | | | Gene ID (GI) or | |
|---|---|---|---|---|---|
| Enzyme | | | | Accession Number | SEQ ID |
| No. | EC No. | Gene | Organism | (AN) | NO: |
| A | 4.1.1.49 | pckA | Escherichia coli | 12933187 | 95 |
| A | 4.1.1.49 | pckA | Actinobacillus | 5349670 | 96 |
| | | | succinogenes | | |
| B | 1.1.1.37 | mdh1 | Escherichia coli | 12931785 | 97 |
| B | 1.1.1.37 | mdh2 | Saccharomyces | 853994 | 98 |
| | | | cerevisiae | | |
| B | 1.1.5.4 | mqo | Escherichia coli | 12933223 | 99 |
| C | 4.2.1.2 | fum1 | Saccharomyces | 855866 | 100 |
| | | | cerevisiae | | |
| C | 4.2.1.2 | fumA | Escherichia coli | 12934128 | 101 |
| C | 4.2.1.2 | fumB | Escherichia coli | 12933156 | 102 |
| C | 4.2.1.2 | fumC | Escherichia coli | 12934129 | 103 |
| D | 1.3.99.1 | frdA | Escherichia coli | 12931889 | 104 |
| D | 1.3.99.1 | frdB | Escherichia coli | 12933707 | 105 |
| D | 1.3.99.1 | frdC | Escherichia coli | 12933706 | 106 |
| D | 1.3.99.1 | frdD | Escherichia coli | 12933705 | 107 |
| D | 1.3.5.1 | sdhA | Escherichia coli | 12933913 | 108 |
| D | 1.3.5.1 | sdhB | Escherichia coli | 12932956 | 109 |
| D | 1.3.5.1 | sdhC | Escherichia coli | 12930948 | 110 |
| D | 1.3.5.1 | sdhD | Escherichia coli | 12930949 | 111 |
| E | 1.1.1.28 | idhA | Escherichia coli | 946315 | 112 |
| F | 2.8.3.1 | pct | Clostridium | 7242549 | 113 |
| | | | propionicum | | |
| F | 6.2.1.1 | acs1 | Saccharomyces | 851245 | 114 |
| | | | cerevisiae | | |
| G | 4.2.1.54 | lcdA | Clostridium | 343794933 | 115 |
| | | | propionicum | | |
| G | 4.2.1.54 | lcdB | Clostridium | 343794931 | 116 |
| | | | propionicum | | |
| G | 4.2.1.54 | lcdC | Clostridium | 343794935 | 117 |
| | | | propionicum | | |
| H | 4.2.1.116 | Med_2001 | Metallosphaera | 5103388 | 118 |
| | | | sedula | | |
| H | 4.2.1.116 | BS732_3413 | Bacillus subtilis | M2W248 | 119 |
| H | 4.2.1.116 | CNE_2c24410 | Cupriavidus necator | 10922910 | 120 |
| I | 2.3.1 | paaJ | Escherichia coli | 12934018 | 121 |
| I | 2.3.1 | phaD | Pseudomonas putida | 10441755 | 122 |
| I | 2.3.1 | pcaF | Acinetobacter | 11639550 | 123 |
| | | | calcoaceticus | | |
| I | 2.3.1 | fadA | Aeromonas | 4490319 | 124 |
| | | | hydrophila | | |
| I | 2.3.1 | atoB | Aeromonas | 4997503 | 125 |
| | | | salmonicida | | |
| I | 2.3.1 | pcaF | Pseudomonas | 4383639 | 126 |
| | | | aeroginosa | | |
| I | 2.3.1 | bktB | Ralstonia eutropha | 428815 | 127 |
| I | 2.3.1 | phaA | Cupriavidus necator | 10921806 | 128 |
| I | 2.3.1 | Clo1313_1716 | Clostridium | 12421448 | 129 |
| | | | thermocellum | | |
| J | 1.1.1.35 | fadB | Escherichia coli | 12934454 | 130 |
| J | 1.1.1.35 | yfcX | Escherichia coli | 12931539 | 131 |
| J | 1.1.1.36 | phbB | Cupriavidus necator | 10920675 | 132 |
| J | 1.1.1.36 | phaB | Rastonia | 9410631 | 133 |
| | | | solanacearum | | |
| K | 4.2.1.80 | mhpD | Escherichia coli | 87081722 | 134 |
| K | 4.2.1.132 | ctmF | Pseudomonas putida | 1263188 | 135 |
| K | 4.2.1 | hpaH | Escherichia coli | 8178258 | 136 |
| K | 4.2.1.33/ | cnbE | Methanocaldococcus | 2122345 | 137 |
| | 4.2.1.35 | | jannaschii | | |
| K | 4.2.1.85 | dmdA | Eubacterium | 9884634 | 138 |
| | | | limosum | | |
| K | 4.2.1.85 | dmdB | Eubacterium | 9884633 | 139 |
| | | | limosum | | |
| K | 4.2.1.55 | crt | Clostridium | 1118895 | 140 |
| | | | acetobutylicum | | |
| K | 4.2.1.54 | lcdA | Clostridium | 343794933 | 141 |
| | | | propionicum | | |
| K | 4.2.1.54 | lcdB | Clostridium | 343794931 | 142 |
| | | | propionicum | | |
| K | 4.2.1.54 | lcdC | Clostridium | 343794935 | 143 |
| | | | propionicum | | |
| | 3.1.2 | Orf1 | Azoarcus evansii | 23664428 | 144 |
| L | 3.1.2 | COG0824 | Magnetospirillum | 46200680 | 145 |
| | | | magnetotacticum | | |
| L | 3.1.2 | Jann_0674 | Jannaschia sp. | 89052491 | 146 |
| | | | CCS1 | | |
| L | 3.1.2 | SSE37_24444 | Sagittula stellata | 126729407 | 147 |
| L | 3.1.2 | entH | Escherichia coli | 1786813 | 148 |
| L | 2.8.3 | atoA | Escherichia coli | 2492994 | 149 |
| L | 2.8.3 | atoD | Escherichia coli | 2492990 | 150 |
| L | 2.8.3 | actA | Corynebacterium | 62391407 | 151 |
| | | | glutamicum | | |
| L | 2.8.3 | Cg0592 | Corynebacterium | 62289399 | 152 |
| | | | glutamicum | | |
| L | 2.8.3 | ctfA | Clostridium | 15004866 | 153 |
| | | | acetobutylicum′ | | |
| L | 2.8.3 | ctfB | Clostridium | 15004867 | 154 |
| | | | acetobutylicum | | |
| L | 2.8.3 | Cbei_4543 | Clostridium | 150019354 | 155 |
| | | | beijerinchii | | |
| L | 2.8.3 | PcaJ | Acinetobacter sp. | 141776 | 156 |
| | | | ADP1 | | |
| M | 4.1. | oleTJE | Jeotgalicoccus sp; | HQ709266 | 157 |
| | | | ATCC8456 | | |
| M | 4.1. | padA1 | Aspergillus niger | 145235771 | 158 |
| M | 4.1. | sdrA | Aspergillus niger | 145235769 | 159 |
| M | 4.1.1.33 | mvd | Picrophilus torridus | 2845318 | 160 |
| M | 4.1.1.33 | mvd | Saccharomyces | 855779 | 161 |
| | | | cerevisiae | | |
| M | 4.1.1.33 | mvd | Schizosaccharomyces | 162312575 | 162 |
| | | | pombe | | |
| M | 4.1.1.33 | mvd | Halorhabdus | 257051090 | 163 |
| | | | utahensis | | |
| M | 4.1.1.33 | mvd | Haloterrigena | 8741675 | 164 |
| | | | turkmenica | | |
| M | 4.1.1.33 | mvd | Leuconostoc kimchii | 9132821 | 165 |
| M | 4.1 | dvd | Halobacterium | 1447408 | 166 |
| | | | salinarum | | |
| M | 4.1 | dfd | Aspergillus clavatu | 12170895 | 167 |
| M | 4.1.1.33 | mvaD | Streptococcus | 11027973 | 168 |
| | | | pseudopneumoniae | | |
| M | 4.1.1.33 | mvaD | Lactobacillus | 8433456 | 169 |
| | | | rhamnosus | | |
| M | 4.1.1.33 | mvaD | Borrelia afzelii | 12158799 | 170 |
| M | 4.1.1.1 | pdc2 | Saccharomyces | 851654 | 171 |
| | | | cerevisiae | | |
| M | 4.1.1.1 | pdc1 | Escherichia coli | 12759328 | 172 |

TABLE 8
Exemplary genes coding for enzymes in Table 4 and FIG. 4.

| | | | | Gene ID (GI) or | |
|---|---|---|---|---|---|
| Enzyme | | | | Accession Number | SEQ ID |
| No. | EC No. | Gene | Organism | (AN) | NO: |
| A | 4.1.1.49 | pckA | Escherichia coli | 12933187 | 173 |
| A | 4.1.1.49 | pckA | Actinobacillus | 5349670 | 174 |
| | | | succinogenes | | |
| B | 1.1.1.37 | mdh1 | Escherichia coli | 12931785 | 175 |
| B | 1.1.1.37 | mdh2 | Saccharomyces | 853994 | 176 |
| | | | cerevisiae | | |
| B | 1.1.5.4 | mqo | Escherichia coli | 12933223 | 177 |
| C | 4.2.1.2 | fum1 | Saccharomyces | 855866 | 178 |
| | | | cerevisiae | | |
| C | 4.2.1.2 | fumA | Escherichia coli | 12934128 | 179 |
| C | 4.2.1.2 | fumB | Escherichia coli | 12933156 | 180 |
| C | 4.2.1.2 | fumC | Escherichia coli | 12934129 | 181 |
| D | 1.3.99.1 | frdA | Escherichia coli | 12931889 | 182 |
| D | 1.3.99.1 | frdB | Escherichia coli | 12933707 | 183 |
| D | 1.3.99.1 | frdC | Escherichia coli | 12933706 | 184 |
| D | 1.3.99.1 | frdD | Escherichia coli | 12933705 | 185 |
| D | 1.3.5.1 | sdhA | Escherichia coli | 12933913 | 186 |
| D | 1.3.5.1 | sdhB | Escherichia coli | 12932956 | 187 |
| D | 1.3.5.1 | sdhC | Escherichia coli | 12930948 | 188 |
| D | 1.3.5.1 | sdhD | Escherichia coli | 12930949 | 189 |
| E | 2.3.1.9 | thlA | Clostridium | 3309200 | 190 |
| | | | acetobutylicum | | |
| E | 2.3.1.9 | Erg10 | Saccharomyces | 3309200 | 191 |
| | | | cerevisiae | | |
| F | 2.3.1.54/ | PFLA/B | Escherichia coli | | |
| | 1.97.1.4 | | | | |
| F | 2.3.1.54/ | PFLA/B | Neocallimastix | | |
| | 1.97.1.4 | | frontalis | | |
| G | 1.2.1.2 | fdhF | Escherichia coli | 12933956 | 192 |
| G | 1.2.2.1 | fdnH | Escherichia coli | 12933907 | 193 |
| G | 1.2.1.2 | fdh1 | Saccharomyces | 854570 | 194 |
| | | | cerevisiae | | |
| G | 1.2.1.2 | CLJU_c06990 | Clostridium | 9444316 | 195 |
| | | | ljungdahlii | | |
| G | 1.2.1.2 | CLJU_c07020 | Clostridium | 9444319 | 196 |
| | | | ljungdahlii | | |
| H | 2.8.3.16 | frc | Escherichia coli | 12931869 | 197 |
| H | 2.8.3.16 | frc | Shigella flexneri | 4209557 | 198 |
| H | 2.8.3.16 | frc | Streptomyces | 1213305 | 199 |
| | | | avermitilis | | |
| H | 2.8.3.16 | frc | Oligotropha | 10846643 | 200 |
| | | | carboxidovorans | | |
| H | 2.8.3.16 | frc | Rhodopseudomonas | 2688995 | 201 |
| | | | palustris | | |
| I | 2.3.1.174 | phaD | Pseudomonas putida | 10441755 | 202 |
| I | 2.3.1.174 | pcaF | Acinetobacter | 11639550 | 203 |
| | | | calcoaceticus | | |
| I | 2.3.1.174 | fadA | Aeromonas hydrophila | 4490319 | 204 |
| I | 2.3.1.174 | atoB | Aeromonas | 4997503 | 205 |
| | | | salmonicida | | |
| I | 2.3.1.174 | pcaF | Pseudomonas | 4383639 | 206 |
| | | | aeroginosa | | |
| I | 2.3.1.174 | bktB | Ralstonia eutropha | 428815 | 207 |
| I | 2.3.1.174 | phaA | Cupriavidus necator | 10921806 | 208 |
| I | 2.3.1.174 | Clo1313_1716 | Clostridium | 12421448 | 209 |
| | | | thermocellum | | |
| J, K, L | 1.1.1.35 | fadB | Escherichia coli | 12934454 | 210 |
| J, K, L | 1.1.1.35 | yfcX | Escherichia coli | 12931539 | 211 |
| J, K, L | 1.1.1.36 | phbB | Cupriavidus necator | 10920675 | 212 |
| J, K, L | 1.1.1.36 | phaB | Rastonia | 9410631 | 213 |
| | | | solanacearum | | |
| M | 4.2.1.80 | mhpD | Escherichia coli | 87081722 | 214 |
| M | 4.2.1.132 | ctmF | Pseudomonas putida | 1263188 | 215 |
| M | 4.2.1 | hpaH | Escherichia coli | 8178258 | 216 |
| M | 4.2.1.33/ | cnbE | Methanocaldococcus | 2122345 | 217 |
| | 4.2.1.35 | | jannaschii | | |
| M | 4.2.1.85 | dmdA | Eubacterium limosum | 9884634 | 218 |
| M | 4.2.1.85 | dmdB | Eubacterium limosum | 9884633 | 219 |
| M | 4.2.1.55 | crt | Clostridium | 1118895 | 220 |
| | | | acetobutylicum | | |
| M | 4.2.1.54 | lcdA | Clostridium | 343794933 | 221 |
| | | | propionicum | | |
| M | 4.2.1.54 | lcdB | Clostridium | 343794931 | 222 |
| | | | propionicum | | |
| M | 4.2.1.54 | lcdC | Clostridium | 343794935 | 223 |
| | | | propionicum | | |
| N | 3.1.2 | Orf1 | Azoarcus evansii | 23664428 | 224 |
| N | 3.1.2 | COG0824 | Magnetospirillum | 46200680 | 225 |
| | | | magnetotacticum | | |
| N | 3.1.2 | Jann_0674 | Jannaschia sp. CCS1 | 89052491 | 226 |
| N | 3.1.2 | SSE37_24444 | Sagittula stellata | 126729407 | 227 |
| N | 3.1.2 | entH | Escherichia coli | 1786813 | 228 |
| N | 2.8.3 | atoA | Escherichia coli | 2492994 | 229 |
| N | 2.8.3 | atoD | Escherichia coli | 2492990 | 230 |
| N | 2.8.3 | actA | Corynebacterium | 62391407 | 231 |
| | | | glutamicum | | |
| N | 2.8.3 | Cg0592 | Corynebacterium | 62289399 | 232 |
| | | | glutamicum | | |
| N | 2.8.3 | ctfA | Clostridium | 15004866 | 233 |
| | | | acetobutylicum′ | | |
| N | 2.8.3 | ctfB | Clostridium | 15004867 | 234 |
| | | | acetobutylicum | | |
| N | 2.8.3 | Cbei_4543 | Clostridium | 150019354 | 235 |
| | | | beijerinchii | | |
| N | 2.8.3 | PcaJ | Acinetobacter sp. | 141776 | 236 |
| | | | ADP1 | | |
| O | 4.1. | oleTJE | Jeotgalicoccus sp; | HQ709266 | 237 |
| | | | ATCC8456 | | |
| O | 4.1. | padA1 | Aspergillus niger | 145235771 | 238 |
| O | 4.1. | sdrA | Aspergillus niger | 145235769 | 239 |
| O | 4.1.1.33 | mvd | Picrophilus torridus | 2845318 | 240 |
| O | 4.1.1.33 | mvd | Saccharomyces | 855779 | 241 |
| | | | cerevisiae | | |
| O | 4.1.1.33 | mvd | Schizosaccharomyces | 162312575 | 242 |
| | | | pombe | | |
| O | 4.1.1.33 | mvd | Halorhabdus | 257051090 | 243 |
| | | | utahensis | | |
| O | 4.1.1.33 | mvd | Haloterrigena | 8741675 | 244 |
| | | | turkmenica | | |
| O | 4.1.1.33 | mvd | Leuconostoc kimchii | 9132821 | 245 |
| O | 4.1 | dvd | Halobacterium | 1447408 | 246 |
| | | | salinarum | | |
| O | 4.1 | dfd | Aspergillus clavatu | 12170895 | 247 |
| O | 4.1.1.33 | mvaD | Streptococcus | 11027973 | 248 |
| | | | pseudopneumoniae | | |
| O | 4.1.1.33 | mvaD | Lactobacillus | 8433456 | 249 |
| | | | rhamnosus | | |
| O | 4.1.1.33 | mvaD | Borrelia afzelii | 12158799 | 250 |
| O | 4.1.1.1 | pdc2 | Saccharomyces | 851654 | 251 |
| | | | cerevisiae | | |
| O | 4.1.1.1 | pdc1 | Escherichia coli | 12759328 | 252 |

Methods for the Co-Production of Butadiene with Succinate

Butadiene and succinate may be co-produced by contacting any of the disclosed genetically modified microorganisms with a fermentable carbon source. Such methods may preferably comprise contacting a fermentable carbon source with a microorganism comprising one or more polynucleotides coding for enzymes in a pathway that catalyze the conversion of the fermentable carbon source into any of the intermediates provided in either of Tables 1-4 or FIGS. 1-4 and one or more polynucleotides coding for enzymes in a pathway that catalyze a conversion one or more of the intermediates provided in FIGS. 1-4 (Tables 1-4) to butadiene and succinate in a fermentation media; and expressing the one or more polynucleotides coding for the enzymes in the pathway that catalyzes the conversion of the fermentable carbon source into one or more of the intermediates provided in FIGS. 1-4 (Tables 1-4) and one or more polynucleotides coding for enzymes in a pathway that catalyzes the conversion of one or more intermediates provided in FIGS. 1-4 (Tables 1-4) to butadiene and succinate.

The metabolic pathways that lead to the production of industrially important compounds involve oxidation-reduction (redox) reactions. For example, during fermentation, glucose is oxidized in a series of enzymatic reactions into smaller molecules with the concomitant release of energy. The electrons released are transferred from one reaction to another through universal electron carriers, such Nicotinamide Adenine Dinucleotide (NAD) and Nicotinamide Adenine Dinucleotide Phosphate (NAD(P)), which act as cofactors for oxidoreductase enzymes. In microbial catabolism, glucose is oxidized by enzymes using the oxidized form of the cofactors (NAD(P)+ and/or NAD+) as cofactor thus generating reducing equivalents in the form of the reduced cofactor (NAD(P)H and NADH). In order for fermentation to continue, redox-balanced metabolism is required, i.e., the cofactors must be regenerated by the reduction of microbial cell metabolic compounds.

Microorganism-catalyzed fermentation for the production of natural products is a widely known application of biocatalysis. Industrial microorganisms can affect multistep conversions of renewable feedstocks to high value chemical products in a single reactor. Products of microorganism-catalyzed fermentation processes range from chemicals such as ethanol, lactic acid, amino acids and vitamins, to high value small molecule pharmaceuticals, protein pharmaceuticals, and industrial enzymes. In many of these processes, the biocatalysts are whole-cell microorganisms, including microorganisms that have been genetically modified to express heterologous genes.

Some key parameters for efficient microorganism-catalyzed fermentation processes include the ability to grow microorganisms to a greater cell density, increased yield of desired products, increased amount of volumetric productivity, removal of unwanted co-metabolites, improved utilization of inexpensive carbon and nitrogen sources, adaptation to varying fermenter conditions, increased production of a primary metabolite, increased production of a secondary metabolite, increased tolerance to acidic conditions, increased tolerance to basic conditions, increased tolerance to organic solvents, increased tolerance to high salt conditions and increased tolerance to high or low temperatures. Inefficiencies in any of these parameters can result in high manufacturing costs, inability to capture or maintain market share, and/or failure to bring fermented end-products to market.

The methods and compositions of the present disclosure can be adapted to conventional fermentation bioreactors (e.g., batch, fed-batch, cell recycle, and continuous fermentation).

In some embodiments, a microorganism (e.g., a genetically modified microorganism) as provided herein is cultivated in liquid fermentation media (i.e., a submerged culture) which leads to excretion of the fermented product(s) into the fermentation media. In one embodiment, the fermented end product(s) can be isolated from the fermentation media using any suitable method known in the art.

In some embodiments, formation of the fermented product occurs during an initial, fast growth period of the microorganism. In one embodiment, formation of the fermented product occurs during a second period in which the culture is maintained in a slow-growing or quiescent state. In one embodiment, formation of the fermented product occurs during more than one growth period of the microorganism. In such embodiments, the amount of fermented product formed per unit of time is generally a function of the metabolic activity of the microorganism, the physiological culture conditions (e.g., pH, temperature, medium composition), and the amount of microorganisms present in the fermentation process.

In some embodiments, the fermentation product is recovered from the periplasm or culture medium as a secreted metabolite. In one embodiment, the fermentation product is extracted from the microorganism, for example when the microorganism lacks a secretory signal corresponding to the fermentation product. In one embodiment, the microorganisms are ruptured and the culture medium or lysate is centrifuged to remove particulate cell debris. The membrane and soluble protein fractions may then be separated if necessary. The fermentation product of interest may then be purified from the remaining supernatant solution or suspension by, for example, distillation, fractionation, chromatography, precipitation, filtration, and the like. In one embodiment, the microorganism cells (or portions thereof) may be used as biocatalysts or for other functions in a subsequent process without substantial purification.

Most industrial-scale butadiene and succinate, adipate, and/or butanol production processes are operated in the presence of measurable numbers of bacterial contaminants. Bacterial contamination causes a reduction in product yield and an inhibition of yeast growth. Few processes have been developed to control bacterial contaminations during fermentation. One of the most widely used processes is acid washing. Cells are collected from the fermentation broth, and sulfuric acid is used to adjust the pH of the cell paste to 2.0, which is kept for 2 h before being returned to the fermenter. This method can be successfully applied to a batch fermentation to increase productivity.

Methods for the Production of Polybutadiene and Other Compounds from Butadiene

Butadiene is gaseous at room temperature or in fermentative conditions (20-45° C.), and their production from a fermentation process results in a gas that could accumulate in the headspace of a fermentation tank, and be siphoned and concentrated. Butadiene may be purified from fermentation of gases, including gaseous alcohol, CO₂ and other compound by solvent extraction, cryogenic processes, distillation, fractionation, chromatography, precipitation, filtration, and the like.

Butadiene produced via any of the processes or methods disclosed herein may be converted to polybutadiene. Alternatively, butadiene produced via methods disclosed herein may be polymerized with other olefins to form copolymers such as acrylonitrile-butadiene-styrene (ABS), acrylonitrile-butadiene (ABR), or styrene-butadiene (SBR) copolymers, BR butyl rubber (RB), poly butadiene rubber (PBR), nitrile rubber and polychloroprene (Neoprene). Those synthetic rubbers or plastic elastomers applications include productions of tires, plastic materials, sole, shoe hills, technical goods, home appliance, neoprene, paper coatings, gloves, gaskets and seals.

Methods for the Production of Compounds from Succinate, Adipate, or Butanol

Succinate may be used themselves for a variety of commercial applications or may be used as an intermediate in the production of other compounds.

Succinate may be used as a chemical intermediate, in medicine, the manufacture of lacquers, and to make perfume esters. It is also used in foods as a sequestrant, buffer, and a neutralizing agent. Succinate may also be used in the production of pigments, solvents, detergents, metal plating and polymers such as polybutylene succinate, which can be used to replace conventional plastics in applications such as flexible packaging, agricultural films and compostable bags.

Without further description, it is believed that one of ordinary skill in the art may, using the preceding description and the following illustrative examples, make and utilize the agents of the present disclosure and practice the claimed methods. The following working examples are provided to facilitate the practice of the present disclosure, and are not to be construed as limiting in any way the remainder of the disclosure.

### EXAMPLES

### Example 1

### Modification of Microorganism for Production of Butadiene and Succinate

A microorganism such as a bacterium is genetically modified to produce butadiene and succinate from a fermentable carbon source including, for example, glucose.

In an exemplary method, a microorganism may be genetically engineered by any methods known in the art to comprise: i.) one or more polynucleotides coding for enzymes in a pathway that catalyze a conversion of the fermentable carbon source to a crotonyl-CoA, an acryloyl-CoA, a 3-hydroxypropionyl-CoA, and/or a formyl-CoA intermediate and one or more polynucleotides coding for enzymes in a pathway that catalyze a conversion of the crotonyl-CoA, the acryloyl-CoA, the 3-hydroxypropionyl-CoA, and/or the formyl-CoA intermediate to butadiene; and ii) one or more polynucleotides coding for enzymes in a pathway that catalyze a conversion of the fermentable carbon source to an oxalacetate intermediate and one or more polynucleotides coding for enzymes in a pathway that catalyze a conversion of the oxalacetate intermediate to succinate.

Alternatively, a microorganism that lacks one or more enzymes (e.g., one or more functional enzymes that are catalytically active) for the conversion of a fermentable carbon source to butadiene and succinate may be genetically modified to comprise one or more polynucleotides coding for enzymes (e.g., functional enzymes including, for example any enzyme disclosed herein) in a pathway that the microorganism lacks to catalyze a conversion of the fermentable carbon source to butadiene and succinate.

### Example 2

### Fermentation of Glucose by Genetically Modified Microorganism to Produce Butadiene and Succinate

A genetically modified microorganism, as produced in Example 1 above, may be used to ferment a carbon source producing butadiene and succinate.

In an exemplary method, a previously-sterilized culture medium comprising a fermentable carbon source (e.g., 9 g/L glucose, 1 g/L KH2PO4, 2 g/L (NH4)₂HPO4, 5 mg/L FeSO₄.7H₂O, 10 mg/L MgSO4.7H₂O, 2.5 mg/L MnSO4.H2O, 10 mg/L CaCl2.6H2O, 10 mg/L CoCl2.6H2O, and 10 g/L yeast extract) is charged in a bioreactor.

During fermentation, anaerobic conditions are maintained by, for example, sparging nitrogen through the culture medium. A suitable temperature for fermentation (e.g., about 30° C.) is maintained using any method known in the art. A near physiological pH (e.g., about 6.5) is maintained by, for example, automatic addition of sodium hydroxide. The bioreactor is agitated at, for example, about 50 rpm. Fermentation is allowed to run to completion.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein can be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary, only, with the true scope and spirit of the disclosure being indicated by the following claims. All references, patents, and patent applications referred to in this application are herein incorporated by reference in their entirety.

1. A method of co-producing butadiene and succinate from a fermentable carbon source, the method comprising:
a.) providing a fermentable carbon source;
b.) expressing one or more polynucleotides in a microorganism that encode one or more enzymes in a pathway that catalyze a conversion of the fermentable carbon source to one or more intermediates in a pathway for the production of butadiene and succinate,
c.) expressing one or more polynucleotides in the microorganism that encode one or more enzymes in a pathway that catalyze a conversion of the one or more intermediates into butadiene and succinate; and
d.) contacting the fermentable carbon source with the microorganism,
wherein the one or more intermediates in the pathway for the production of butadiene are selected from the group consisting of: crotonyl-CoA, acryloyl-CoA, 3-hydroxypropionyl-CoA, and formyl-CoA, and wherein the co-production method is anaerobic.

2. The method of claim 1, wherein the enzymes that catalyze the conversion of the fermentable carbon source to one or more intermediates in the pathway for the production of butadiene and succinate are set forth in any one of FIGS. 1-4.

3. The method of claim 1, wherein the enzymes that catalyze the conversion of the one or more intermediates to butadiene and succinate are set forth in any one of FIGS. 1-4.

4. The method of claim 1, wherein the microorganism is a bacteria selected from the genera consisting of: Propionibacterium, Propionispira, Clostridium, Bacillus, Escherichia, Pelobacter, and Lactobacillus.

5. The method of claim 1, wherein the microorganism is a eukaryote elected from the group consisting of yeast, filamentous fungi, protozoa, and algae.

6. The method of claim 5, wherein the yeast is Saccharomyces cerevisiae or Pichia pastoris.

7. The method of claim 1, wherein the fermentable carbon source comprises sugarcane juice, sugarcane molasses, hydrolyzed starch, hydrolyzed lignocellulosic materials, glucose, sucrose, fructose, lactate, lactose, xylose, pyruvate, or glycerol in any form or mixture thereof.

8. The method of claim 1, wherein the fermentable carbon source is a monosaccharide, oligosaccharide, or polysaccharide.

9. The method of claim 1, wherein the produced butadiene and succinate is secreted by the microorganism into the fermentation media.

10. The method of claim 9 further comprising recovering the produced butadiene, and succinate, adipate, and/or butanol from the fermentation media.

11. The method of claim 1, wherein the microorganism has been genetically modified to express the one or more polynucleotides coding for enzymes in a pathway that catalyze a conversion of the fermentable carbon source to one or more intermediates in a pathway for the production of butadiene and succinate, and one or more polynucleotides coding for enzymes in a pathway that catalyze a conversion of the one or more intermediates to butadiene and succinate.

12. A microorganism comprising one or more polynucleotides coding for enzymes in a pathway that catalyze a conversion of the fermentable carbon source to one or more intermediates in a pathway for the production of butadiene and succinate, and one or more polynucleotides coding for enzymes in a pathway that catalyze a conversion of the one or more intermediates to butadiene and succinate, wherein the one or more intermediates in the pathway for the production of butadiene are selected from the group consisting of: crotonyl-CoA, acryloyl-CoA, 3-hydroxypropionyl-CoA., and formyl-CoA.

13. The microorganism of claim 12, wherein the enzymes that catalyze a conversion of the fermentable carbon source to one or more intermediates in the pathway for the production of butadiene and succinate are set forth in any one of FIGS. 1-4.

14. The microorganism of claim 12, wherein the enzymes that catalyze a conversion of the one or more intermediates to butadiene and succinate are set forth in any one of FIGS. 1-4.

15. The microorganism of claim 12, wherein the microorganism is a bacteria selected from the genera consisting of: Propionibacterium, Propionispira, Clostridium, Bacillus, Escherichia, Pelobacter, and Lactobacillus.

16. The microorganism of claim 15, wherein the microorganism is a eukaryote selected from the group consisting of yeast, filamentous fungi, protozoa, and algae.

17. The microorganism of claim 16, wherein the yeast is Saccharomyces cerevisiae or Pichia pastoris.

18. The microorganism of claim 12, wherein the microorganism has been genetically modified to express the one or more polynucleotides coding for enzymes in a pathway that catalyze a conversion of the fermentable carbon source to one or more intermediates in a pathway for the production of butadiene and succinate, and one or more polynucleotides coding for enzymes in a pathway that catalyze a conversion of the one or more intermediates to butadiene and succinate.
